SEQUENCE LISTING (5 sequences)

SEQ ID NO: 1 - protein, 238 aa, Artificial Sequence
  Recombinant Protein
MSKGEELFTGVVPILVELDGDVNGHKFSVSGEGEGDATYGKLTLKFICTTGKLPVPWPTLVTTLTYGVQCFSRYPDHMKRHDFFKSAMPEGYVQERTIFFKDDGNYKTRAEVKFEGDTLVNRIELKGIDFKEDGNILGHKLEYNYNDHQVYIMADKQKNGIKANFKIRHNIEDGGVQLADHYQQNTPIGDGPVLLPDNHYLFTTSTLSKDPNEKRDHMVLLEFVTAAGITHGMDELYK

SEQ ID NO: 2 - DNA, 1665 bp, Artificial Sequence
  Synthetic polynucleotide
  synaptophysin (rat) (1)..(555)
  Super ecliptic pHluorin (556)..(1269)
  synaptophysin (rat) (1270)..(1665)
atggacgtggtgaatcagctggtggctgggggtcagttccgggtggtcaaggagccccttggcttcgtgaaggtgctgcagtgggtctttgccatcttcgcctttgctacgtgtggcagctacaccggggagcttcggctgagcgtggagtgtgccaacaagacggagagtgccctcaacatcgaagttgaattcgagtaccccttcaggctgcaccaagtgtactttgatgcaccctcctgcgtcaaagggggcactaccaagatcttcctggttggggactactcctcgtcggctgaattctttgtcaccgtggctgtgtttgccttcctctactccatgggggccctggccacctacatcttcctgcagaacaagtaccgagagaacaacaaagggcctatgatggactttctggctacagccgtgttcgctttcatgtggctagttagttcatcagcctgggccaaaggcctgtccgatgtgaagatggccacggacccagagaacattatcaaggagatgcccatgtgccgccagacagggaacaccggtatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttaacttatggtgttcaatgcttttcaagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataacgatcaccaggtgtacatcatggcagacaaacaaaagaatggaatcaaagctaacttcaaaattagacacaacattgaagatggaggcgttcaactagcagaccattatcaacaaaatactccaattggcgatgggcccgtccttttaccagacaaccattacctgtttacaacttctactctttcgaaagatcccaacgaaaagagagaccacatggtccttcttgagtttgtaacagctgctgggattacacatggcatggatgaactatacaaaaccggtcgccagacagggaacacatgcaaggaactgagggaccctgtgacttcaggactcaacacctcagtggtgtttggcttcctgaacctggtgctctgggttggcaacttatggttcgtgttcaaggagacaggctgggcagccccattcatgcgcgcacctccaggcgccccggaaaagcaaccagcacctggcgatgcctacggcgatgcgggctacgggcagggccccggaggctatgggccccaagactcctacgggcctcagggtggttatcaacccgattacgggcagccagccagcggtggcggtggctacgggcctcagggcgactatgggcagcaaggctatggccaacagggtgcgcccacctccttctccaatcagatgtaa

SEQ ID NO: 3 - DNA, 2472 bp, Artificial Sequence
  Synthetic polynucleotide
atggagttcagacaggaggagttccgaaaacttgcaggccgagcactgggtcgactgcatcgcttgctggagaagcggcaggagggagcagaaacactggagctcagcgctgacggccggcccgtcacaacccacactagagacccacctgtggtagactgtacctgctttggcctgcctagaaggtatatcattgcaatcatgtctgggcttggcttctgcatcagttttggaattagatgtaacctgggggttgcgattgtgagtatggtcaataactcaacgacacacagaggaagcacctccggtggatccggggggactgggggaagcatgtccaaaggcgaagaactgtttacaggcgtggtgccaatactcgtggaactcgacggcgatgttaacggacataagttttccgtgtccggcgagggtgagggagatgctacatatggaaaactgactctgaaatttatttgtaccaccgggaaactgcccgtgccatggcctaccctcgtcactacactgacgtatggggttcaatgtttcagtaggtatccggaccacatgaaaagacacgatttcttcaaaagcgcaatgcccgaaggctacgtacaagaacgcactatattcttcaaggacgacggcaactataaaacccgcgcagaggttaaatttgagggggacacactggtaaatcggatcgagctgaagggtatcgactttaaagaagacggaaacatattgggccataagctggaatacaactataatgatcatcaggtgtatatcatggccgataagcagaaaaatggaatcaaggcaaacttcaaaattaggcacaatattgaggatgggggcgtccagctggccgaccattaccagcagaatactccaataggcgatggaccggtgttgcttcctgataatcactacttgtttaccactagcaccctcagtaaggatcccaatgagaagcgggaccacatggtcctgctggagttcgtgactgccgctggcataactcatgggatggatgagctctacaaaggagggacagggggtaccggtggtagtggtggaaccggcggccatgtagtggtgcagaaggcccagttcaattgggacccagagacagtgggcttgatccacggctctttcttttggggttatatcgttactcaaatcccaggcggtttcatttgtcagaagttcgccgccaacagagtgttcggcttcgcaatagtagccacctccacactgaacatgctgattccgtctgcagcccgagtgcactatggctgtgtgatattcgtgagaatcctgcaaggtcttgtcgagggcgtgacctacccagcctgtcacgggatctggtccaaatgggctccaccactggagcgatccagactggcgaccactgcattctgtggcagttacgccggcgctgtcgtggctatgcccctggctggtgtgctggtgcagtacagcggatggagcagcgtgttctacgtttatggctctttcgggatcttttggtacctcttctggctgctcgtcagctacgaatcccccgctctgcacccttccatcagcgaggaggagcggaaatacattgaagacgcaatcggtgaatcagcaaagctgatgaaccccgtgacgaaattcaatacaccgtggagacggttttttacctccatgcccgtgtacgcgataattgttgcaaatttctgccggagctggacattctatctcctcctgattagccaacctgcctacttcgaagaggtcttcgggttcgagatctctaaagtgggtcttgtgtctgcgctccctcacctcgttatgaccattattgttcctatcggcgggcagatcgctgattttcttcggtcaagacatattatgtccactactaatgtgaggaagctgatgaattgcggtgggtttggcatggaggcgaccctgcttctggtggtgggttactctcattccaagggagtggcgatcagtttcctggtcctggccgtgggatttagcggctttgccataagcggttttaacgttaatcaccttgatattgctccccgctacgcatccattctcatggggatcagcaacggagtcgggactctgagtggaatggtttgcccaataatagtcggagcgatgactaaacataagacccgagaagaatggcagtacgtgttcctgattgctagtctggtccattacggaggcgtcatcttctatggcgtcttcgcctctggggaaaaacaaccatgggcagagcctgaagaaatgtccgaggaaaaatgtgggttcgtcgggcacgatcagcttgccggttctgatgagtccgagatggaggacgaggttgagccccctggggcgcctcctgcaccgcccccaagctacggcgcaacccatagcactgtgcagcctcctcggccacctcctccagtgcgggattactaa

SEQ ID NO: 4 - DNA, 619 bp, Artificial Sequence
  Synthetic polynucleotide
actaatctccccgcgggcactgcgtgtgacctcacccccctctgtgagggggttatttctctactttcgtgtctctgagtgtgcttccagtgcccccctccccccaaaaaatgccttctgagttgaatatcaacactacaaaccgagtatctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 5 - DNA, 2326 bp, Artificial Sequence
  Synthetic polynucleotide
  human synapsin I promoter (1)..(619)
  UTR (620)..(661)
  synaptophysin (rat) (662)..(1216)
  Super ecliptic pHluorin (1217)..(1930)
  synaptophysin (rat) (1931)..(2326)
actaatctccccgcgggcactgcgtgtgacctcacccccctctgtgagggggttatttctctactttcgtgtctctgagtgtgcttccagtgcccccctccccccaaaaaatgccttctgagttgaatatcaacactacaaaccgagtatctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaattcaagctgctagcaaggatccaccggtcgccaccatggacgtggtgaatcagctggtggctgggggtcagttccgggtggtcaaggagccccttggcttcgtgaaggtgctgcagtgggtctttgccatcttcgcctttgctacgtgtggcagctacaccggggagcttcggctgagcgtggagtgtgccaacaagacggagagtgccctcaacatcgaagttgaattcgagtaccccttcaggctgcaccaagtgtactttgatgcaccctcctgcgtcaaagggggcactaccaagatcttcctggttggggactactcctcgtcggctgaattctttgtcaccgtggctgtgtttgccttcctctactccatgggggccctggccacctacatcttcctgcagaacaagtaccgagagaacaacaaagggcctatgatggactttctggctacagccgtgttcgctttcatgtggctagttagttcatcagcctgggccaaaggcctgtccgatgtgaagatggccacggacccagagaacattatcaaggagatgcccatgtgccgccagacagggaacaccggtatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttaacttatggtgttcaatgcttttcaagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataacgatcaccaggtgtacatcatggcagacaaacaaaagaatggaatcaaagctaacttcaaaattagacacaacattgaagatggaggcgttcaactagcagaccattatcaacaaaatactccaattggcgatgggcccgtccttttaccagacaaccattacctgtttacaacttctactctttcgaaagatcccaacgaaaagagagaccacatggtccttcttgagtttgtaacagctgctgggattacacatggcatggatgaactatacaaaaccggtcgccagacagggaacacatgcaaggaactgagggaccctgtgacttcaggactcaacacctcagtggtgtttggcttcctgaacctggtgctctgggttggcaacttatggttcgtgttcaaggagacaggctgggcagccccattcatgcgcgcacctccaggcgccccggaaaagcaaccagcacctggcgatgcctacggcgatgcgggctacgggcagggccccggaggctatgggccccaagactcctacgggcctcagggtggttatcaacccgattacgggcagccagccagcggtggcggtggctacgggcctcagggcgactatgggcagcaaggctatggccaacagggtgcgcccacctccttctccaatcagatgtaa

(19) United States
(12) Patent Application Publication
(10) Pub. No.: US 2018/0136198 A1
(43) Pub. Date: May 17, 2018

(54) SYNAPTIC VESICLE CYCLING ASSAYS AND SYSTEMS

(71) Applicant: Q-State Biosciences, Inc., Cambridge, MA (US)

(72) Inventors: David J. Gerber, Somerville, MA (US); Jeffrey R. Cottrell, Sterling, MA (US); Timothy A. Ryan, New York, NY (US); Jonathan M. Levenson, Walpole, MA (US)

(73) Assignee: Q-State Biosciences, Inc, Cambridge, MA (US)

(21) Appl. No.: 15/868,200

(22) Filed: Jan. 11, 2018

Related U.S. Application Data
(63) Continuation of application No. 13/062,459, filed on Nov. 9, 2011.
(60) Provisional application No. 61/094,361, filed on Sep. 4, 2008.

(51) Int. Cl. G01N 33/50; G01N 33/542; G01N 33/68

(52) U.S. Cl. CPC G01N 33/5058; G01N 2500/10; G01N 33/6872; G01N 33/542

(57) ABSTRACT

The present invention provides, in part, platforms for analyzing an aspect of synaptic vesicle cycling. According to other aspects, the invention provides neuronal cell culture platform and platforms for analyzing an aspect of synaptic vesicle cycling. According to other aspects, the invention provides methods of measuring an aspect of synaptic vesicle cycling in a plurality of cells. According to other aspects, the invention provide methods for identifying a test agent as a modulator of an aspect of synaptic vesicle cycling.

### RELATED APPLICATIONS

This application is a continuation application which claims the benefit under 35 U.S.C § 120 of U.S. application Ser. No. 13/062,459, filed Nov. 9, 2011, which is a National Stage filing under 35 U.S.C. § 371 of International Application PCT/US2009/004932, filed Aug. 31, 2009, which was published under PCT Article 21(2) in English and claims the benefit under 35 U.S.C. § 119(e) to U.S. Provisional Application Ser. No. 61/094,361, filed on Sep. 4, 2008 and entitled “SYNAPTIC VESICLE CYCLING ASSAYS AND SYSTEMS,” each of which is herein incorporated by reference in its entirety.

### FIELD OF THE INVENTION

The invention relates to systems and methods for identifying modulators of synaptic transmission.

### BACKGROUND OF THE INVENTION

Synaptic vesicle cycling is central to neurotransmission. This process typically takes place at the nerve terminals and involves steps of vesicle exocytosis with neurotransmitter release, endocytosis of empty vesicles, and recycling and reuse of vesicles. Neurotransmitter transporters contained in synaptic vesicles load the vesicles with neurotransmitters. Loaded vesicles translocate to the terminal plasma membrane where they selectively dock close to the active zone and become fusion competent. Typically, a rise in intracellular calcium concentration triggers vesicle fusion and release of neurotransmitter into the synaptic cleft. Neurotransmitter can then bind and activate receptors. Following fusion, vesicle proteins and membrane are retrieved by clathrin-mediated endocytosis, refilled with neurotransmitter, and recycled for subsequent release. Thus, neurotransmission involves the proper cycling of synaptic vesicles through repetitive episodes of exocytosis and endocytosis. Disruptions of synaptic vesicle cycling may lead to various psychiatric and neurological disorders.

### SUMMARY OF THE INVENTION

The present invention is based, in part, on the development of novel platforms and methods that enable stimulation of action potentials in neuronal cells and detection of aspects of synaptic transmission in multiple neuronal cell cultures in parallel. The present invention, in some aspects, overcomes longstanding challenges associated with analyzing synaptic transmission in a high-throughput format. Thus, the present invention provides, in some aspects, platforms and methods for identifying modulators of synaptic transmission in a high-throughput manner. The present invention is also based on the discovery that using the novel platforms and methods disclosed herein, aspects of synaptic vesicle cycling can be detected in a high-throughput manner with high sensitivity, in some cases, using arrays of low numerical aperture air objective lenses. Accordingly, in some aspects, the present invention provides platforms and methods for analyzing aspects of synaptic vesicle cycling in a high-throughput manner. In other aspects, the invention provides neuronal cell culture platforms for analyzing aspects of synaptic vesicle cycling in multiple parallel cultures.

The present invention provides platforms, systems and methods for identifying modulators of synaptic vesicle cycling. Among other things, inventive methods and systems can identify reagents and tools for use in characterizing synaptic vesicle cycling pathways, as well as diagnostic and/or therapeutic agents for use in monitoring and/or modulating such pathways.

In some embodiments, the present invention provides synaptic vesicle cycling assays based on, for example, at least one aspect of synaptic vesicle cycling activity. In some embodiments, synaptic vesicle cycling assays monitor kinetics, efficiency, and/or other characteristics of the synaptic vesicle cycle (e.g., frequency, duration, and/or synaptic fatigue).

In some embodiments, the present invention provides systems and methods for identifying agents that modulate (e.g., stimulate and/or inhibit) synaptic vesicle cycling through the use of synaptic vesicle cycling assays in accordance with the invention.

Modulatory agents in accordance with the present invention may be any class of chemical entity (e.g., polypeptides, nucleic acids, antibodies, small molecules, carbohydrates, lipids, microorganisms, etc.)

Synaptic vesicle cycling assays utilized in accordance with the present invention typically comprise: a plurality of cells (e.g., comprising at least one cell having presynaptic terminals which exhibit functional synaptic vesicle cycling or at least one activity of synaptic vesicle cycling), a reporter (e.g., a reporter useful for tracking synaptic vesicle cycling or at least one activity of synaptic vesicle cycling), a stimulation system (e.g., electrical, acoustic, ultrasonic, or optical stimulation systems to trigger synaptic vesicle cycling, for example, via action potential initiation), and a detection system (e.g., an imaging system to capture signal generated by the reporter).

In some embodiments, synaptic vesicle cycling assays are arranged and/or performed in high-throughput format. For example, inventive methods in accordance with the present invention may include a step of measuring multiple synaptic vesicle cycling assays (e.g., at least 4, 16, or more synaptic vesicle cycling assays) simultaneously using a single instrument. In some embodiments, inventive methods in accordance with the present invention may include measuring at least 24 (e.g., at least 48, 96, 384 or more) synaptic vesicle cycling assays per hour on a single instrument. In some embodiments, instruments utilized in accordance with the present invention are adapted to screening multi-well plates (e.g., 24, 48, 96 or 384-well plates).

According to one aspect of the invention, a platform for analyzing an aspect of synaptic vesicle cycling is provided. The platform includes a) a plurality of wells; b) a plurality of electrode pairs, wherein each electrode pair is configured (i) for placement in a well, and (ii) to produce an electrical field suitable to induce synaptic vesicle cycling in a plurality of neuronal cells in the well; and c) a detection system comprising a plurality of detectors, wherein each detector is configured to detect a luminescent signal from a reporter molecule attached to a presynaptic protein of a neuronal cell present in a well, and wherein the presence of the luminescent signal is indicative of an aspect of synaptic vesicle cycling in the neuronal cell. In some embodiments, a plurality of the wells comprise a plurality of neuronal cells. In some embodiments, the plurality of neuronal cells in a well is in a range of 10 to 1,000,000 neuronal cells. In certain embodiments, the plurality of neuronal cells in a well is in a range of 1000 to 4000 cells/mm² of well bottom area. In some embodiments, the plurality of neuronal cells comprise at least two different neuronal cell types. In certain embodiments, the neuronal cells are primary neurons, optionally wherein the primary neurons are rat primary neurons. In certain embodiments, the neuronal cells are selected from the group consisting of: glutamatergic, GABAergic, dopaminergic, adrenergic, serotonergic, and cholinergic neuronal cells. In some embodiments, each electrode of an electrode pair has a substantially curvilinear surface. In some embodiments, the electrodes of each electrode pair are substantially concentric cylinders, and wherein the concentric cylinders are separated by an annular insulating material. In some embodiments, the platform also includes an electrode transfer system configured to operably position each electrode pair of the plurality of electrode pairs into one well of the plurality of wells. In certain embodiments, the platform also includes comprising a power source operably linked to the plurality of electrodes. In some embodiments, the power source is configured to apply a predetermined voltage across each electrode pair. In some embodiments, the voltage is in range of 1 V to 400 V. In certain embodiments, the voltage is in a range of 5 V to 20 V. In some embodiments, the platform also includes a pulse generator operably linked to the power supply and the plurality of electrode pairs, wherein the pulse generator is configured to apply a predetermined voltage pulse across each electrode pair. In some embodiments, the pulse generator is configured to apply a plurality of predetermined voltage pulses at a predetermined frequency for a predetermined time. In certain embodiments, the predetermined frequency is in a range of 0.2 Hz to 100 Hz. In some embodiments, the predetermined frequency is in a range of 10 Hz to 50 Hz. In some embodiments, predetermined time is up to 2 minutes. In some embodiments, the predetermined time is in a range of 0.1 to 20 seconds. In certain embodiments, the predetermined time is in a range of 5 to 15 seconds. In some embodiments, the duration of each pulse is in a range of up to 10 msec. In some embodiments, the duration of each pulse is in a range of 0.1 msec to 2 msec. In some embodiments, the duration between the initiation of each pulse is in a range of 0.1 to 5 msec. In certain embodiments, the number of pulses is in a range of 1 to 1000. In some embodiments, the platform also includes a computer operably linked to the pulse generator, wherein the computer is configured to control the voltage pulse. In some embodiments, each detector comprises an optical sensor. In some embodiments, each detector comprises an objective lens configured to collect a luminescent signal from a well. In certain embodiments, the objective lens is configured to collect a luminescent signal from a field area in a range of 0.2 mm to 5 mm. In some embodiments, the objective lens has a numerical aperture in a range 0.4 to 1.4. In some embodiments, the objective lens has a numerical aperture of 0.5. In certain embodiments, the objective lens is not an oil or water immersion lens. In some embodiments, the detection system comprises a charge-coupled device camera operably linked to each detector. In some embodiments, the plurality of detectors are configured to simultaneously detect signals from a plurality of wells. In certain embodiments, the detection system comprises a computer operably linked to the detectors, and wherein the computer is configured to transform luminescent signal from the detectors into data characterizing an aspect of synaptic vesicle cycling in a neuronal cell. In some embodiments, each detector is configured to detect a luminescent signal from a plurality of reporter molecules. In some embodiments, each detector is configured to detect a luminescent signal from a plurality of synapses. In certain embodiments, each detector is configured to detect a luminescent signal from a plurality of neuronal cells. In some embodiments, the synaptic vesicle protein is VAMP2, vGlut1, synaptophysin, vesicular GABA transporter; acetylcholine transporter, catecholamine transporter or synaptotagmin. In some embodiments, a plurality of the neuronal cells express a synaptic vesicle protein having a lumenal portion, wherein the synaptic vesicle protein is attached to a reporter molecule. In some embodiments, the reporter molecule is attached to the lumenal portion of the synaptic vesicle protein. In certain embodiments, the reporter molecule is a pH sensitive fluorescent protein. In some embodiments, the reporter molecule is a pHluorin. In some embodiments, the reporter molecule comprises a sequence set forth in SEQ ID NO: 1 (hSyn-SypHy). In some embodiments, at a pH in a range of 7.0 to 8.0, the pH sensitive reporter fluoresces with an intensity that is significantly greater than at a pH in a range of 5.0 to 6.0. In certain embodiments, at a pH in a range of 5.0 to 6.0, the pH sensitive reporter fluoresces with an intensity that is significantly greater than at a pH in a range of 7.0 to 8.0. In some embodiments, the luminescent signal is a fluorescent signal in a range of 475 nm to 525 nm.

According to another aspect of the invention, a platform for analyzing an aspect of synaptic vesicle cycling is provided. The platform including a) a plurality of wells, wherein each well comprises a plurality of neuronal cells; b) a plurality of electrode pairs, wherein each electrode pair is positioned within one of the plurality of wells, and wherein each electrode pair is configured to produce an electric field that is sufficient to induce synaptic vesicle cycling in a neuronal cell present in the well; and c) a detection system comprising a plurality of detectors, wherein each detector is configured to detect a luminescent signal from at least a subset of the plurality of neuronal cells, wherein the luminescent signal is indicative of an aspect of synaptic vesicle cycling. In some embodiments, the plurality of neuronal cells comprise a reporter molecule attached to a synaptic vesicle protein.

According to another aspect of the invention, a platform for analyzing an aspect of synaptic vesicle cycling is provided, the platform including a) a plurality of wells, wherein each well comprises a plurality of neuronal cells, and wherein a plurality of the neuronal cells comprise a reporter molecule attached to a synaptic vesicle protein; b) a stimulator system configured to induce synaptic vesicle cycling in neuronal cells present in the wells; and c) a detection system comprising a plurality of detectors, wherein each detector is configured to detect a luminescent signal from at least a subset of the plurality of neuronal cells present in a well, and wherein the luminescent signal is indicative of an aspect of synaptic vesicle cycling.

In certain embodiments, the synaptic vesicle protein comprises a lumenal portion. In some embodiments of any aforementioned aspects, the stimulator system comprises a plurality of electrode pairs, wherein each electrode pair is positioned within one of the plurality of wells, and wherein each electrode pair is configured to produce an electric field that is sufficient to induce synaptic vesicle cycling in a neuronal cell present in the well. In some embodiments of any aforementioned aspects, the plurality of neuronal cells in a well is in a range of 10 to 100,000 neuronal cells. In certain embodiments of any aforementioned platform, the plurality of neuronal cells in a well is in a range of 1000 to 4000 cells/mm² of well bottom area. In some embodiments of any aforementioned platform, the plurality of neuronal cells comprise at least two different neuronal cell types. In some embodiments of any aforementioned platform, the neuronal cells are primary neurons, optionally wherein the primary neurons are rat primary neurons. In some embodiments of any aforementioned platform, the neuronal cells are selected from the group consisting of: glutamatergic, GABAergic, dopaminergic, adrenergic, serotonergic, and cholinergic neuronal cells. In certain embodiments of any aforementioned platform, the neuronal cells comprise a transgene that expresses a synaptic vesicle protein, having a lumenal portion, fused to a reporter molecule. In some embodiments of any aforementioned platform, each electrode of an electrode pair has a substantially curvilinear surface. In some embodiments of any aforementioned platform, the electrodes of each electrode pair are substantially concentric cylinders, and wherein the concentric cylinders are separated by an annular insulating material. In some embodiments of any aforementioned platform, the platform also includes an electrode transfer system configured to operably position each electrode pair of the plurality of electrode pairs into one well of the plurality of wells. In certain embodiments of any aforementioned platform, the platform also includes a power source operably linked to the plurality of electrodes. In some embodiments of any aforementioned platform, the power source is configured to apply a predetermined voltage across each electrode pair. In some embodiments of any aforementioned platform, the voltage is in range of 1 V to 400 V. In certain embodiments of any aforementioned platform, the voltage is in a range of 5 V to 20 V. In some embodiments of any aforementioned platform, the platform also includes a pulse generator operably linked to the power source and the plurality of electrode pairs, wherein the pulse generator is configured to apply a predetermined voltage pulse across each electrode pair. In some embodiments of any aforementioned platform, the pulse generator is configured to apply a plurality of predetermined voltage pulses at a predetermined frequency for a predetermined time. In certain embodiments of any aforementioned platform, the predetermined frequency is in a range of 0.2 Hz to 100 Hz. In some embodiments of any aforementioned platform, the predetermined frequency is in a range of 10 Hz to 50 Hz. In some embodiments of any aforementioned platform, the predetermined time is up to 2 minutes. In some embodiments of any aforementioned platform, the predetermined time is in a range of 0.1 to 20 seconds. In certain embodiments of any aforementioned platform, the predetermined time is in a range of 5 to 15 seconds. In some embodiments of any aforementioned platform, the duration of each pulse is in a range of up to 10 msec. In some embodiments of any aforementioned platform, the duration of each pulse is in a range of 0.1 msec to 2 msec. In certain embodiments of any aforementioned platform, the duration between the initiation of each pulse is in a range of 0.1 to 5 msec. In some embodiments of any aforementioned platform, the number of pulses is in a range of 1 to 1000. In some embodiments of any aforementioned platform, the platform also includes a computer operably linked to the pulse generator, wherein the computer is configured to control the voltage pulse. In certain embodiments of any aforementioned platform, each detector comprises an optical sensor. In some embodiments of any aforementioned platform, each detector comprises an objective lens configured to collect a luminescent signal from a well. In some embodiments of any aforementioned platform, the objective lens is configured to collect a luminescent signal from a field area in a range of 0.2 mm to 5 mm. In some embodiments of any aforementioned platform, the objective lens has a numerical aperture in a range 0.4 to 1.4. In certain embodiments of any aforementioned platform, the objective lens has a numerical aperture of 0.5. In some embodiments of any aforementioned platform, the objective lens is not an oil or water immersion lens. In some embodiments of any aforementioned platform, the detection system comprises a charge-coupled device camera operably linked to each detector. In certain embodiments of any aforementioned platform, the plurality of detectors are configured to simultaneously detect signals from a plurality of wells. In some embodiments of any aforementioned platform, the detection system comprises a computer operably linked to the detectors, and wherein the computer is configured to transform luminescent signal from the detectors into data characterizing an aspect of synaptic vesicle cycling in a neuronal cell. In some embodiments of any aforementioned platform, each detector is configured to detect a luminescent signal from a plurality of reporter molecules. In some embodiments of any aforementioned platform, each detector is configured to detect a luminescent signal from a plurality of synapses. In certain embodiments of any aforementioned platform, each detector is configured to detect a luminescent signal from a plurality of neuronal cells. In some embodiments of any aforementioned platform, the synaptic vesicle protein is VAMP2, vGlut1, synaptophysin, vesicular GABA transporter; acetylcholine transporter, catecholamine transporter or synaptotagmin. In some embodiments of any aforementioned platform, the reporter molecule is attached to the lumenal portion of the synaptic vesicle protein. In certain embodiments of any aforementioned platform, the reporter molecule is a pH sensitive fluorescent protein. In some embodiments of any aforementioned platform, the reporter molecule is a pHluorin. In some embodiments of any aforementioned platform, the reporter molecule comprises a sequence set forth in SEQ ID NO: 1 (hSyn-SypHy). In certain embodiments of any aforementioned platform, at a pH in a range of 7.0 to 8.0, the pH sensitive reporter fluoresces with an intensity that is significantly greater than at a pH in a range of 5.0 to 6.0. In some embodiments of any aforementioned platform, at a pH in a range of 5.0 to 6.0, the pH sensitive reporter fluoresces with an intensity that is significantly greater than at a pH in a range of 7.0 to 8.0. In some embodiments of any aforementioned platform, the luminescent signal is a fluorescent signal in a range of 475 nm to 525 nm.

In yet another aspect of the invention, a platform for analyzing an aspect of synaptic vesicle cycling is provided. The platform including a) a plurality of wells; b) a plurality of electrode pairs, wherein each electrode pair is configured (i) for placement in a well, and (ii) to produce an electrical field suitable to induce synaptic vesicle cycling in a plurality of neuronal cells in the well; and c) a detection system comprising an objective lens configured to collect luminescent signal from a reporter molecule attached to a synaptic vesicle protein of a neuronal cell present in a well, and wherein the presence of the luminescent signal is indicative of an aspect of synaptic vesicle cycling in the neuronal cell. In some embodiments, a plurality of the wells comprise a plurality of neuronal cells.

According to another aspect of the invention, a neuronal cell culture platform for analyzing an aspect of synaptic vesicle cycling is provided. The platform includes a) a plurality of wells, wherein each well comprises a plurality of neuronal cells; b) a plurality of electrode pairs, wherein each electrode pair is positioned within one of the plurality of wells, and wherein each electrode pair is configured to produce an electric field that is sufficient to induce synaptic vesicle cycling in a neuronal cell present in the well; and c) a detection system comprising an objective lens configured to collect luminescent signal from a reporter molecule attached to a synaptic vesicle protein of a neuronal cell present in a well, and wherein the presence of the luminescent signal is indicative of an aspect of synaptic vesicle cycling in the neuronal cell.

In yet another aspect of the invention, a neuronal cell culture platform for analyzing an aspect of synaptic vesicle cycling is provided, the platform including a) a plurality of wells, wherein each well comprises a plurality of neuronal cells, and wherein a plurality of the neuronal cells comprise a reporter molecule attached to a vesicle protein; b) a stimulator system configured to induce synaptic vesicle cycling in neuronal cells present in the wells; and c) a detection system comprising an objective lens configured to collect luminescent signal from a reporter molecule attached to a synaptic vesicle protein of a neuronal cell present in a well, and wherein the presence of the luminescent signal is indicative of an aspect of synaptic vesicle cycling in the neuronal cell. In certain embodiments, the stimulator system comprises a plurality of electrode pairs, wherein each electrode pair is positioned within one of the plurality of wells, and wherein each electrode pair is configured to produce an electric field that is sufficient to induce synaptic vesicle cycling in a neuronal cell present in the well. In some embodiments of any of the aforementioned aspects of the invention, the synaptic vesicle protein comprises a lumenal portion. In certain embodiments of any of the aforementioned aspects of the invention, the objective lens is an oil or water objective lens. In some embodiments of any of the aforementioned aspects of the invention, the objective lens is an air objective lens. In certain embodiments of any of the aforementioned aspects of the invention, the objective lens is operably linked to a optical detector. In some embodiments of any of the aforementioned aspects of the invention, the optical detector is a charged-coupled device camera. In some embodiments of any of the aforementioned aspects of the invention, the platform is configured for detecting luminescent signal from a reporter molecule attached to a synaptic vesicle protein of a neuronal cell that has been stimulated to produce at least 5 action potentials. In certain embodiments of any of the aforementioned aspects of the invention, the detection system comprises a plurality of objective lenses. In some embodiments of any of the aforementioned aspects of the invention, the plurality of neuronal cells in a well is in a range of 10 to 100,000 neuronal cells. In some embodiments of any of the aforementioned aspects of the invention, the plurality of neuronal cells in a well is in a range of 1000 to 4000 cells/mm² of well bottom area. In certain embodiments of any of the aforementioned aspects of the invention, the plurality of neuronal cells comprise at least two different neuronal cell types. In some embodiments of any of the aforementioned aspects of the invention, the neuronal cells are primary neurons, optionally wherein the primary neurons are rat primary neurons. In some embodiments of any of the aforementioned aspects of the invention, the neuronal cells are selected from the group consisting of: glutamatergic, GABAergic, dopaminergic, adrenergic, serotonergic, and cholinergic neuronal cells. In certain embodiments of any of the aforementioned aspects of the invention, the neuronal cells comprise a transgene that expresses a synaptic vesicle protein, having a lumenal portion, fused to a reporter molecule. In some embodiments of any of the aforementioned aspects of the invention, each electrode of an electrode pair has a substantially curvilinear surface. In some embodiments of any of the aforementioned aspects of the invention, the electrodes of each electrode pair are substantially concentric cylinders, and wherein the concentric cylinders are separated by an annular insulating material. In certain embodiments of any of the aforementioned aspects of the invention, the platform also includes an electrode transfer system configured to operably position each electrode pair of the plurality of electrode pairs into one well of the plurality of wells. In some embodiments of any of the aforementioned aspects of the invention, the platform also includes a power source operably linked to the plurality of electrodes. In some embodiments of any of the aforementioned aspects of the invention, the power source is configured to apply a predetermined voltage across each electrode pair. In certain embodiments of any of the aforementioned aspects of the invention, the voltage is in range of 1 V to 400 V. In some embodiments of any of the aforementioned aspects of the invention, the voltage is in a range of 5 V to 20 V. In some embodiments of any of the aforementioned aspects of the invention, the platform also includes a pulse generator operably linked to the power source and the plurality of electrode pairs, wherein the pulse generator is configured to apply a predetermined voltage pulse across each electrode pair. In some embodiments of any of the aforementioned aspects of the invention, the pulse generator is configured to apply a plurality of predetermined voltage pulses at a predetermined frequency for a predetermined time. In certain embodiments of any of the aforementioned aspects of the invention, the predetermined frequency is in a range of 0.2 Hz to 100 Hz. In some embodiments of any of the aforementioned aspects of the invention, the predetermined frequency is in a range of 10 Hz to 50 Hz. In some embodiments of any of the aforementioned aspects of the invention, the predetermined time is up to 2 minutes. In certain embodiments of any of the aforementioned aspects of the invention, wherein the predetermined time is in a range of 0.1 to 20 seconds. In some embodiments of any of the aforementioned aspects of the invention, the predetermined time is in a range of 5 to 15 seconds. In some embodiments of any of the aforementioned aspects of the invention, the duration of each pulse is in a range of up to 10 msec. In certain embodiments of any of the aforementioned aspects of the invention, the duration of each pulse is in a range of 0.1 msec to 2 msec. In some embodiments of any of the aforementioned aspects of the invention, the duration between the initiation of each pulse is in a range of 0.1 to 5 msec. In some embodiments of any of the aforementioned aspects of the invention, the number of pulses is in a range of 1 to 1000. In some embodiments of any of the aforementioned aspects of the invention, the platform also includes a computer operably linked to the pulse generator, wherein the computer is configured to control the voltage pulse. In certain embodiments of any of the aforementioned aspects of the invention, the detection system comprises an optical sensor. In some embodiments of any of the aforementioned aspects of the invention, the detection system comprises an objective lens configured to collect a luminescent signal from a well. In some embodiments of any of the aforementioned aspects of the invention, the objective lens is configured to collect a luminescent signal from a field area in a range of 0.2 mm to 5 mm. In certain embodiments of any of the aforementioned aspects of the invention, the objective lens has a numerical aperture in a range 0.4 to 1.4. In some embodiments of any of the aforementioned aspects of the invention, the objective lens has a numerical aperture of 0.5. In some embodiments of any of the aforementioned aspects of the invention, the objective lens is not an oil or water immersion lens. In certain embodiments of any of the aforementioned aspects of the invention, the detection system comprises a plurality of detectors configured to simultaneously detect signals from a plurality of wells. In some embodiments of any of the aforementioned aspects of the invention, the detection system comprises a computer operably linked to the detectors, and wherein the computer is configured to transform luminescent signal from the detectors into data characterizing an aspect of synaptic vesicle cycling in a neuronal cell. In certain embodiments of any of the aforementioned aspects of the invention, each detector is configured to detect a luminescent signal from a plurality of reporter molecules. In some embodiments of any of the aforementioned aspects of the invention, each detector is configured to detect a luminescent signal from a plurality of synapses. In some embodiments of any of the aforementioned aspects of the invention, each detector is configured to detect a luminescent signal from a plurality of neuronal cells. In certain embodiments of any of the aforementioned aspects of the invention, the synaptic vesicle protein is VAMP2, vGlut1, synaptophysin, vesicular GABA transporter; acetylcholine transporter, catecholamine transporter or synaptotagmin. In some embodiments of any of the aforementioned aspects of the invention, the reporter molecule is attached to the lumenal portion of the synaptic vesicle protein. In some embodiments of any of the aforementioned aspects of the invention, the reporter molecule is a pH sensitive fluorescent protein. In certain embodiments of any of the aforementioned aspects of the invention, the reporter molecule is a pHluorin. In some embodiments of any of the aforementioned aspects of the invention, the reporter molecule comprises a sequence set forth in SEQ ID NO: 1 (hSyn-SypHy). In certain embodiments of any of the aforementioned aspects of the invention, at a pH in a range of 7.0 to 8.0, the pH sensitive reporter fluoresces with an intensity that is significantly greater than at a pH in a range of 5.0 to 6.0. In some embodiments of any of the aforementioned aspects of the invention, at a pH in a range of 5.0 to 6.0, the pH sensitive reporter fluoresces with an intensity that is significantly greater than at a pH in a range of 7.0 to 8.0. In certain embodiments of any of the aforementioned aspects of the invention, the luminescent signal is a fluorescent signal in a range of 475 nm to 525 nm.

According to another aspect of the invention, methods of measuring an aspect of synaptic vesicle cycling in a plurality of cells are provided. The methods include a) providing in each of a plurality of wells, an electrode pair and a plurality of cells expressing a fluorescent reporter molecule associated with a synaptic vesicle protein; b) inducing, with the electrode pairs, a series of action potentials in the plurality of cells sufficient to trigger synaptic vesicle cycling in the cells; and c) detecting a luminescent signal of the reporter molecule in the plurality of wells; wherein the a luminescent signal of the reporter molecule is a measure of an aspect of synaptic vesicle cycling. In some embodiments, the plurality of cells are neuronal cells. In some embodiments, the plurality of neuronal cells in a well is in a range of 10 to 100000 neuronal cells. In certain embodiments, the plurality of neuronal cells in a well is in a range of 1000 to 2000 cells/mm² of well bottom area. In some embodiments, the plurality of cells comprise at least two different neuronal cell types. In some embodiments, the neuronal cells are primary neurons, optionally wherein the primary neurons are rat primary neurons. In certain embodiments, the neuronal cells are selected from the group consisting of: glutamatergic, GABAergic, dopaminergic, adrenergic, serotonergic, and cholinergic neuronal cells. In some embodiments, a plurality of the neuronal cells express a synaptic vesicle protein fused to a reporter molecule. In some embodiments, each electrode of an electrode pair has a substantially curvilinear surface. In certain embodiments, the electrodes of each electrode pair are substantially concentric cylinders, and wherein the concentric cylinders are separated by an annular insulating material. In some embodiments, the method also includes positioning each electrode pair of the plurality of electrode pairs into one well of the plurality of wells, with an electrode transfer system. In certain embodiments, the action potentials are induced by a power source operably linked to the plurality of electrodes. In some embodiments, the action potentials are induced by applying a predetermined voltage across each electrode pair. In some embodiments, the voltage is in a range of 1 V to 400 V. In certain embodiments, the voltage is in a range of 5 V to 20 V. In some embodiments, a pulse generator is operably linked to the power source and the plurality of electrode pairs, and wherein the pulse generator applies the predetermined voltage pulse across each electrode pair. In some embodiments, the pulse generator applies a plurality of predetermined voltage pulses at a predetermined frequency for a predetermined time. In some embodiments, the predetermined frequency is in a range of 0.2 Hz to 100 Hz. In certain embodiments, the predetermined frequency is in a range of 10 Hz to 50 Hz. In some embodiments, the predetermined time is less than or equal to 2 minutes. In some embodiments, the predetermined time is in a range of 0.1 to 20 seconds. In some embodiments, the predetermined time is in a range of 5 to 15 seconds. In certain embodiments, the duration of each pulse is in a range of up to 10 msec. In some embodiments, the duration of each pulse is in a range of 0.1 msec to 2 msec. In some embodiments, the duration between the initiation of each pulse in a range of 0.1 to 5 msec. In some embodiments, the number of pulses is in a range of 1 to 1000. In certain embodiments, the voltage pulse is controlled by a computer operably linked to the pulse generator, wherein the computer is configured to control the voltage pulse. In some embodiments, the luminescent signal is detected by a detector. In some embodiments, the detector comprises an optical sensor. In certain embodiments, the luminescent signal of the reporter molecule is detected using a plurality of detectors, wherein a charge-coupled device camera is operably linked to each detector. In some embodiments, each detector comprises an objective configured to collect a luminescent signal from a well. In some embodiments, the objective is configured to collect a luminescent signal from a field area in a range of 0.2 mm to 5 mm. In certain embodiments, the objective has a numerical aperture in a range 0.4 to 1.4. In some embodiments, the objective has a numerical aperture of 0.5. In some embodiments, the plurality of detectors simultaneously detects signals from a plurality of wells. In some embodiments, a computer is operably linked to the plurality of detectors and transforms the luminescent signal from the detectors into data characterizing an aspect of synaptic vesicle cycling in a neuronal cell. In certain embodiments, each detector detects a luminescent signal from a plurality of reporter molecules. In some embodiments, each detector detects a luminescent signal from a plurality of synapses. In some embodiments, each detector detects a luminescent signal from a plurality of neuronal cells. In some embodiments, the synaptic vesicle protein is VAMP2, vGlut1, synaptophysin, vesicular GABA transporter; acetylcholine transporter, catecholamine transporter or synaptotagmin. In certain embodiments, the synaptic vesicle protein has a lumenal portion. In some embodiments, the luminescent reporter molecule is attached to the lumenal portion. In some embodiments, the luminescent reporter molecule is a pH sensitive reporter. In some embodiments, the luminescent reporter molecule is a pHluorin. In certain embodiments, the luminescent reporter molecule comprises a sequence set forth in SEQ ID NO: 1 (hSyn-SypHy). In some embodiments, at a pH in a range of 7.0 to 8.0, the pH sensitive reporter fluoresces with an intensity that is significantly greater than at a pH in a range of 5.0 to 6.0. In some embodiments, at a pH in a range of 7.0 to 8.0, the pH sensitive reporter fluoresces with an intensity that is significantly greater than at a pH in a range of 7.0 to 8.0. In certain embodiments, the luminescent signal is in a range of 475 nm to 525 nm. In some embodiments, the method also including d) contacting the plurality of cells in the plurality of wells with at least one test agent to be tested for its ability to modulate an aspect of synaptic vesicle cycling; e) inducing a second series of action potentials in the cells sufficient to trigger synaptic vesicle cycling in the cells; f) detecting a second luminescent signal of the reporter molecule in the plurality of wells; wherein a significant difference between the luminescent signal detected in step (c) and the luminescent signal detected in step (f) identifies the test agent as modulating an aspect of synaptic vesicle cycling. In some embodiments, the method also includes contacting, prior to step (b), the plurality of cells in the plurality of wells with at least one test agent to be tested for its ability to modulate an aspect of synaptic vesicle cycling; wherein a significant difference between the luminescent signal detected in step (c) and a control luminescent signal identifies the test agent as modulating an aspect of synaptic vesicle cycling. In certain embodiments, the method also includes d) contacting the plurality of cells in at least one well of the plurality of wells with at least one test agent to be tested for its ability to modulate an aspect of synaptic vesicle cycling; e) contacting the plurality of cells in at least one well of the plurality of wells with at least one control agent; wherein a significant difference between the luminescent signal detected in a well having a test agent and the luminescent signal detected in a well having a control agent identifies the test agent as modulating an aspect of synaptic vesicle cycling. In some embodiments, the method also includes d) contacting the plurality of cells in at least one well of the plurality of wells with at least one test agent to be tested for its ability to modulate an aspect of synaptic vesicle cycling; wherein a significant difference between the luminescent signal detected in a well having a test agent and the luminescent signal detected in a negative control well identifies the test agent as modulating an aspect of synaptic vesicle cycling. In some embodiments, the method also includes contacting the plurality of cells in the negative control well with a control agent that does not modulate an aspect of synaptic vesicle cycling. In certain embodiments, the method also includes d) contacting the plurality of cells in at least one well of the plurality of wells with at least one test agent to be tested for its ability to modulate an aspect of synaptic vesicle cycling; wherein no significant difference between the luminescent signal detected in a well having a test agent and the luminescent signal detected in a positive control well identifies the test agent as modulating an aspect of synaptic vesicle cycling. In some embodiments, the method also includes contacting the plurality of cells in the positive control well with a control agent that modulates an aspect of synaptic vesicle cycling. In certain embodiments, the test agent is a small molecule. In some embodiments, the test agent is a polypeptide. In certain embodiments, the test agent is an antibody. In some embodiments, the test agent is a nucleic acid. In some embodiments, the nucleic acid is selected a DNA, RNA, DNA/RNA hybrid, short interfering RNA, short hairpin RNA, micro RNA, ribozyme, or aptamer. In certain embodiments, the test agent is a carbohydrate. In some embodiments, the test agent is a lipid. In some embodiments, the lipid is a phospholipid, triglyceride, or steroid. In certain embodiments, the method also includes monitoring the toxicity of a test agent identified as a modulator of an aspect of synaptic vesicle cycling in an in vivo model. In some embodiments, the method also includes monitoring the efficacy of a test agent identified as a modulator of an aspect of synaptic vesicle cycling in an in vivo model. In some embodiments, the method is a high-throughput screening process. In some embodiments, the luminescent signal is a level of fluorescence. In certain embodiments, the luminescent signal is a plurality of fluorescence levels obtained over a predefined time. In some embodiments, the luminescent signal is a rate of rise of fluorescence. In some embodiments, the luminescent signal is a rate of decay of fluorescence.

According to yet another aspect of the invention, methods of identifying a test agent as a modulator of an aspect of synaptic vesicle cycling are provided. The methods include a) providing in a plurality of wells, each well comprising an electrode pair, and a plurality of cells expressing a fluorescent reporter molecule associated with a synaptic vesicle protein; b) inducing a first series of action potentials in the plurality of cells sufficient to trigger synaptic vesicle cycling in the cells; c) detecting a first luminescent signal of the reporter molecule in the plurality of wells; d) contacting the plurality of cells in the plurality of wells with at least one test agent to be tested for its ability to modulate an aspect of synaptic vesicle cycling; e) inducing a second series of action potentials in the cells sufficient to trigger synaptic vesicle cycling in the cells; and f) detecting a second luminescent signal of the reporter molecule in the plurality of wells; wherein a significant difference between the first and second levels of fluorescence of the reporter molecule identifies the test agent as a modulator of an aspect of synaptic vesicle cycling.

According to another aspect of the invention, methods of measuring an aspect of synaptic vesicle cycling in a plurality of cells are provided. The methods including a) providing in each of a plurality of wells, an electrode pair and a plurality of cells expressing a fluorescent reporter molecule associated with a synaptic vesicle protein; b) inducing, with the electrode pairs, a series of action potentials in the plurality of cells sufficient to trigger synaptic vesicle cycling in the cells; c) detecting a luminescent signal of the reporter molecule in the plurality of wells; wherein the a luminescent signal of the reporter molecule is a measure of an aspect of synaptic vesicle cycling; and d) contacting the plurality of cells in at least one well of the plurality of wells with at least one test agent to be tested for its ability to modulate an aspect of synaptic vesicle cycling; wherein a comparison between the luminescent signal detected in a well having a test agent and the luminescent signal detected in a control well identifies the test agent as modulating an aspect of synaptic vesicle cycling.

According to another aspect of the invention, methods for measuring an aspect of synaptic vesicle cycling in a plurality of cells are provided. The method including a) providing in each of a plurality of wells, a stimulator and a plurality of cells expressing a fluorescent reporter molecule associated with a synaptic vesicle protein; b) inducing, with the stimulator, a series of action potentials in the plurality of cells sufficient to trigger synaptic vesicle cycling in the cells; and c) detecting a luminescent signal of the reporter molecule in the plurality of wells; wherein the a luminescent signal of the reporter molecule is a measure of an aspect of synaptic vesicle cycling.

### BRIEF DESCRIPTION OF THE DRAWINGS

FIG. 1. Schematic diagram of the synaptic vesicle cycle, showing where certain presynaptic proteins are thought to function.

FIGS. 2A-2B. Elevational-view depiction of an electrode pair which may be used to excite synaptic vesicle cycling in neuronal cells. (FIG. 2A) The electrode pair comprises an inner rod-like electrode 102 and an outer cylindrically-shaped electrode 104. The electrode pair may be placed in contact with, or in close proximity to, a neuronal cell. (FIG. 2B) A top-view illustration of the electrode pair shown in (FIG. 2A).

FIGS. 3A-3D. Several embodiments of voltage waveforms that may be applied to the electrodes of FIGS. 2A-2B.

FIGS. 4A-4B. (FIG. 4A) An instantaneous electric field [symbol] which may occur between the electrodes of FIGS. 2A-2B in response to the application of voltages to the electrodes. (FIG. 4B) An instantaneous electric field [symbol] which may occur in the vicinity of a neuronal cell.

FIGS. 5A-5H. Illustrations of various embodiments of electrode configurations which may be used to excite synaptic vesicle cycling.

FIGS. 6A-6C. (FIGS. 6A-6B) An embodiment of an electrode assembly. The electrodes 102, 104 are supported by a plate 503, and electrical contact to the electrodes is made through conductive traces 510, 512. (FIG. 6C) An elevational-view photograph of an electrode assembly.

FIGS. 7A-7C. Illustrations of a multi-electrode assembly which may be used to excite, substantially simultaneously, synaptic vesicle cycling in multiwell plates. (FIG. 7A) top-view illustration; (FIG. 7B) elevation-view illustration; (FIG. 7C) bottom-view illustration.

FIGS. 8A-8B. (FIG. 8A) An embodiment of a multi-electrode assembly in which columns, or rows, of electrode pairs may be excited individually. (FIG. 8B) Localized electrical contacts 722A-722L for separate conductive traces 610.

FIG. 9. A block diagram of an embodiment of an electrical circuit which may be used to apply voltage waveforms to one or more electrode pairs.

FIG. 10. An elevation-view illustration depicting an embodiment of a synaptic vesicle cycling platform. The platform comprises a multiwell plate 915, an inverted microscope 910, positioning apparatus 945, and a multi-electrode assembly 600. In various embodiments, the microscope 910 images at least a portion of synaptic vesicle cycling activity occurring within a well 908.

FIG. 11. An elevation-view illustration depicting an embodiment of a synaptic vesicle cycling platform. The platform comprises a multiwell plate 915, a multi-electrode assembly 600, a lens array 1070, and a photodetector array 1080. In various embodiments, at least a portion of fluorescent radiation emitted during synaptic vesicle cycling within the wells is collected by lenses 1072 and directed to corresponding photodetectors 1082. This embodiment of the synaptic vesicle cycling platform provides for parallel monitoring of synaptic vesicle cycling in separate vessels, such as wells that comprise neuronal cells. The wells can be separate wells of a multiwell plate.

FIG. 12. Cypher5E (GE Healthcare) is a pH-sensitive dye which fluoresces at ˜pH 5.5 and is quenched at ˜pH 7.4. The differential fluorescence of Cypher5E at different pH occurs because it has a different structure at pH 5.5 (right) than it does at pH 7.4 (left).

FIGS. 13A-13B. Exemplary results illustrating the synaptic vesicle cycle at the whole well level in primary neurons infected with an adeno-associated virus that expresses synaptopHluorin measured on an inverted microscope (FIG. 13A) and on the plate::vision plate reader (FIG. 13B).

FIG. 14. Exemplary results illustrating the synaptic vesicle cycle at the whole well level in primary neurons infected with an adeno-associated virus that expresses synaptopHluorin measured on the plate::vision plate reader.

FIG. 15. Exemplary custom electrode including an outer platinum ring electrode with an outer diameter of 6 mm, which fits into the well of a 96 well plate.

FIGS. 16A-16D. Experimental results (fluorescence images and fluorescence signal traces) are shown. The data was obtained from the stimulation of action potentials in primary neurons. The neuronal cells were cultivated in 96-well plates, and stimulation was done with a modified, commercially-available electroporation system (Cellaxess® CX3, Cellectricon AB, Moldnal, Sweden). This result demonstrates that the Cellectricon system can stimulate action potentials in primary neurons as measured by Ca⁺⁺ imaging of primary neuronal cultures. Scale bar in this figure is 100 μm.

FIGS. 17A-17C. Experimental results (fluorescence images and a fluorescence signal trace) are shown from an experiment in which synaptic vesicle cycling was stimulated in 96-well plates. The electrical stimulation system was the same as that used for the experiment of FIGS. 16A-16D. This figure demonstrates that the Cellectricon system can stimulate synaptic vesicle cycling in primary neurons as measured by synaptopHluorin imaging of primary neuronal cultures.

FIG. 18. Exemplary results illustrating that the plate::vision plate reader can measure the synaptic vesicle cycle using the synaptophysin-pHluorin reporter when neurons are stimulated with the customized Cellectricon electrode.

FIG. 19. Exemplary results illustrating that the plate::vision plate reader system can successfully detect synaptophysin-pHluorin response to a small number of action potentials.

FIG. 20. Illustration of an exemplary high-content screening system.

FIG. 21. Exemplification of a presynaptic HTS platform instrument. (Top) Upper cabinet of the presynaptic HTS platform instrument, showing the location of the Tecan robot liquid handling head, the electrical stimulation tip module, the assay plate, and the iCCD camera from the plate::vision plate reader. (Bottom) Lower cabinet showing the location of the pulse generator, the plate reader, the plate reader lamp, and various power supplies.

FIG. 22. Depiction of results from 96 parallel presynaptic assays using the presynaptic HTS platform, with the traces normalized to their peak response.

FIGS. 23A-23C. Exemplary results from a uniformity analysis of the presynaptic HTS platform electrical stimulation system. (FIG. 23A) shows the sypHy fluorescence response to stimulation trains (gray bar on x-axis) from a single well. (FIG. 23B) shows the peak sypHy fluorescence amplitude as a function of stimulus voltage and the derivation of the EV50 measurement. (FIG. 23C) shows the EV50 as a function of well from across the 96-well plate. The electrical stimulation system delivers highly uniform current densities across 96-well plates.

FIG. 24. Exemplary results from a sensitivity analysis of the presynaptic HTS platform.

FIG. 25. Detection of compound-induced changes in the synaptic vesicle cycling with the presynaptic HTS platform.

FIG. 26. Measurement of the synaptic vesicle cycle using a high content imaging system.

FIG. 27. Exemplary results from an analysis of synaptic vesicle cycling using a Fluoroskan Ascent FL plate reader.

### DEFINITIONS

Agent: As used herein, the term “agent” (also referred to as “test agent” or “candidate agent”) refers to any compound or composition that can be tested as a potential modulator. Examples of agents that can be used include, but are not limited to, small molecules, antibodies, antibody fragments, siRNAs, shRNAs, nucleic acid molecules (RNAs, DNAs, or DNA/RNA hybrids), antisense oligonucleotides, ribozymes, peptides, peptide mimetics, carbohydrates, lipids, microorganisms, natural products, and the like. In some embodiments, an agent can be isolated or not isolated. As a non-limiting example, an agent can be a library of agents. If a mixture of agents is found to be a modulator, the pool can then be further purified into separate components to determine which components are in fact modulators of a target activity.

Amino acid: As used herein, the term “amino acid,” in its broadest sense, refers to any compound and/or substance that can be incorporated into a polypeptide chain. In some embodiments, an amino acid has the general structure H₂N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, and/or substitution with other chemical groups that can change the peptide's circulating half-life without adversely affecting their activity. Amino acids may participate in a disulfide bond. The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Antibody: As used herein, the term “antibody” refers to any immunoglobulin, whether natural or wholly or partially synthetically produced. All derivatives thereof which maintain specific binding ability are also included in the term. The term also covers any protein having a binding domain which is homologous or largely homologous to an immunoglobulin binding domain. Such proteins may be derived from natural sources, or partly or wholly synthetically produced. An antibody may be monoclonal or polyclonal. An antibody may be a member of any immunoglobulin class, including any of the human classes: IgG, IgM, IgA, IgD, and IgE. As used herein, the terms “antibody fragment” or “characteristic portion of an antibody” are used interchangeably and refer to any derivative of an antibody which is less than full-length. In general, an antibody fragment retains at least a significant portion of the full-length antibody's specific binding ability. Examples of antibody fragments include, but are not limited to, Fab, Fab′, F(ab′)2, scFv, Fv, dsFv diabody, and Fd fragments. An antibody fragment may be produced by any means. For example, an antibody fragment may be enzymatically or chemically produced by fragmentation of an intact antibody and/or it may be recombinantly produced from a gene encoding the partial antibody sequence. Alternatively or additionally, an antibody fragment may be wholly or partially synthetically produced. An antibody fragment may optionally comprise a single chain antibody fragment. Alternatively or additionally, an antibody fragment may comprise multiple chains which are linked together, for example, by disulfide linkages. An antibody fragment may optionally comprise a multimolecular complex. A functional antibody fragment typically comprises at least about 50 amino acids and more typically comprises at least about 200 amino acids.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. In some embodiments, the moieties are attached to one another by one or more covalent bonds. In some embodiments, the moieties are attached to one another by a mechanism that involves non-covalent binding (e.g. hydrogen bonding, affinity interactions, electrostatic interactions, Van der Waals forces, etc.). In some embodiments, a sufficient number of weaker interactions can provide sufficient stability for moieties to remain physically associated.

Biocompatible: As used herein, the term “biocompatible” refers to substances that are not toxic to cells. In some embodiments, a substance is considered to be “biocompatible” if its addition to cells in vivo does not induce inflammation and/or other adverse effects in vivo. In some embodiments, a substance is considered to be “biocompatible” if its addition to cells in vitro or in vivo results in less than or equal to about 50%, about 45%, about 40%, about 35%, about 30%, about 25%, about 20%, about 15%, about 10%, about 5%, or less than about 5% cell death.

Biodegradable: As used herein, the term “biodegradable” refers to substances that are degraded under physiological conditions. In some embodiments, a biodegradable substance is a substance that is broken down by cellular machinery. In some embodiments, a biodegradable substance is a substance that is broken down by chemical processes.

Control: As used herein, the term “control” has its art-understood meaning of being a standard against which results are compared. Typically, controls are used to augment integrity in experiments by isolating variables in order to make a conclusion about such variables. In some embodiments, a control is a reaction or assay that is performed simultaneously with a test reaction or assay to provide a comparator. In one experiment, the “test” (i.e., the variable being tested) is applied. In the second experiment, the “control,” the variable being tested is not applied. In some embodiments, a control is a historical control (i.e., of a test or assay performed previously, or an amount or result that is previously known). In some embodiments, a control is or comprises a printed or otherwise saved record. A control may be a positive control or a negative control.

Detection System: A detection system, as used herein, is a system for monitoring an aspect of synaptic vesicle cycling. A detection system typically provides monitoring of a luminescent signal from at least a region within a well comprising neuronal cells. A detection system may provide simultaneous monitoring of luminescent signals, each luminescent signal from at least a region within a well comprising neuronal cells, from a plurality of wells. A detection system may comprise a detector, e.g., optical sensor, objective lens, photomultiplier tube, etc., configured to detect luminescent signals from reporter molecules in neuronal cells within a well. A detection system may comprise a plurality of detectors, e.g., optical sensors, objective lenses, etc., each configured to detect luminescent signals from reporter molecules in neuronal cells within a well. A detection system typically comprises a photosensitive component configured to convert luminescent signals into a digital electrical signals. For example, a detection system may comprise a charged coupled system camera operably linked to detectors. A detection system may be operably linked to a computer configured to control the detection of luminescent signals.

Dephosphins: As used herein, the term “dephosphins” refers to proteins that, when dephosphorylated, regulate (e.g., enhance the efficiency of) synaptic vesicle endocytosis. Typically, the dephosphins, when dephosphorylated, are assembled into a protein complex. In some embodiments, dephosphins are dephosphorylated by calcineurin.

Dysfunction: As used herein, the term “dysfunction” refers to an abnormal function of a molecule or a process. Dysfunction of a molecule (e.g., a protein) can be caused by an increase or decrease of an activity associated with such molecule. Dysfunction of a molecule can be caused by defects associated with the molecule itself or other molecules that directly or indirectly interacting with or regulating the molecule. Dysfunction of a process (e.g., synaptic vesicle cycling) can be caused by an increase or decrease of an activity associated with any molecule directly involved in the process or other molecules or processes that directly or indirectly interacting with or regulating such molecule.

Electrical Stimulation System: As used herein, the term “electrical stimulation system” refers to an apparatus comprising at least one electrode assembly which may be placed in contact with, or in close proximity to, a neuronal cell culture, suspension, and/or preparation. The electrical stimulation system may optionally further comprise electrical equipment which produces voltage or current waveforms.

Electrode assembly: As used herein, the term “electrode assembly” refers to an apparatus which supports and includes an electrode pair. The apparatus may further include conductive wires or traces connecting to each of the electrodes in the electrode pair.

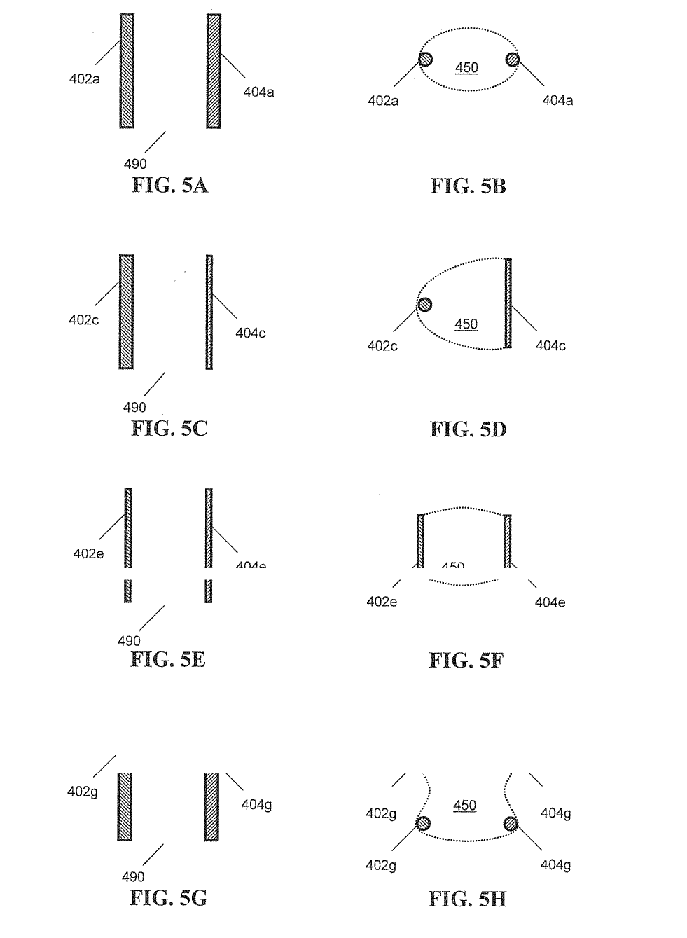

Electrode pair: As used herein, the term “electrode pair” generally refers to two conductive elements configured such that one element functions as a cathode and one element functions as an anode. In certain embodiments, see for example FIG. 5H, the term “electrode pair” may be applied to more than two conductive elements wherein some of the elements are configured to function as cathodes and some of the elements are configured to function as anodes.

Excitation region: As used herein, the term “excitation region” refers to a region within a cell culture, suspension, and/or preparation subjected to a stimulation waveform. The stimulation waveform may excite synaptic vesicle cycling in neuronal cells within this region.

Functional: As used herein, a “functional” biological molecule or process is a biological molecule or process in a form in which it exhibits a property and/or activity by which it is characterized.

Instantaneous electric field: As used herein, the term “instantaneous electric field” is used to describe a snap-shot of a time-varying electric field. For example, instantaneous electric field is a representation of a time-varying electric field at an instant in time.

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism such as a non-human animal.

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from at least about 10%, about 20%, about 30%, about 40%, about 50%, about 60%, about 70%, about 80%, about 90%, about 95%, about 98%, about 99%, substantially 100%, or 100% of the other components with which they were initially associated. In some embodiments, isolated agents are more than about 80%, about 85%, about 90%, about 91%, about 92%, about 93%, about 94%, about 95%, about 96%, about 97%, about 98%, about 99%, substantially 100%, or 100% pure. As used herein, a substance is “pure” if it is substantially free of other components. As used herein, the term “isolated cell” refers to a cell not contained in a multi-cellular organism. It is to be appreciated that an isolated cell may have previously been present in vivo, e.g., a primary cell.

Luminescent signal: As used herein, the term “luminescent signal” refers to a quantity of light emitted from at least one reporter molecule or an electrical signal, e.g., digital electrical signal, representative of a quantity of light emitted from at least one reporter molecule. A luminescent signal may be a level of luminescence intensity, e.g., fluorescence intensity, e.g., a maximum intensity level, from at least one reporter molecule. A luminescent signal may be a plurality of levels of luminescence intensity, e.g., fluorescence intensity, from at least one reporter molecule occurring over a predetermined time. A luminescent signal may be a rate of rise or decay of levels of luminescence, e.g., fluorescence, intensity from at least one reporter molecule over a predetermined time.

Modulator: As used herein, the term “modulator” refers to an agent, e.g., a compound, that alters or elicits an activity. For example, the presence of a modulator may result in an increase or decrease in the magnitude of a certain activity compared to the magnitude of the activity in the absence of the modulator. In certain embodiments, a modulator is an inhibitor, which decreases the magnitude of one or more activities. In certain embodiments, an inhibitor completely prevents one or more biological activities. In certain embodiments, a modulator is an activator, which increases the magnitude of at least one activity. In certain embodiments the presence of a modulator results in an activity that does not occur in the absence of the modulator.

Multi-electrode assembly: As used herein, the term “multi-electrode assembly” refers to an apparatus which supports and includes plural electrode pairs. The apparatus may optionally further include conductive wires or traces connecting to each of the electrodes in the multi-electrode assembly.

Neuronal cell: As used herein, the term “neuronal cell” (also referred to as a “neuron”) refers to a cell that undergoes synaptic vesicle cycling or one or more aspects of synaptic vesicle cycling. In some embodiments, a neuronal cell has or can form presynaptic terminals, wherein the presynaptic terminals have a functional synaptic vesicle cycle or undergo one or more activities of synaptic vesicle cycling. In some embodiments, a neuronal cell is in vitro. In some embodiments, a neuronal cell is in vivo. In some embodiments, a neuronal cell is in a culture of growing cells, a suspension of cells, a plurality of cells associated with (e.g., grown on, affixed to, tethered to, etc.) a surface, a plurality of neuronal cells that have been substantially purified from an in vivo source (e.g., harvested from an animal), and/or a cell line. In some embodiments, a neuronal cell is within a live animal (e.g., rodent, human, etc.). In some embodiments, a neuronal cell is a primary neuronal cell and/or a stem cell. In some embodiments, neuronal cells are primary rat forebrain neurons. Neuronal cells may comprise collections of cells which undergo one or more elements of presynaptic vesicle cycling. In some embodiments, neuronal cells can be transformed, transfected, infected, and/or otherwise induced to take up a desired reporter for tracking synaptic vesicle cycling or one or more activities thereof. In some embodiments, neuronal cells may be utilized in synaptic vesicle cycling assays in accordance with the invention.

Neuronal cell system: As used herein, the term “neuronal cell system” (also referred to as “neuronal system”) refers to a system that undergoes synaptic vesicle cycling or one or more activities of synaptic vesicle cycling. In some embodiments, a neuronal system comprises at least one cell which has or can form presynaptic terminals, wherein the presynaptic terminals have a functional synaptic vesicle cycle or undergo one or more activities of synaptic vesicle cycling. In some embodiments, a neuronal cell system is an in vitro system. In some embodiments, a neuronal cell system is an in vivo system. In some embodiments, a neuronal cell system is an in vitro system comprising a culture of growing cells, a suspension of cells, a plurality of cells associated with (e.g., grown on, affixed to, tethered to, etc.) a surface, a plurality of neuronal cells that have been substantially purified from an in vivo source (e.g., harvested from an animal), and/or a cell line. In some embodiments, a neuronal cell system comprises neuronal cells within a live animal (e.g., rodent, human, etc.). In some embodiments, neuronal cell systems comprise primary neuronal cultures and/or stem cells. In some embodiments, neuronal cell systems may comprise primary rat forebrain neurons. Neuronal cell systems may comprise collections of cells which undergo one or more elements of presynaptic vesicle cycling. In some embodiments, cells within a neuronal cell system can be transformed, transfected, infected, and/or otherwise induced to take up a desired reporter for tracking synaptic vesicle cycling or one or more activities thereof. In some embodiments, neuronal cell systems may be utilized in synaptic vesicle cycling assays in accordance with the invention.

Nucleic acid: As used herein, the term “nucleic acid,” in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising individual nucleic acid residues. As used herein, the terms “oligonucleotide” and “polynucleotide” can be used interchangeably. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention. The term “nucleotide sequence encoding an amino acid sequence” includes all nucleotide sequences that are degenerate versions of each other and/or encode the same amino acid sequence. Nucleotide sequences that encode proteins and/or RNA may include introns. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, backbone modifications, etc. A nucleic acid sequence is presented in the 5′ to 3′ direction unless otherwise indicated. The term “nucleic acid segment” is used herein to refer to a nucleic acid sequence that is a portion of a longer nucleic acid sequence. In many embodiments, a nucleic acid segment comprises at least 3, at least 4, at least 5, at least 6, at least 7, at least 8, at least 9, at least 10, or more residues. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, the present invention may be specifically directed to “unmodified nucleic acids,” meaning nucleic acids (e.g. polynucleotides and residues, including nucleotides and/or nucleosides) that have not been chemically modified in order to facilitate or achieve delivery.

Presynaptic proteins: As used herein, the term “presynaptic proteins” refers to proteins preferentially localized at presynaptic terminals. Typically, presynaptic proteins are involved in synaptic vesicle cycling. In some embodiments, presynaptic proteins include synapsins, dephosphins and other calcineurin substrates. Exemplary presynaptic proteins include, but are not limited to, synaptic vesicle proteins, also referred to herein as intrinsic vesicle proteins, (e.g., synaptic vesicle protein 2 (SV2), vesicular GABA transporter, acetylcholine transporter, catecholamine transporter, synaptophysins, synaptotagmins, vesicle-associated membrane polypeptides (VAMPs), neurotransmitter transporters (NT transporters), synaptogyrins, proton pump), peripheral vesicle proteins (e.g., Rabs, cystine string proteins (CSPs), synaptic plasma membrane proteins (e.g., calcium channels, synaptosome-associated protein of 25 kDa (SNAP-25), syntaxin), cytosolic proteins (e.g., SNAPs, n-Sec1), synapsins (e.g., synapsin I, II and III), and dephosphins (e.g., dynamin I, PIP5K1γ, and synaptojanin I).

Plurality: As used herein the term “plurality” or “plural” refers to more than one.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, etc. In some embodiments, proteins may comprise natural amino acids, non-natural amino acids, synthetic amino acids, and combinations thereof. The term “peptide” is generally used to refer to a polypeptide having a length of less than about 100 amino acids.

Reporter: As used herein, the term “reporter” (also referred to herein as a reporter molecule) refers to any substance which, when included in a neuronal cell, provides a detectable signal, e.g., a luminescent signal, e.g., a fluorescence signal, indicative of at least one activity of synaptic vesicle cycling. In some embodiments, a reporter is introduced into a neuronal cell by viral infection. In some embodiments, a reporter is added externally to a neuronal culture, suspension, and/or preparation. In some embodiments, a neuronal cell is derived from a transgenic animal expressing a reporter gene. In some embodiments, a reporter is a fluorophore. In some embodiments, a reporter is a dye.

Small molecule: In general, a “small molecule” is understood in the art to be an organic molecule that is less than about 5 kilodaltons (Kd) in size. In some embodiments, the small molecule is less than about 4 Kd, about 3 Kd, about 2 Kd, or about 1 Kd. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, small molecules are non-polymeric. In some embodiments, small molecules are not proteins, peptides, or amino acids. In some embodiments, small molecules are not nucleic acids or nucleotides. In some embodiments, small molecules are not saccharides or polysaccharides.

Stimulation System: As used herein, the term “stimulation system” refers to any system or composition that triggers synaptic vesicle cycling. Typically, a stimulation system triggers synaptic vesicle cycling by initiating action potentials in a neuronal cell. A stimulation system suitable for the invention can be an electrical stimulation system, an acoustic or ultrasonic stimulation system, an optical stimulation system or a biochemical stimulation system.

Stimulation waveform: As used herein, the term “stimulation waveform” generally refers to a time-varying stimulus applied to neuronal cells in culture, suspension, and/or otherwise prepared. For example, the stimulus may be an electric field characterized by a magnitude and direction which may both vary over time. A stimulation waveform may be produced by one or more voltage waveforms or current waveforms applied to electrodes in an electrical stimulation system.

Subject: As used herein, the term “subject” or “patient” refers to any organism to which compositions in accordance with the invention may be administered, e.g., for experimental, diagnostic, prophylactic, and/or therapeutic purposes. Typical subjects include animals (e.g., mammals such as mice, rats, rabbits, non-human primates, and humans; insects; worms; etc.).

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Susceptible to: An individual who is “susceptible to” a disease, disorder, and/or condition has not been diagnosed with the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Synaptic vesicle cycling modulator: As used herein, the term “synaptic vesicle cycling modulator” refers to any substance that alters any activity of a synaptic vesicle cycle. In some embodiments, relevant activities of the synaptic vesicle cycle include mobilization of synaptic vesicles to the presynaptic membrane, docking at the presynaptic membrane, priming for fusion with the presynaptic membrane, Ca²⁺ sensing, fusion with the presynaptic membrane, release of neurotransmitters into the synaptic cleft, retrieval of synaptic vesicles in the presynaptic terminal by endocytosis (which involves steps of clathrin-mediated nucleation, invagination of the presynaptic membrane, fission of the synaptic vesicle from the presynaptic membrane, and removal of clathrin from the endocytosed synaptic vesicles), translocation of synaptic vesicles within the presynaptic terminal, sorting of synaptic vesicles within the presynaptic terminal, and loading of synaptic vesicle lumen with neurotransmitter. In some embodiments, a synaptic vesicle cycling modulator enhances one or more activities of the synaptic vesicle cycle. In some embodiments, a synaptic vesicle cycling modulator inhibits one or more activities of the synaptic vesicle cycle. Exemplary synaptic vesicle cycling proteins include, e.g., synaptic vesicle protein 2 (SV2), vesicular GABA transporter, acetylcholine transporter, catecholamine transporter, synaptophysins, synaptotagmins, vesicle-associated membrane polypeptides (VAMPs), neurotransmitter transporters (NT transporters), and synaptogyrins, and proton pumps. Other synaptic vesicle cycling proteins will be apparent to the skilled artisan.

Synaptic vesicle cycling platform: As used herein, the term “synaptic vesicle cycling platform” refers to an apparatus which comprises at least a stimulation system (e.g., an electronic stimulation system) and detection system. In some embodiments, the electrical stimulation system delivers stimulation waveforms to neuronal cells in culture, suspension, and/or otherwise prepared. In some embodiments, the detection system monitors activity relating to synaptic vesicle cycling. In some embodiments, a synaptic vesicle cycling platform is utilized to carry out synaptic vesicle cycling assay(s) (e.g., assays to identify modulators of synaptic vesicle cycling). In some embodiments, the synaptic vesicle cycling platform can be operated by a single operator.

Synaptic vesicle protein: A synaptic vesicle protein is a presynaptic protein that associates with a synaptic vesicle. A synaptic vesicle protein typically has a transmembrane portion and may have a lumenal portion and/or a cytoplasmic portion. A lumenal portion of a presynaptic protein is a domain of the protein that is exposed to the lumen of a synaptic vesicle. It is to be understood that when a synaptic vesicle fuses with a synaptic membrane, e.g., during exocytosis of a synaptic vesicle cycle, a lumenal portion of a synaptic vesicle protein may become exposed to the extracellular space. A cytoplasmic portion of a presynaptic protein is a domain of the protein that is exposed to the cytoplasm of the cell in which the synaptic vesicle is present. It is to be understood that when a synaptic vesicle fuses with a synaptic membrane, e.g., during exocytosis of a synaptic vesicle cycle, a lumenal portion of a synaptic vesicle protein may remain exposed to the cytoplasm. A transmembrane portion of a presynaptic protein is a domain of the protein that is embedded within the vesicle membrane. It is to be understood that when a synaptic vesicle fuses with a synaptic membrane, e.g., during exocytosis of a synaptic vesicle cycle, a transmembrane portion of a synaptic vesicle protein becomes embedded in the cellular membrane.

Therapeutic agent: As used herein, the phrase “therapeutic agent” refers to any agent that, when administered to a subject, has a therapeutic effect and/or elicits a desired biological and/or pharmacological effect.

Time-varying electric field: As used herein, the term “time-varying electric field” is used to describe an electric field having a magnitude and direction which can change with time.

Waveform: As used herein, the term “waveform” is generally used to refer to various time-varying physical quantities, e.g., time-varying voltage, time-varying current, time-varying electric field, time-varying fluorescent emission, etc.

### DETAILED DESCRIPTION OF CERTAIN EMBODIMENTS OF THE INVENTION

The present invention provides systems and methods for identifying synaptic vesicle cycling modulators. Among other things, the present invention provides synaptic vesicle cycling assays. In some embodiments, synaptic vesicle cycling assays are utilized to monitor kinetics, efficiency, and/or other characteristics of the synaptic vesicle cycle. In some embodiments, synaptic vesicle cycling assays are utilized to identify substances that affect (e.g., stimulate and/or inhibit) at least one aspect of a synaptic vesicle cycle.

Synaptic vesicle cycling assays typically involve cells or cell cultures comprising presynaptic terminals which exhibit functional synaptic vesicle cycling or at least one activity thereof, a reporter (e.g., a reporter useful for tracking synaptic vesicle cycling or at least one activity thereof), a stimulation system (e.g., an electrical, acoustic, ultrasonic, optical stimulation system to trigger synaptic vesicle cycling, for example, via action potential initiation), and a detection system (e.g., an imaging apparatus to capture signal generated by the reporter). In some embodiments, synaptic vesicle cycling assays can be constructed and/or performed in a high-throughput format.

### Synaptic Vesicle Cycling

When neurons fire, release of neurotransmitter is triggered by the arrival of an action potential and occurs via exocytosis. Within the presynaptic nerve terminal, synaptic vesicles containing neurotransmitter are mobilized to the presynaptic membrane, where they are docked and primed for calcium ion sensing and, ultimately, fusion with the presynaptic membrane. The arriving action potential produces an influx of calcium ions through voltage-dependent, calcium-selective ion channels at the down stroke of the action potential (tail current). Calcium ions then trigger a biochemical cascade which results in synaptic vesicles fusing with the presynaptic membrane and releasing neurotransmitter into the synaptic cleft. Synaptic vesicle fusion is driven by the action of a set of proteins in the presynaptic terminal known as SNAREs. The membrane added by this fusion is later retrieved by endocytosis, which involves steps of clathrin-mediated nucleation, invagination of the presynaptic membrane, fission of the synaptic vesicle from the presynaptic membrane, and removal of clathrin from the endocytosed synaptic vesicles. The endocytosed synaptic vesicles are translocated and sorted within the presynaptic terminal and their lumens re-loaded with neurotransmitter.

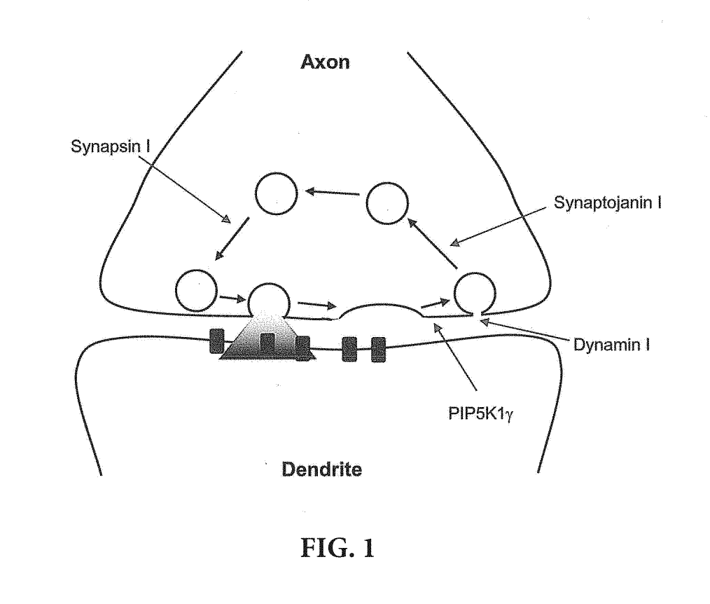

It is known that calcineurin is involved in synaptic vesicle cycling. For example, calcineurin dephosphorylates various synaptic vesicle cycling proteins that are involved in regulating synaptic vesicle cycling including, but not limited to, the synapsins and the dephosphins. The synapsins are a family of proteins that regulate synaptic vesicle mobilization and neurotransmitter release at synapses. Specifically, synapsins are thought to be involved in regulating the number of synaptic vesicles available for release via exocytosis at any given time. It is thought, for example, that dephosphorylation of synapsin I by calcineurin enhances synaptic vesicle mobilization during strong stimulation. The dephosphins regulate the endocytosis of synaptic vesicles which have released their neurotransmitter. Exemplary dephosphins include, but are not limited to, dynamin I, PIP5K1γ, synaptojanin I, Eps15, epsin, AP2, AP180. A schematic diagram of the synaptic vesicle cycle showing where certain presynaptic proteins are thought to function is shown in FIG. 1.

It has been reported that there may be a limited capacity for synaptic vesicle internalization within presynaptic terminals (as described in Balaji, J., et al., (2008). J Neurosci, 28(26):6742-6749, herein incorporated by reference). At low intensity stimulations (e.g., less than 100 action potentials triggered at less than 10 Hz), the capacity is not saturated and increasing stimulation strengths within that range does not alter the kinetics of endocytosis. At high intensity stimulations (e.g., greater than 100 action potentials triggered at greater than 10 Hz), the capacity is saturated, resulting in a decreased rate, on average, of the endocytosis of synaptic vesicles. It is further thought that the synaptic vesicle endocytosis capacity is regulated by intracellular Ca⁺⁺ concentrations within the presynaptic terminal. Increased intracellular Ca⁺⁺ levels results in the assembly of new sites of synaptic vesicle endocytosis, perhaps caused by the activation of calcineurin and the dephosphorylation of the dephosphins.

Platforms and Methods for Identifying and/or Characterizing Modulating Substances

The present invention provides systems and methods for identifying substances that modulate the synaptic vesicle cycle. Such methods typically involve cells or cell cultures comprising presynaptic terminals which exhibit functional synaptic vesicle cycling or at least one aspect of synaptic vesicle cycling, a reporter (e.g., a reporter useful for tracking synaptic vesicle cycling or at least one activity of synaptic vesicle cycling), a stimulation system (e.g., an electrical, acoustic, ultrasonic, optical or biochemical stimulation system to trigger synaptic vesicle cycling, for example, via action potential initiation), and a detection system (e.g., an imaging apparatus to capture signal generated by the reporter).

Platforms are provided for analyzing synaptic vesicle cycling (which may be referred herein as synaptic vesicle cycling platforms). Certain platforms disclosed herein are suitable for analyzing synaptic vesicle cycling in a high throughput manner. Platforms are typically configured to detect at least one aspect of synaptic vesicle cycling in each of a plurality of neuronal cell cultures. Such platforms may be configured for simultaneous induction of action potentials in each of a plurality of neuronal cell cultures and simultaneous detection of an aspect synaptic vesicle cycling in each of the cultures. Alternatively, such platforms may be configured for sequential induction of action potentials in each of a plurality of neuronal cell cultures and sequential detection of an aspect synaptic vesicle cycling in each of the cultures. In some embodiments of the invention, action potentials are induced in parallel with detection of a luminescent signal indicative of an aspect of synaptic vesicle cycling.

Platforms may include a plurality of vessels, e.g., wells, tubes, etc, that are suitable for culturing neuronal cells, and a plurality of stimulators, e.g., electrode pairs, auditory devices, etc., configured for triggering or inducing action potentials in neuronal cells in a vessel, e.g., a well. Stimulators are typically configured for placement in, or adjacent to, a vessel and to induce synaptic vesicle cycling in a plurality of neuronal cells in the vessel. For example, electrode pairs may be configured for placement in a well and to produce one or more voltage pulses that induce synaptic vesicle cycling in a plurality of neuronal cells in the well. Platforms may also comprise a detection system comprising a plurality of detectors. Each detector is typically configured to detect a luminescent signal from a neuronal cell present in a well. Luminescent signals typically emanate from a reporter molecule attached to a presynaptic protein of a neuronal cell and are indicative of an aspect of synaptic vesicle cycling in the neuronal cell. Each vessel, e.g., well, in a platform typically includes a plurality of neuronal cells.

Neuronal Cells

Systems and methods for identifying substances that modulate the synaptic vesicle cycle typically involve use of neuronal cells. In some embodiments, a neuronal cell has a presynaptic terminal, wherein the presynaptic terminal forms a functional synaptic vesicle cycle. In some embodiments, a neuronal cell does not have a presynaptic terminal, but can be induced to form a presynaptic terminal, wherein the presynaptic terminal has a functional synaptic vesicle cycle. Cells may be induced to form presynaptic terminals, wherein the presynaptic terminals have a functional synaptic vesicle cycle, by culturing and/or incubating the cells under particular conditions.

Neuronal cell cultures may comprise collections of cells which undergo one or more elements of presynaptic vesicle cycling. In some embodiments, relevant activities of the synaptic vesicle cycle include mobilization of synaptic vesicles to the presynaptic membrane, docking at the presynaptic membrane, priming for fusion with the presynaptic membrane, Ca²⁺ sensing, fusion with the presynaptic membrane, release of neurotransmitters into the synaptic cleft, retrieval of synaptic vesicles in the presynaptic terminal by endocytosis (which involves steps of clathrin-mediated nucleation, invagination of the presynaptic membrane, fission of the synaptic vesicle from the presynaptic membrane, and removal of clathrin from the endocytosed synaptic vesicles), translocation of synaptic vesicles within the presynaptic terminal, sorting of synaptic vesicles within the presynaptic terminal, and loading of synaptic vesicle lumen with neurotransmitter. In some embodiments, members of a plurality of neuronal cells are substantially similar to one another. In some embodiments, members of a plurality of neuronal cells are not substantially similar to one another.

In some embodiments, a neuronal cell is in an in vitro culture of living cells. In some embodiments, a neuronal cell system is in an in vitro suspension of cells (e.g., in a medium comprising one or more of a buffer, salt, electrolyte, detergent, etc.). In some embodiments, a neuronal cell is in an in vitro culture comprising a plurality of cells associated with (e.g., grown on, affixed to, tethered to, etc.) a surface. In some embodiments, a neuronal cell is in an in vitro culture comprising a plurality of neuronal cells that have been substantially purified from an in vivo source (e.g., harvested from an animal). In some embodiments, a neuronal cell may be derived from the cortex of a human or animal donor. In some embodiments, a neuronal cell is a stable cell line. In some embodiments, a neuronal cell is within a live animal (e.g., rodent, human, etc.).

In some embodiments, a neuronal cell culture is a primary neuronal culture. In some embodiments, primary neuronal cultures may be derived from rodent (e.g., mouse, rat, etc.), primate (e.g., human, monkey, etc.), mammalian (e.g. cat, dog, etc.), and/or invertebrate (e.g., fly, worm, etc.) primary neurons. In some embodiments, primary neuronal cultures may be derived from primary neurons from an animal's forebrain, hippocampus, striatum, cerebellum, neuromodulatory regions (e.g., locus coeruleus, ventral tegmentum, substantia nigra, raphe nucleus, basal nucleus of Meynert, septal nuclei), spinal cord, or cerebellum. In some embodiments, a suitable neuronal cell culture may include primary rat forebrain neurons.

In some embodiments, neuronal cells are stem cells. In some embodiments, stem cells are neural stem cells. In some embodiments, stem cells are embryonic stem cells. In some embodiments, stem cells are derived from rodents (e.g., mice, rats, etc.), primates (e.g., humans, monkeys, etc.), mammals (e.g., cats, dogs, etc.), and/or invertebrates (e.g., flies, worms, etc.). In some embodiments, stem cells are differentiated into neurons in vitro, and the differentiated neurons comprise presynaptic terminals which have functional synaptic vesicle cycling. See, Fico et al. Stem Cells Dev, 17(3):573-584.

In some embodiments, neuronal cells are cell lines. In some embodiments, cell lines that can be utilized include PC12 cells, cortical cell lines, and/or immortalized neural cell lines.

In some embodiments, neuronal cell cultures may comprise cells that do not form presynaptic terminals displaying functional synaptic vesicle cycles under certain culture conditions, but will form presynaptic terminals displaying functional synaptic vesicle cycles under different culture conditions. In some embodiments, neuronal cells may comprise cells that have or form presynaptic terminals displaying functional synaptic vesicle cycles under all culture conditions.

In some embodiments, synaptic vesicle cycling assays comprise cultured neuronal cells. In some embodiments, culture medium is removed from cells to be utilized in a synaptic vesicle cycling assay and is replaced with a medium (e.g., comprising one or more of a buffer, salt, electrolyte, detergent, etc.) that is suitable for electrical stimulation (e.g., a solution having a desired electrical conductivity).

In some embodiments, neuronal cells can be transformed, transfected, infected, and/or otherwise induced to take up a desired reporter for synaptic vesicle cycling. In some embodiments, cells are infected with a virus that expresses a reporter (e.g., synaptopHluorin-expressing virus). In some embodiments, cells are transformed with a vector that drives expression of a reporter. In some embodiments, cells are transgenic with an expression cassette that drives expression of a reporter. In some embodiments, expression of a reporter is inducible (e.g., by adding certain metabolites to the culture medium). In some embodiments, cells do not express a reporter themselves, but instead, a reporter is administered directly to the cells (e.g., by adding the reporter to culture medium and/or suspension medium).

Each vessel, e.g., well, tube, etc., in a platform typically comprises a plurality of neuronal cells. The plurality of neuronal cells in a vessel, e.g., well, may be in a range of 2 to 100 neuronal cells, 100 to 1000 neuronal cells, 1000 to 10,000 neuronal cells, 10,000 to 100,000 neuronal cells, 100,000 to 1,000,000 neuronal cells, or 1,000,000 to 10,000,000 neuronal cells. The plurality of neuronal cells in a vessel, e.g., well, may be about 2 neuronal cells, about 10 neuronal cells, about 100 neuronal cells, about 1000 neuronal cells, about 10,000 neuronal cells, about 100,000 neuronal cells, about 1,000,000 neuronal cells, about 10,000,000 neuronal cells, or more neuronal cells. Other appropriate neuronal cell numbers will be apparent to the skilled artisan based on the instant disclosure.

The plurality of neuronal cells may alternatively be established relative to the cell attachment area size, e.g., well bottom area. For example, the plurality of neuronal cells may be in a range of 10 to 100 neuronal cells/mm² of attachment area, 100 to 1000 neuronal cells/mm² of attachment area, 1000 to 2000 neuronal cells/mm² of attachment area, 2000 to 3000 neuronal cells/mm² of attachment area, 3000 to 4000 neuronal cells/mm² of attachment area, 4000 to 5000 neuronal cells/mm² of attachment area, 5000 to 6000 neuronal cells/mm² of attachment area, 6000 to 7000 neuronal cells/mm² of attachment area, 7000 to 8000 neuronal cells/mm² of attachment area, 8000 to 9000 neuronal cells/mm² of attachment area, or 9000 to 10000 neuronal cells/mm² of attachment area. The plurality of neuronal cells may be about 10 neuronal cells/mm² of attachment area, about 100 neuronal cells/mm² of attachment area, about 1000 neuronal cells/mm² of attachment area, about 10,000 neuronal cells/mm² of attachment area, or more neuronal cells/mm² of attachment area. Other appropriate neuronal cell numbers per attachment area will be apparent to the skilled artisan based on the instant disclosure.

The plurality of neuronal cells may comprise a single cell type or, alternatively, at least two different neuronal cell types. The neuronal cells may be primary neurons isolated from any animal disclosed herein, e.g., rat primary neurons. The neuronal cells may be selected from the group consisting of: glutamatergic, GABAergic, dopaminergic, adrenergic, serotonergic, and cholinergic neuronal cells. Still other exemplary neuronal cells are disclosed herein and are known in the art.

Reporters

Reporters, also referred herein to as reporter molecules, suitable for the present invention include any reporter proteins, genes, compounds, or other molecules or substances that are capable of generating or producing signals indicative of one or more synaptic vesicle cycling activities. For example, FM dyes (e.g., FM1-43, FM2-10, FM4-64, etc.) and fluorescently tagged antibodies against the lumenal domain of synaptic vesicle proteins (e.g., synaptotagmin antibodies) can be used to track the synaptic vesicle cycle. FM1-43 is a styrylpyridinium molecule (also known as a styryl molecule or styryl dye), which is an ampiphilic molecule having both a hydrophilic and a hydrophobic region. The hydrophilic region (typically referred to as head) containing a pyridinium group made up of two aromatic rings with a double bond bridge in between them is the fluorophore part of the dye molecule. This fluorophore group has excitation at about 500 nanometers and emission of light at about 625 nanometers.

The hydrophobic region (also referred to as tail) of the FM1-43 molecule is what allows the dye to get into the plasma membrane of a cell or a synaptic vesicle. The interaction of the hydrocarbon tail may also cause the change in brightness of the signal. However, these reporters typically require a large amount of washing to reduce the background. Thus, these reporters may not be desirable for homogenous cell-based assays. As used herein, “homogenous cell-based assays” refer to assays that use a reporter that does not require washing following the application of the reporter to the cells. In addition, due to the need for washing, these reporters typically cannot track the kinetic process of endocytosis because endocytosis generally occurs prior to and during the washes. Washing steps also make high throughput screening more difficult, costly, and time-consuming.

In some embodiments, reporters that can be utilized in synaptic vesicle cycling assays are pH-sensitive reporters. Typically, such reporters may have certain characteristic(s) at certain pH or pH ranges, and other characteristic(s) at different pH or pH ranges. For example, pH-sensitive reporters may have different fluorescent characteristics in extracellular environment (about pH 7.4) and the synaptic vesicle lumen (about pH 5.5). For example, pH-sensitive fluorescent reporters suitable for the invention may fluoresce at about pH 7.4, but not at pH 5.5. pH-sensitive fluorescent reporters suitable for the invention may also fluoresce at about pH 5.5, but not at pH 7.4. pH-sensitive reporters that function in this way are useful because non-endocytosed reporter does not need to be washed away.

Thus, pH-sensitive reporters typically remove the need for the washing steps, making the assay homogenous and allowing for the direct measurement of endocytosis. Examples of such reporters include: 1) pHluorin tagged to the lumenal domain of synaptic vesicle proteins; and 2) pH sensitive dyes (e.g., pHrodo and CypHer5E) tagged to the lumenal domain of synaptic vesicle proteins or formulated to intercalate the synaptic vesicle membrane (similar to FM1-43).

pHluorin is a pH-sensitive green fluorescent protein (GFP). It can be used to track the synaptic vesicle cycle by tagging to it the lumenal domain of synaptic vesicle proteins. The lumenal domain tag helps to target pHluorin to the inside of synaptic vesicles. pHluorin is fluorescent at ˜pH 7.4; fluorescence is quenched at ˜pH 5.5. pHluorin can be tagged to VAMP2, vGlut1, synaptophysin, and synaptotagmin (see, Miesenbock, et al. (1998) Nature, 394(6689):192-5; Fernandez-Alfonso et al. (2006) Neuron, 51(2):179-86; Granseth et al. (2006) Neuron, 51(6):773-86; Voglmaier et al. (2006) Neuron, 51(1):71-86). pHluorin can be delivered to neurons (e.g., in a multiwell plate) by a variety of methods, including infection with a virus expressing pHluorin tagged synaptic vesicle proteins, transformation with a vector that drives expression of pHluorin tagged synaptic vesicle proteins, or generation of a transgenic animal (e.g., mouse or rat) expressing pHluorin tagged synaptic vesicle proteins.

A synaptic vesicle protein is a presynaptic protein that associates with a synaptic vesicle. A synaptic vesicle protein typically has a transmembrane portion and may have a lumenal portion and/or a cytoplasmic portion. The synaptic vesicle protein may be selected from the group consisting of: VAMP2, vGlut1, synaptophysin, vesicular GABA transporter; acetylcholine transporter, catecholamine transporter or synaptotagmin. Typically, the synaptic vesicle protein is attached to a reporter molecule. For example, the reporter molecule may be attached to the lumenal portion of the synaptic vesicle protein. Typically, the reporter molecule is a pH sensitive reporter molecule. For example, the reporter molecule may be a pH sensitive fluorescent protein, e.g., a pHluorin.

A pH sensitive reporter molecule, e.g., a pH sensitive fluorescent protein, e.g., a pHluorin, may have a peak emission intensity at a pH in a range of 4.5 to 5.0, 5.0 to 5.5, 6.5 to 7.0, 7.0 to 7.5, 7.5 to 8.0 or 8.0 to 8.5. The pH sensitive reporter molecule may have a maximal emission intensity at a pH of up to about 4.5, 4.5, 5.0, 5.5, 6.0, 6.5, 7.0, 7.5, 8.0, 8.5 or more, including all values between the numbers of the series. The pH sensitive reporter molecule may fluoresce at a pH in a range of 6.5 to 8.5, with an intensity that is significantly greater, e.g., 2-fold higher, 5-fold higher, 10-fold higher, 20-fold higher, 30-fold higher, 40-fold higher, or more, than at a pH in a range of 4.5 to 6.5. The pH sensitive reporter molecule may fluoresce at a pH in a range of 6.5 to 7.5, with an intensity that is significantly greater, e.g., 2-fold higher, 5-fold higher, 10-fold higher, 20-fold higher, 30-fold higher, 40-fold higher, or more, than at a pH in a range of 5.0 to 6.0. Alternatively, the pH sensitive reporter molecule may fluoresce at a pH in a range of 4.5 to 6.5, with an intensity that is significantly greater, e.g., 2-fold higher, 5-fold higher, 10-fold higher, 20-fold higher, 30-fold higher, 40-fold higher, or more, than at a pH in a range of 6.5 to 8.5. The pH sensitive reporter molecule may fluoresce at a pH in a range of 5.5 to 6.0, with an intensity that is significantly greater, e.g., 2-fold higher, 5-fold higher, 10-fold higher, 20-fold higher, 30-fold higher, 40-fold higher, or more, than at a pH in a range of 6.5 to 7.5. Other appropriate pH values for the peak emission intensity of pH sensitive reporter molecules will be apparent to the skilled artisan in view of the instant disclosure.

The pH sensitive reporter molecule may have a peak emission wavelength in a range of 350 nm to 400 nm, 400 nm to 450 nm, 450 nm to 500 nm, 500 nm to 550 nm, 550 nm to 600 nm, or 600 nm to 650 nm. The pH sensitive reporter may have a peak emission wavelength of about 300 nm, about 350 nm, about 400 nm, about 450 nm, about 500 nm, about 550 nm, about 600 nm, about 650 nm, or more. Other appropriate the peak emission wavelengths of pH sensitive reporter molecules will be apparent to the skilled artisan in view of the instant disclosure.

In some embodiments, the pHluorin, which may be used as a reporter molecule in the platforms and methods disclosed herein, has an amino acid sequence of MSKGEELFTGVVPILVELDGDVNGHKFSVSGEGEGDATYGKLTLKFICTTGKLPVPWP TLVTTLTYGVQCFSRYPDHMKRHDFFKSAMPEGYVQERTIFFKDDGNYKTRAEVKFE GDTLVNRIELKGIDFKEDGNILGHKLEYNYNDHQVYIMADKQKNGIKANFKIRHNIED GGVQLADHYQQNTPIGDGPVLLPDNHYLFTTSTLSKDPNEKRDHMVLLEFVTAAGIT HGMDELYK (SEQ ID NO: 1; gi|45479290|gb|AAS66682.1| superecliptic pHluorin). Other appropriate pHluorins will be apparent to the skilled artisan in view of the instant disclosure.

In some embodiments, the pHluorin is fused to synaptophysin. An exemplary synaptophysin based pHluorin, which may be used in the platforms and methods disclosed herein, is encoded by the following nucleic acid sequence:

(SEQ ID NO: 2; synaptophysin-pHluorin)
ATGGACGTGGTGAATCAGCTGGTGGCTGGGGGTCAGTTCCGGGTGGTCAA
GGAGCCCCTTGGCTTCGTGAAGGTGCTGCAGTGGGTCTTTGCCATCTTCG
CCTTTGCTACGTGTGGCAGCTACACCGGGGAGCTTCGGCTGAGCGTGGAG
TGTGCCAACAAGACGGAGAGTGCCCTCAACATCGAAGTTGAATTCGAGTA
CCCCTTCAGGCTGCACCAAGTGTACTTTGATGCACCCTCCTGCGTCAAAG
GGGGCACTACCAAGATCTTCCTGGTTGGGGACTACTCCTCGTCGGCTGAA
TTCTTTGTCACCGTGGCTGTGTTTGCCTTCCTCTACTCCATGGGGGCCCT
GGCCACCTACATCTTCCTGCAGAACAAGTACCGAGAGAACAACAAAGGGC
CTATGATGGACTTTCTGGCTACAGCCGTGTTCGCTTTCATGTGGCTAGTT
AGTTCATCAGCCTGGGCCAAAGGCCTGTCCGATGTGAAGATGGCCACGGA
CCCAGAGAACATTATCAAGGAGATGCCCATGTGCCGCCAGACAGGGAACA
CCGGTATGAGTAAAGGAGAAGAACTTTTCACTGGAGTTGTCCCAATTCTT
GTTGAATTAGATGGTGATGTTAATGGGCACAAATTTTCTGTCAGTGGAGA
GGGTGAAGGTGATGCAACATACGGAAAACTTACCCTTAAATTTATTTGCA
CTACTGGAAAACTACCTGTTCCATGGCCAACACTTGTCACTACTTTAACT
TATGGTGTTCAATGCTTTTCAAGATACCCAGATCATATGAAACGGCATGA
CTTTTTCAAGAGTGCCATGCCCGAAGGTTATGTACAGGAAAGAACTATAT
TTTTCAAAGATGACGGGAACTACAAGACACGTGCTGAAGTCAAGTTTGAA
GGTGATACCCTTGTTAATAGAATCGAGTTAAAAGGTATTGATTTTAAAGA
AGATGGAAACATTCTTGGACACAAATTGGAATACAACTATAACGATCACC
AGGTGTACATCATGGCAGACAAACAAAAGAATGGAATCAAAGCTAACTTC
AAAATTAGACACAACATTGAAGATGGAGGCGTTCAACTAGCAGACCATTA
TCAACAAAATACTCCAATTGGCGATGGGCCCGTCCTTTTACCAGACAACC
ATTACCTGTTTACAACTTCTACTCTTTCGAAAGATCCCAACGAAAAGAGA
GACCACATGGTCCTTCTTGAGTTTGTAACAGCTGCTGGGATTACACATGG
CATGGATGAACTATACAAAACCGGTCGCCAGACAGGGAACACATGCAAGG
AACTGAGGGACCCTGTGACTTCAGGACTCAACACCTCAGTGGTGTTTGGC
TTCCTGAACCTGGTGCTCTGGGTTGGCAACTTATGGTTCGTGTTCAAGGA
GACAGGCTGGGCAGCCCCATTCATGCGCGCACCTCCAGGCGCCCCGGAAA
AGCAACCAGCACCTGGCGATGCCTACGGCGATGCGGGCTACGGGCAGGGC
CCCGGAGGCTATGGGCCCCAAGACTCCTACGGGCCTCAGGGTGGTTATCA
ACCCGATTACGGGCAGCCAGCCAGCGGTGGCGGTGGCTACGGGCCTCAGG
GCGACTATGGGCAGCAAGGCTATGGCCAACAGGGTGCGCCCACCTCCTTC
TCCAATCAGATGTAA.

In some embodiments, the pHluorin is fused to VAMP2. In other embodiments, the pHluorin is fused to vGlut. An exemplary vGlut based pHluorin, which may be used in the platforms and methods disclosed herein, is encoded by the following nucleic acid sequence:

(SEQ ID NO: 3; vGlut-pHluorin)
ATGGAGTTCAGACAGGAGGAGTTCCGAAAACTTGCAGGCCGAGCACTGGG
TCGACTGCATCGCTTGCTGGAGAAGCGGCAGGAGGGAGCAGAAACACTGG
AGCTCAGCGCTGACGGCCGGCCCGTCACAACCCACACTAGAGACCCACCT
GTGGTAGACTGTACCTGCTTTGGCCTGCCTAGAAGGTATATCATTGCAAT
CATGTCTGGGCTTGGCTTCTGCATCAGTTTTGGAATTAGATGTAACCTGG
GGGTTGCGATTGTGAGTATGGTCAATAACTCAACGACACACAGAGGAAGC
ACCTCCGGTGGATCCGGGGGGACTGGGGGAAGCATGTCCAAAGGCGAAGA
ACTGTTTACAGGCGTGGTGCCAATACTCGTGGAACTCGACGGCGATGTTA
ACGGACATAAGTTTTCCGTGTCCGGCGAGGGTGAGGGAGATGCTACATAT
GGAAAACTGACTCTGAAATTTATTTGTACCACCGGGAAACTGCCCGTGCC
ATGGCCTACCCTCGTCACTACACTGACGTATGGGGTTCAATGTTTCAGTA
GGTATCCGGACCACATGAAAAGACACGATTTCTTCAAAAGCGCAATGCCC
GAAGGCTACGTACAAGAACGCACTATATTCTTCAAGGACGACGGCAACTA
TAAAACCCGCGCAGAGGTTAAATTTGAGGGGGACACACTGGTAAATCGGA
TCGAGCTGAAGGGTATCGACTTTAAAGAAGACGGAAACATATTGGGCCAT
AAGCTGGAATACAACTATAATGATCATCAGGTGTATATCATGGCCGATAA
GCAGAAAAATGGAATCAAGGCAAACTTCAAAATTAGGCACAATATTGAGG
ATGGGGGCGTCCAGCTGGCCGACCATTACCAGCAGAATACTCCAATAGGC
GATGGACCGGTGTTGCTTCCTGATAATCACTACTTGTTTACCACTAGCAC
CCTCAGTAAGGATCCCAATGAGAAGCGGGACCACATGGTCCTGCTGGAGT
TCGTGACTGCCGCTGGCATAACTCATGGGATGGATGAGCTCTACAAAGGA
GGGACAGGGGGTACCGGTGGTAGTGGTGGAACCGGCGGCCATGTAGTGGT
GCAGAAGGCCCAGTTCAATTGGGACCCAGAGACAGTGGGCTTGATCCACG
GCTCTTTCTTTTGGGGTTATATCGTTACTCAAATCCCAGGCGGTTTCATT
TGTCAGAAGTTCGCCGCCAACAGAGTGTTCGGCTTCGCAATAGTAGCCAC
CTCCACACTGAACATGCTGATTCCGTCTGCAGCCCGAGTGCACTATGGCT
GTGTGATATTCGTGAGAATCCTGCAAGGTCTTGTCGAGGGCGTGACCTAC
CCAGCCTGTCACGGGATCTGGTCCAAATGGGCTCCACCACTGGAGCGATC
CAGACTGGCGACCACTGCATTCTGTGGCAGTTACGCCGGCGCTGTCGTGG
CTATGCCCCTGGCTGGTGTGCTGGTGCAGTACAGCGGATGGAGCAGCGTG
TTCTACGTTTATGGCTCTTTCGGGATCTTTTGGTACCTCTTCTGGCTGCT
CGTCAGCTACGAATCCCCCGCTCTGCACCCTTCCATCAGCGAGGAGGAGC
GGAAATACATTGAAGACGCAATCGGTGAATCAGCAAAGCTGATGAACCCC
GTGACGAAATTCAATACACCGTGGAGACGGTTTTTTACCTCCATGCCCGT
GTACGCGATAATTGTTGCAAATTTCTGCCGGAGCTGGACATTCTATCTCC
TCCTGATTAGCCAACCTGCCTACTTCGAAGAGGTCTTCGGGTTCGAGATC
TCTAAAGTGGGTCTTGTGTCTGCGCTCCCTCACCTCGTTATGACCATTAT
TGTTCCTATCGGCGGGCAGATCGCTGATTTTCTTCGGTCAAGACATATTA
TGTCCACTACTAATGTGAGGAAGCTGATGAATTGCGGTGGGTTTGGCATG
GAGGCGACCCTGCTTCTGGTGGTGGGTTACTCTCATTCCAAGGGAGTGGC
GATCAGTTTCCTGGTCCTGGCCGTGGGATTTAGCGGCTTTGCCATAAGCG
GTTTTAACGTTAATCACCTTGATATTGCTCCCCGCTACGCATCCATTCTC
ATGGGGATCAGCAACGGAGTCGGGACTCTGAGTGGAATGGTTTGCCCAAT
AATAGTCGGAGCGATGACTAAACATAAGACCCGAGAAGAATGGCAGTACG
TGTTCCTGATTGCTAGTCTGGTCCATTACGGAGGCGTCATCTTCTATGGC
GTCTTCGCCTCTGGGGAAAAACAACCATGGGCAGAGCCTGAAGAAATGTC
CGAGGAAAAATGTGGGTTCGTCGGGCACGATCAGCTTGCCGGTTCTGATG
AGTCCGAGATGGAGGACGAGGTTGAGCCCCCTGGGGCGCCTCCTGCACCG
CCCCCAAGCTACGGCGCAACCCATAGCACTGTGCAGCCTCCTCGGCCACC
TCCTCCAGTGCGGGATTACTAA.

The sequence of a human synapsin I promoter, which may be used to control expression of a reporter molecule, e.g., a pHluorin, in the platforms and methods disclosed herein, is provided as follows. ACTAATCTCCCCGCGGGCACTGCGTGTGACCTCACCCCCCTCTGTGAGGGGGTTAT TTCTCTACTTTCGTGTCTCTGAGTGTGCTTCCAGTGCCCCCCTCCCCCCAAAAAATG CCTTCTGAGTTGAATATCAACACTACAAACCGAGTATCTGCAGAGGGCCCTGCGTA TGAGTGCAAGTGGGTTTTAGGACCAGGATGAGGCGGGGTGGGGGTGCCTACCTGA CGACCGACCCCGACCCACTGGACAAGCACCCAACCCCCATTCCCCAAATTGCGCA TCCCCTATCAGAGAGGGGGAGGGGAAACAGGATGCGGCGAGGCGCGTGCGCACT GCCAGCTTCAGCACCGCGGACAGTGCCTTCGCCCCCGCCTGGCGGCGCGCGCCAC CGCCGCCTCAGCACTGAAGGCGCGCTGACGTCACTCGCCGGTCCCCCGCAAACTC CCCTTCCCGGCCACCTTGGTCGCGTCCGCGCCGCCGCCGGCCCAGCCGGACCGCAC CACGCGAGGCGCGAGATAGGGGGGCACGGGCGCGACCATCTGCGCTGCGGCGCC GGCGACTCAGCGCTGCCTCAGTCTGCGGTGGGCAGCGGAGGAGTCGTGTCGTGCC TGAGAGCGCAG (SEQ ID NO: 4; human synapsin I promoter). Other appropriate promoters may be used. For example, for general expression CMV or chicken beta-actin promoters may be used. Alternatively, for neuron-specific expression neuron specific enolase or synapsin I promoters may be used. For glutamatergic-specific expression vGlut1 promoter may be used. For GABAergic-specific expression GAD65, GAD67, somatostatin, or GABA vesicular transporter promoters may be used. Still other appropriate promoters will be apparent to the skilled artisan.

The following is an exemplary expression cassette having synaptophysin-pHluorin operably linked to a human synapsin I promoter, which may be used in the platforms and methods disclosed herein.

(SEQ ID NO: 5; hSyn-SypHy Cassette Sequence)
ACTAATCTCCCCGCGGGCACTGCGTGTGACCTCACCCCCCTCTGTGAGGG
GGTTATTTCTCTACTTTCGTGTCTCTGAGTGTGCTTCCAGTGCCCCCCTC
CCCCCAAAAAATGCCTTCTGAGTTGAATATCAACACTACAAACCGAGTAT
CTGCAGAGGGCCCTGCGTATGAGTGCAAGTGGGTTTTAGGACCAGGATGA
GGCGGGGTGGGGGTGCCTACCTGACGACCGACCCCGACCCACTGGACAAG
CACCCAACCCCCATTCCCCAAATTGCGCATCCCCTATCAGAGAGGGGGAG
GGGAAACAGGATGCGGCGAGGCGCGTGCGCACTGCCAGCTTCAGCACCGC
GGACAGTGCCTTCGCCCCCGCCTGGCGGCGCGCGCCACCGCCGCCTCAGC
ACTGAAGGCGCGCTGACGTCACTCGCCGGTCCCCCGCAAACTCCCCTTCC
CGGCCACCTTGGTCGCGTCCGCGCCGCCGCCGGCCCAGCCGGACCGCACC
ACGCGAGGCGCGAGATAGGGGGGCACGGGCGCGACCATCTGCGCTGCGGC
GCCGGCGACTCAGCGCTGCCTCAGTCTGCGGTGGGCAGCGGAGGAGTCGT
GTCGTGCCTGAGAGCGCAGTCGAGAATTCAAGCTGCTAGCAAGGATCCAC
CGGTCGCCACCATGGACGTGGTGAATCAGCTGGTGGCTGGGGGTCAGTTC
CGGGTGGTCAAGGAGCCCCTTGGCTTCGTGAAGGTGCTGCAGTGGGTCTT
TGCCATCTTCGCCTTTGCTACGTGTGGCAGCTACACCGGGGAGCTTCGGC
TGAGCGTGGAGTGTGCCAACAAGACGGAGAGTGCCCTCAACATCGAAGTT
GAATTCGAGTACCCCTTCAGGCTGCACCAAGTGTACTTTGATGCACCCTC
CTGCGTCAAAGGGGGCACTACCAAGATCTTCCTGGTTGGGGACTACTCCT
CGTCGGCTGAATTCTTTGTCACCGTGGCTGTGTTTGCCTTCCTCTACTCC
ATGGGGGCCCTGGCCACCTACATCTTCCTGCAGAACAAGTACCGAGAGAA
CAACAAAGGGCCTATGATGGACTTTCTGGCTACAGCCGTGTTCGCTTTCA
TGTGGCTAGTTAGTTCATCAGCCTGGGCCAAAGGCCTGTCCGATGTGAAG
ATGGCCACGGACCCAGAGAACATTATCAAGGAGATGCCCATGTGCCGCCA
GACAGGGAACACCGGTATGAGTAAAGGAGAAGAACTTTTCACTGGAGTTG
TCCCAATTCTTGTTGAATTAGATGGTGATGTTAATGGGCACAAATTTTCT
GTCAGTGGAGAGGGTGAAGGTGATGCAACATACGGAAAACTTACCCTTAA
ATTTATTTGCACTACTGGAAAACTACCTGTTCCATGGCCAACACTTGTCA
CTACTTTAACTTATGGTGTTCAATGCTTTTCAAGATACCCAGATCATATG
AAACGGCATGACTTTTTCAAGAGTGCCATGCCCGAAGGTTATGTACAGGA
AAGAACTATATTTTTCAAAGATGACGGGAACTACAAGACACGTGCTGAAG
TCAAGTTTGAAGGTGATACCCTTGTTAATAGAATCGAGTTAAAAGGTATT
GATTTTAAAGAAGATGGAAACATTCTTGGACACAAATTGGAATACAACTA
TAACGATCACCAGGTGTACATCATGGCAGACAAACAAAAGAATGGAATCA
AAGCTAACTTCAAAATTAGACACAACATTGAAGATGGAGGCGTTCAACTA
GCAGACCATTATCAACAAAATACTCCAATTGGCGATGGGCCCGTCCTTTT
ACCAGACAACCATTACCTGTTTACAACTTCTACTCTTTCGAAAGATCCCA
ACGAAAAGAGAGACCACATGGTCCTTCTTGAGTTTGTAACAGCTGCTGGG
ATTACACATGGCATGGATGAACTATACAAAACCGGTCGCCAGACAGGGAA
CACATGCAAGGAACTGAGGGACCCTGTGACTTCAGGACTCAACACCTCAG
TGGTGTTTGGCTTCCTGAACCTGGTGCTCTGGGTTGGCAACTTATGGTTC
GTGTTCAAGGAGACAGGCTGGGCAGCCCCATTCATGCGCGCACCTCCAGG
CGCCCCGGAAAAGCAACCAGCACCTGGCGATGCCTACGGCGATGCGGGCT
ACGGGCAGGGCCCCGGAGGCTATGGGCCCCAAGACTCCTACGGGCCTCAG
GGTGGTTATCAACCCGATTACGGGCAGCCAGCCAGCGGTGGCGGTGGCTA
CGGGCCTCAGGGCGACTATGGGCAGCAAGGCTATGGCCAACAGGGTGCGC
CCACCTCCTTCTCCAATCAGATGTAA.

Exemplary pH-sensitive dyes, such as, Cypher5E (GE Healthcare) and pHrodo (Invitrogen), are small molecule fluorophores which fluoresce at ˜pH 5.5 and are quenched at ˜pH 7.4. For example, the differential fluorescence of Cypher5E at different pH occurs because it has a different structure at pH 5.5 than it does at pH 7.4 (FIG. 12). In some embodiments, pH-sensitive dyes can be used to track vesicle exocytosis, endocytosis, and/or reacidification. Typically, pH-sensitive dyes are cell membrane permeable.

Typically, pH-sensitive dyes may be associated with one or more targeting moieties that direct transport of the pH-sensitive dye into synaptic vesicles and prevent diffusion out of synaptic vesicles. In some embodiments, the association is covalent. In some embodiments, the association is non-covalent (e.g., mediated by hydrogen bonding, electrostatic interactions, affinity interactions, Van der Waals forces, etc.). To give but a few examples, pH-sensitive dyes can be associated with a membrane intercalater (similar to FM1-43); an antibody that recognizes intralumenal domain(s) of synaptic vesicle protein(s); an antibody that recognizes a protein tag that is present in synaptic vesicle lumens at levels higher than it is present outside of synaptic vesicle lumens. pH-sensitive dyes can also be bound to large, membrane impermeable non-specific molecules such as dextran. They get into synaptic vesicles by diffusion when the vesicle is on the surface of the cell.

Electrical Stimulation System

Assays for identifying and/or characterizing modulating substances in accordance with the present invention typically involve an electrical stimulation system. Field stimulation of action potential firing is based on the physical interaction of an external electric field with relatively simple resistive properties of the cell membranes of cells. If one thinks of a typical cell body as an object with a high resistance shell (the plasma membrane) and a relatively low resistance interior (the cytoplasm), simple electrostatic theory implies that, if placed in an electric field, it will have a voltage drop across the cell given by the product of the electric field intensity E and the size of the cell body d. For example, one can think of the voltage drop as occurring across a series of resistors, two corresponding to the plasma membrane and one for the cytoplasm. Given that the plasma membrane has a much higher resistance than the cytoplasm, this implies that virtually all of the voltage drop will occur across the plasma membrane. On one side of the cell the membrane will depolarize by ˜(E×d)/2, while on the opposite side it will hyperpolarize by the same amount. For neurons, the expectation is that if one side depolarizes enough it will initiate an action potential. Potential stimulation typically involves a pair of electrodes with a voltage drop between them (e.g., ˜10 V/cm) such that an electric field is generated. As a non-limiting example, for a typical cell body of diameter 50 μm, the voltage drop across each cell membrane is about 25 mV.

In various embodiments, an electrical stimulation system is used to excite action potentials in neuronal cells. In certain embodiments, an electrical stimulation system provides cellular transmembrane potentials sufficient to induce action potentials in neuronal cells. In some embodiments, an electrical stimulation system may be adapted to excite action potentials substantially simultaneously in a plurality of separate wells comprising neuronal cells, e.g., in plural wells of multi-well plates. An electrical stimulation system may be adapted to provide a variety of, or customized, stimulation waveforms to one or more wells containing viable neuronal cells. Stimulation waveforms may comprise time-varying electric fields.

Electrodes of an electrode pair may have a substantially curvilinear surface. For example, the electrodes of an electrode pair may be shaped as concentric cylinders. The electrodes of an electrode pair, which have different diameters, may be arranged as concentric cylinders with the smaller diameter electrode being positioned substantially within the lumenal portion of the larger diameter electrode; optionally, the two concentric cylindrical electrodes may be separated by an annular insulating material. The platforms disclosed herein may comprise a positioning device, e.g., an electrode transfer system configured to operably position each electrode pair of the plurality of electrode pairs into one well of the plurality of wells. For example, commercially available multiwell plate automation equipment may be adapted for use as a an electrode transfer system, e.g., Biomek® laboratory automation equipment.

Platforms comprising a electrical stimulation system may comprise a power source operably linked to the plurality of electrode pairs. The power source is typically configured to apply a predetermined voltage, e.g., a voltage pulse, across each electrode pair. The voltage may be in a range of 1 V to 400 V, 1 V to 300 V, 1 V to 200 V, 1 V to 100 V, 5 V to 100 V, 5 V to 50 V, 5 V to 20 V, or 5 V to 10 V. The voltage pulse may be up to 1 V, about 2 V, about 3 V, about 4 V, about 5 V, about 6 V, about 7 V, about 8 V, about 9 V, about 10 V, about 20 V, about 30 V, about 40 V, about 50 V, about 60 V, about 70 V, about 80 V, about 90 V, about 100 V, about 200 V, about 300 V, about 400 V, or more. Other appropriate voltages will be apparent to the skilled artisan based on the instant disclosure.

Platforms may also comprise a pulse generator operably linked to the power source and the plurality of electrode pairs. The pulse generator is typically configured to apply a predetermined voltage pulse across each electrode pair. The pulse generator may be configured to apply a plurality of predetermined voltage pulses at a predetermined frequency for a predetermined time. Accordingly, the pulse generator may be configured to apply a particular voltage waveform. Exemplary waveforms are disclosed herein. A computer may be operably linked to the pulse generator, and configured to control various aspects of the voltage pulse, e.g., magnitude of each pulse, waveform associated with a plurality of pulses, duration of a pulse, frequency of pulses, duration between initiation of each pulse, etc.

The predetermined frequency produced by a pulse generator may be in a range of 0.2 Hz to 200 Hz, 0.2 Hz to 100 Hz, 0.2 Hz to 50 Hz, 0.2 Hz to 40 Hz, 0.2 Hz to 30 Hz, 0.2 Hz to 20 Hz, 0.2 Hz to 10 Hz, 10 Hz to 20 Hz, 10 Hz to 30 Hz, 10 Hz to 40 Hz, 10 Hz to 50 Hz, or 10 to 100 Hz. The predetermined frequency may be up to 0.2 Hz, about 1 Hz, about 2 Hz, about 3 Hz, about 4 Hz, about 4 Hz, about 5 Hz, about 10 Hz, about 15 Hz, about 20 Hz, about 30 Hz, about 40 Hz, about 50 Hz, about 60 Hz, about 70 Hz, about 80 Hz, about 90 Hz, about 100 Hz, or more. Other appropriate frequencies will be apparent to the skilled artisan based on the instant disclosure.

The predetermined time that a pulse generator produces a voltage, or plurality of voltages, may be in a range of 0.1 second to 2 minutes, in a range of 0.1 second to 1 minute, 0.1 second to 45 seconds, 0.1 second to 30 seconds, 0.1 second to 20 seconds, 0.1 second, to 15 seconds, 0.1 second to 10 seconds, 0.1 second to 5 seconds, or 0.1 second to 1 second. The predetermined time may be about 0.1 second, about 0.5 second, about 1 second, about 2 seconds, about 3 seconds, about 4 seconds, about 5 seconds, about 10 seconds, about 20 seconds, about 30 seconds, about 40 seconds, about 50 seconds, about 1 minute, about 2 minutes, or more. Other appropriate times will be apparent to the skilled artisan based on the instant disclosure.

The duration of a pulse produced by a voltage generator may vary. For example, each pulse in a plurality of pulse may be substantially equal in duration or may have a different duration from other pulses in the plurality. The duration of a pulse may be in a range of 0.1 millisecond (msec) to 10 msec, 0.1 msec to 5 msec, 0.1 msec to 4 msec, 0.1 msec to 3 msec, 0.1 msec to 2 msec, 0.1 msec to 1 msec, or 0.1 msec to 0.5 msec. The duration of a pulse may be about 0.1 msec, about 0.2 msec, about 0.3 msec, about 0.4 msec, about 0.5 msec, about 0.6 msec, about 0.7 msec, about 0.8 msec, about 0.9 msec, about 1 msec, about 2 msec, about 3 msec, about 4 msec, about 5 msec, about 10 msec, about 20 msec, or more. Other appropriate durations will be apparent to the skilled artisan based on the teachings of the instant disclosure.

The number of pulses, e.g., in a plurality of pulses, produced by a pulse generator may also vary. The number of pulses may be in a range of 1 to 5000, 1 to 2000, 1 to 1000, 1 to 500, 1 to 400, 1 to 300, 1 to 200, 1 to 100, 1 to 50, 1 to 40, 1 to 30, 1 to 20, 1 to 10, or 1 to 5. The number of pulses may be about 1, about 2, about 3, about 4, about 5, about 10, about 20, about 30, about 40, about 50, about 100, about 200, about 300, about 400, about 500, about 1000, about 2000, about 3000, about 4000, about 5000, or more. Other appropriate pulse numbers will be apparent to the skilled artisan based on the teachings of the instant disclosure.

The duration between initiation of each pulse in a plurality of pulses may vary. The duration between initiation of each pulse in a plurality of pulses may be in a range of 0.1 msec to 20 msec, 0.1 msec to 10 msec, 0.1 msec to 5 msec, 0.1 msec to 4 msec, 0.1 msec to 3 msec, 0.1 msec to 2 msec, 0.1 msec to 1 msec, or 0.1 msec to 0.5 msec. The duration between initiation of each pulse in a plurality of pulses may be about 0.1 msec, about 0.2 msec, about 0.3 msec, about 0.4 msec, about 0.5 msec, about 0.6 msec, about 0.7 msec, about 0.8 msec, about 0.9 msec, about 1 msec, about 2 msec, about 3 msec, about 4 msec, about 5 msec, about 6 msec, about 7 msec, about 8 msec, about 9 msec, about 10 msec, about 20 msec, or more. Other appropriate durations will be apparent to the skilled artisan based on the teachings of the instant disclosure.

In various embodiments, application of stimulation waveforms to neurons induces exocytosis and endocytosis of synaptic vesicles so that synaptic vesicle cycling may be studied under controlled laboratory conditions. In certain embodiments, a stimulation waveform is created by applying voltage or current waveforms to an electrode pair located in contact with, or in close proximity to, a neuronal cell. Application of voltage or current waveforms to electrodes can create time-varying electric fields and/or time-varying current densities within the neuronal cell, and these electric fields and/or current densities can alter cellular transmembrane potential and induce action potentials in neuronal cells.

Neurons in culture, suspension, and/or other preparation can be stimulated by application of excitation waveforms, as described in Ryan and Smith (1995, Neuron, 14:983; incorporated herein by reference) and in Ryan et al. (1996, Proc. Natl. Acad. Sci., 93:5567; incorporated herein by reference). An important factor is to maximize the contact area of the electrode with the bath containing neurons (and lower the junctional resistance) while increasing the resistance of the bath surrounding the cells. The resistance of a slab of saline solution of dimensions l (length) and cross-sectional area A is given by: R=ι×ρ/A where ρ is the conductivity of saline (˜60Ω cm for mammalian saline). For a single well in a multi-well plate, t will be roughly the diameter of the well (e.g., ˜5 mm for a 96 well plate). The value of A can be decreased by minimizing the depth the solution. For example, the wells can be designed to minimize the depth (and hence increase R). In some embodiments, a total thickness of ˜0.5 mm can be used. In some embodiments, one can build an insert that fits in the well such that two electrode sheets sit on either side of the well but allowing desirable depth of solution (e.g., ˜0.5 mm) between them.

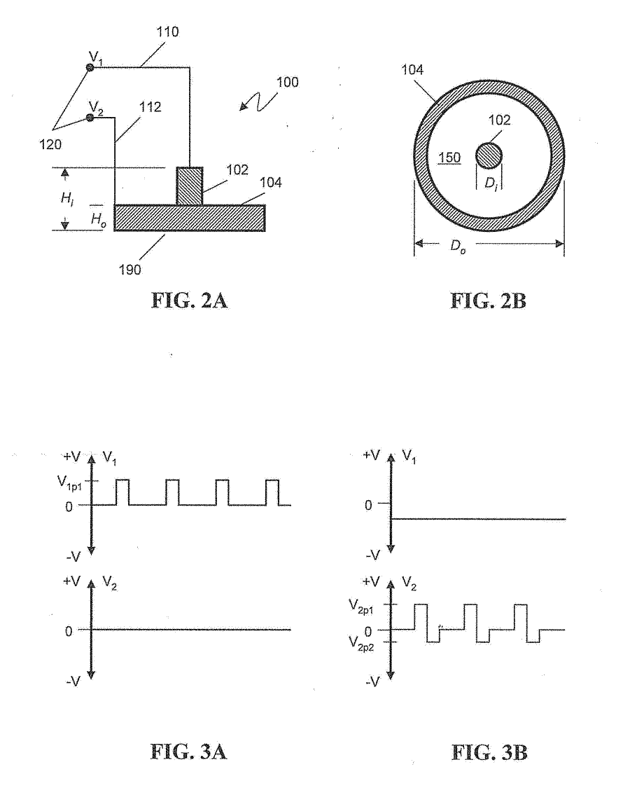

One particular example of an electrode pair 100 useful for the electrical stimulation system is shown in FIGS. 2A-B. In the example shown, the electrode assembly comprises two electrodes, an inner electrode 102 and an outer electrode 104. In various embodiments, the electrodes 102, 104 are electrically conductive. Electrodes may be made from metals, semi-metals, semi-conductors, conductive polymers, non-conductive polymers, and/or any combination thereof. When formed from non-conductive material, electrodes may be coated with conductive material. Electrodes may be formed from, or plated with, biocompatible metals or conductive materials, e.g., gold, silver, various alloys of stainless steel, titanium, platinum, conductive polymers, indium tin oxide (ITO), combinations thereof, etc. The inner electrode 102 can be a solid rod-like element, a wire, or may be cylindrically shaped with a hollow core. The inner electrode may be characterized by a height Hᵢ and a diameter Dᵢ, as indicated in FIGS. 2A-B. In various embodiments, the outer electrode 104 is cylindrical or annular in shape at least at a distal end 190 which comes into contact with, or within close proximity to, a neuronal cell culture. The outer electrode may be a ring of conductive wire, a circular band formed from a thin strip of conductive material, or a cylindrically-shaped conductive tube. The outer electrode 104 may be characterized by a height Hₒ and diameter Dₒ, as indicated in FIGS. 2A-B. In some embodiments, Hᵢ≈Hₒ and Dᵢ<Dₒ. In some embodiments, Hᵢ<Hₒ and Dᵢ<Dₒ. In some embodiments, Hᵢ>Hₒ and Dᵢ<Dₒ. In some embodiments, the inner electrode 102 acts as a cathode and the outer electrode 104 as an anode. In some embodiments, the inner electrode 102 acts as an anode and the outer electrode 104 functions as a cathode. In various embodiments, an excitation region 150 exists between the inner 102 and outer 104 electrodes.

Wires 110, 112 or their electrically-conducting equivalents, e.g., conductive traces on a circuit board, binary coaxial cable, etc., may be connected to the inner 102 and outer 104 electrodes. In various embodiments, wires 110, 112 or their equivalents may have low ohmic resistance or impedance, e.g., less than about 100 ohms. In some embodiments, wires or their equivalents may have an ohmic resistance or impedance greater than about 100 ohms. Wires or their equivalents may have remotely located connectors 120 which provide for the application of voltage or current waveforms to wires. In various embodiments, wires or their equivalents provide electrical connection between one or more electrode pairs and one or more connectors. In some embodiments, voltage waveforms V₁, V₂ are applied to wires or their equivalents.

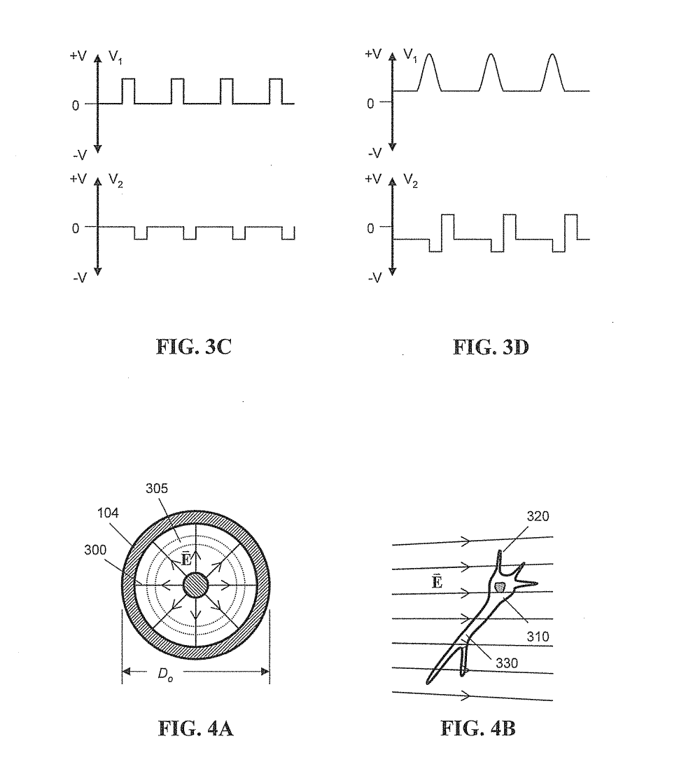

In various embodiments, voltage waveforms V₁, V₂ applied to connectors 120 are transmitted to electrodes 102, 104 and create an electric field in the excitation region 150 between the electrodes. Voltage waveforms V₁, V₂, or current waveforms I₁, I₂, may be derived from various types of electronic equipment known to those skilled in the art of electronics, e.g., they may be produced by a function generator, a waveform generator, a programmable waveform generator, a computer combined with data-acquisition hardware, a computer combined with an analog-to-digital (A/D) board which can convert digital input from the computer's processor to analog output. In certain embodiments, one or both of the voltage waveforms V₁, V₂ will be time varying, and their shape may be selected or programmed by a user. Examples of voltage waveforms that may be applied to the connectors 120 and transmitted to the electrodes 102, 104 are depicted in FIGS. 3A-D. Applied waveforms V₁, V₂ may be characterized by one or more peak voltages V₁ₚ₁, V₂ₚ₁, V₂ₚ₂, voltage offsets, waveform shapes, rates of change of voltage, and repetition rate or periodicity. In some embodiments, one of the waveforms V₁ or V₂ may be held substantially constant at a reference potential, e.g., 0 volts, 0.5 volt, −1 volt, as is depicted in FIGS. 3A-B. In some embodiments, both waveforms may be time varying, as is depicted in FIGS. 3C-D. It will be appreciated that any of a wide variety of waveforms, having different voltage offsets, amplitudes, shapes, rates of change, and periodicity, can be applied to the electrode pair 100.

In various embodiments, application of voltage waveforms to an electrode pair creates time-varying electric fields within an excitation region 150 substantially between electrodes 102 and 104. When electrodes are placed in contact with, or in close proximity to, a well containing neuronal cells in medium, an electric field can couple into the medium and affect movement of ions within the medium, within the cells, across cell membranes, or any combination thereof. Presence of an electric field in any region is accompanied by a gradient in electrical potential in that region. Time-varying gradients in electrical potential within a well comprising a neuronal cell culture and/or movement of ions can alter transmembrane potentials in neuronal cells and stimulate firing of axons and corresponding synaptic vesicle cycling. In various embodiments, stimulation of synaptic vesicle cycling is controlled by voltage or current waveforms applied to an electrode pair 100.

A wide range of voltage or current waveforms may be applied to an electrode pair. In general, any voltage or current waveform within a range between where the stimulation triggers action potentials and where it electroporates the cells or is otherwise toxic can be used in accordance with the present invention. In some embodiments, the magnitude of the electric field [symbol] created within a well comprising a neuronal cell culture by an electrode pair 100 may vary spatially over macroscopic areas, and yet be substantially uniform over microscopic areas, as depicted in FIGS. 4A-B. The drawings of FIGS. 4A-B are representative of an instantaneous electric field. In FIGS. 4A-B, lines 300 represent the direction of electric field [symbol] at a moment in time, and the density of lines represents the magnitude of the electric field at that moment in time. As depicted in FIG. 4A, the electric field's magnitude may vary spatially over the macroscopic region within the excitation region. For example, the density of lines is less near the outer conductor 104 than near the center conductor, so that the electric field is weaker near the outer conductor than near the center conductor. As depicted in FIG. 4B, the electric field's magnitude may be substantially uniform spatially over a microscopic region within the vicinity of a single cell.

Various electrodes can be used in accordance with the present invention. Exemplary electrode design are shown in FIGS. 2A-B and FIGS. 5A-H. For example, a round electrode design may be used for its ease of construction and application. One useful feature of round electrode pairs as shown in FIGS. 2A-B is that the effects of a range of electric field values on cells within the excitation region 150 can be observed simultaneously. For a given set of voltage values V₁, V₂ applied to the electrodes 102, 104 at an instant in time, the electric field will have a range of values within the excitation region 150, the values being highest near the center conductor 102 and lowest near the outer conductor 104, as indicated by FIG. 4A. By recording data from plural annular regions 305 within the excitation region 150, simultaneous observation of the effects of electrical field excitation on cells, at different electric field values, is possible.

In various embodiments, the instantaneous magnitude of the electric field [symbol] within a well comprising a neuronal cell culture or in the vicinity of neuronal cells located in an annular region 305 within the excitation region 150 may vary over time between about 0 volts per centimeter (V/cm) and about 5 V/cm. As used herein, instantaneous magnitude of the electric field is defined as the absolute value of electric field at one instant in time. In some embodiments, instantaneous magnitude may vary temporally between about 0 V/cm and about 10 V/cm. In some embodiments, instantaneous magnitude may vary temporally between about 0 V/cm and about 20 V/cm. In some embodiments, instantaneous magnitude may vary between about 0 V/cm and about 30 V/cm. In some embodiments, instantaneous magnitude may vary between about 0 V/cm and about 50 V/cm. In some embodiments, instantaneous magnitude may vary between about 0 V/cm and about 75 V/cm. In some embodiments, instantaneous magnitude may vary between about 0 V/cm and about 100 V/cm. In some embodiments, instantaneous magnitude may vary between about 0 V/cm and about 150 V/cm. In some embodiments, instantaneous magnitude may vary between about 0 V/cm and about 200 V/cm. In general, instantaneous magnitude of the electric field at a given location within the well will be determined in part by the electrodes' geometrical configuration and electrical properties of the neuronal cell culture medium, as well as proximal materials. In various embodiments, voltage or current waveforms applied to electrodes 102, 104 are controlled to produce a desired instantaneous magnitude and time-varying profile of electric field within the well comprising neuronal cells.

In some embodiments, peak value of a waveform, e.g., peak voltage, peak voltages, peak current, or peak currents, applied to the electrodes may be determined based upon the performance of the instrument and the synaptic vesicle cycle parameter being measured. For example, square waves may be used in accordance with the invention. Suitable square waves may have peak amplitude ranging between 0 and 100V, peak current ranging between 0 and 100 mA, repetition rate ranging between 0 and 100 Hz, and duration of electrical stimulation ranging between 0 and 1 minute. Exemplary parameters suitable for the invention are shown in Table 1.

In certain embodiments, stimulation of action potentials in axons may be determined from fluorescence microscopy (Ryan and Smith, 1995, Neuron, 14:983; incorporated herein by reference). In some embodiments, the peak value applied to electrodes in an electrode pair may be increased until a desired biophysical result is observed, e.g., a fluorescent signal indicative of action potential firing in the neuronal cells is observed. For the electrode pair depicted in FIGS. 2A-B, the desired result may first appear near the inner electrode 102. In some embodiments, the peak value may be increased until the desired result is detected from a desired portion of the excitation region 150, and the peak value selected as a subsequent operating value. In certain embodiments, the peak value may be increased by an excess amount above a “threshold value,” i.e. a value when the desired biophysical result is first observed, and the peak value used as a subsequent operating value.

It will be appreciated that the electrodes may be formed in a wide variety of geometrical configurations. Examples of various designs for electrode pairs are shown in FIGS. 5A-H, wherein electrodes have a distal end 490 and intervening excitation region 450. Conductive wires and connectors are omitted from the illustrations to simplify the drawings. In various embodiments, an electrode pair, e.g., electrodes 402a, 404a, may be dipped into a well comprising cells and medium. In some embodiments, electrodes 402a, 404a may comprise pins or rods. In some embodiments, an electrode pair may comprise a rod 402c and a thin plate 404c, or parallel plates 402e, 404e. As depicted in FIGS. 5G-H, an electrode pair may comprise multiple electrode elements 402g, 404h, e.g., multiple pins, rods, or plates, or any combination thereof.

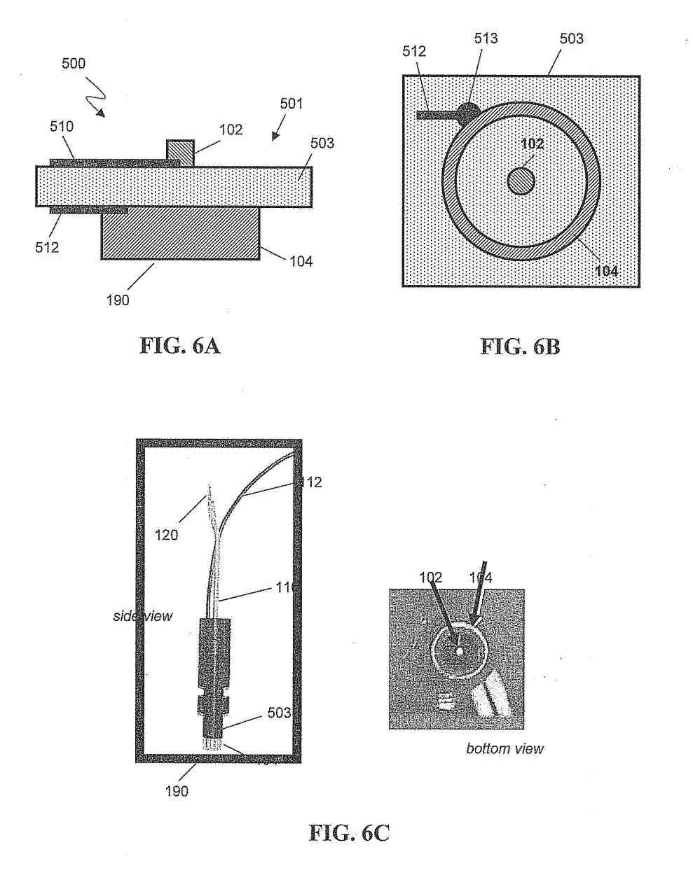

In various embodiments, at least one electrode pair is incorporated into an electrode assembly 500, as depicted in FIGS. 6A-B. Electrodes 102, 104 may be held in place or supported by solid material 503. In certain embodiments, solid material 503 is non-conductive, e.g., a polymer or plastic. In some embodiments, material 503 is transparent, so that placement of the electrodes may be viewed from the top side 501. In some embodiments, material 503 is opaque, so that it blocks light. The electrodes 102, 104 may be affixed to material 503 by glue, thermal bonding, press-fitting, or any combination thereof. In various embodiments, conductive traces 510, 512 may be disposed on the material 503, and electrically connected to the electrodes 102, 104. An electrical connection 513 may be established between a trace and an electrode by solder, conductive glue, or pressure contact. In some embodiments, traces 510, 512 are on opposite sides of material 503. In some embodiments, traces 510, 512 may be located on the same side of material 503. For example and for the electrode assembly depicted in FIGS. 2A-B, the arrangement of traces 510, 512 shown in FIG. 6A may be more practical for fabrication purposes. For the electrode configurations shown in FIGS. 5A-H, conductive traces may be located on the same side of supporting material 503, or on opposite sides of the material.

One particular exemplary embodiment of an electrode assembly is shown in the photo of FIG. 6C. The assembly has an electrode pair, similar to that depicted in FIGS. 2A-B, located at its distal end 190. The outer electrode 104 is visible in the photo. Cylindrically-shaped solid material 503 supports an inner electrode 102 and the outer electrode 104. Wires 110 and 112 connect with the inner and outer electrodes, respectively. A connector 120 is visible in the photo at the end of one wire.

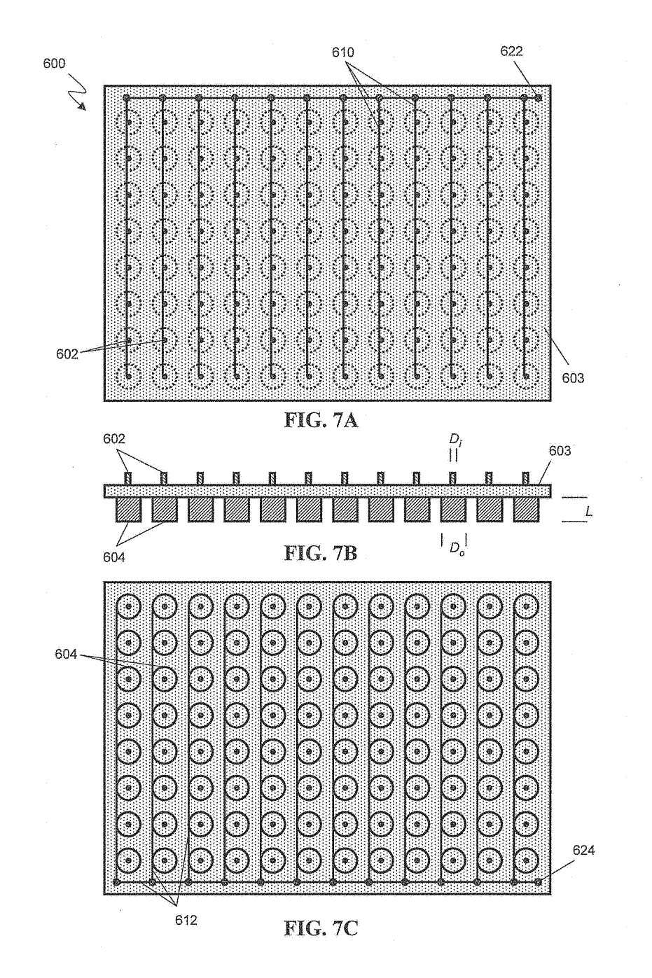

Referring to FIGS. 7A-C, in various embodiments, a plurality of electrode pairs 602, 604 are arranged in a multi-electrode assembly 600 adapted for use with multiwell plates, e.g., 24-well plates, 96-well plates, 384-well plates. In some embodiments, a multi-electrode assembly comprises an electrode pair for each well of the corresponding multiwell plate. In some embodiments, a multi-electrode assembly comprises fewer electrode pairs than the number of wells of the corresponding multiwell plate. Electrodes 602, 604 may be supported by a plate 603 of non-conductive material. Conductive traces 610 may be disposed on the top surface of the plate 603 and provide electrical contact to one electrode 602 of each pair of electrodes. All traces 610 may be electrically connected and further connected to a connection fixture 622, e.g., a contact pad, a plug receptacle, a BNC jack, so that application of a waveform to the fixture 622 provides for substantially simultaneous application of the waveform to all electrodes 602. Conductive traces 612 may be disposed on the bottom surface of the plate 603, and provide electrical contact to the mating electrode 604 of each pair of electrodes. All traces 612 may be electrically connected and further connected to a connection fixture 624, so that the application of a waveform to the connection fixture 624 provides for substantially simultaneous application of the waveform to all electrodes 604.

In some embodiments, a ground plane, or conductive layer of material, may be used to establish connection to one of the electrodes 602 or 604 in each electrode pair instead of individual traces. As an example, one side of the plate 603 may have a thin metallic or conductive film deposited thereon, one of the electrodes 602 or 604 may be soldered to the thin film. In some embodiments, a thin metallic or conductive film may be deposited on both sides of the plate 603 wherein the film on one side is electrically connected to each inner electrode 602 and the film on the opposite side is electrically connected to each outer electrode 604. In such an arrangement it may be necessary to remove, e.g., by drilling or machining, an area of film around each inner electrode on one side of the plate 603 so that the inner and outer electrodes are not shorted by the film.

In various embodiments, electrode pairs 602, 604 are arranged so that all may be dipped simultaneously into a well comprising cells and medium within wells of a multiwell plate. For example, the center-to-center spacing of the electrode pairs will substantially match the center-to-center spacing of wells on a multiwell plate. In various embodiments, the extension L of electrodes 602, 604 beyond the lower surface of the plate 603 will permit the distal end 690 of the electrode pairs to contact, or come into close proximity, with the bottom of each well of a multiwell plate. In various embodiments, the diameter Dₒ of the outer electrode 604 will be sized to substantially match or be less than the inner diameter of a well in the multiwell plate. In certain embodiments, the diameter Dᵢ of the inner electrode will be about 1 mm, and the diameter Dₒ of the outer electrode will be about 6 mm. In certain embodiments, the diameter Dᵢ of the inner electrode will be about 1 mm, and the diameter Dₒ of the outer electrode will be about 3 mm.

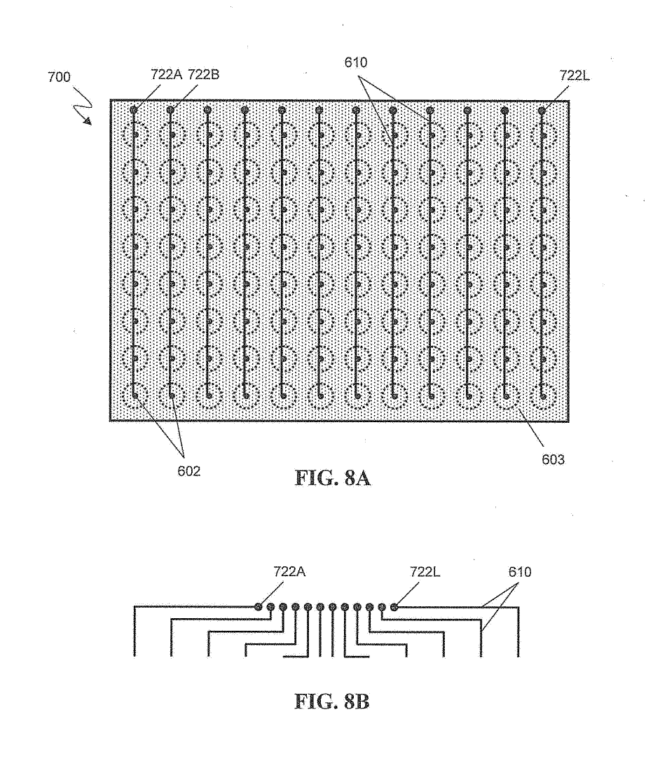

In some embodiments, each column of electrode pairs may be activated independently of other columns. The multi-electrode assembly 700 depicted in FIG. 8A represents an embodiment providing for independent activation of columns of electrode pairs. For this embodiment, each column of electrode pairs may be activated independently by the application of a waveform to one of the connection fixtures 722A-722L. It will be appreciated that the traces 610 may be oriented along rows, instead of columns, so that rows of electrode pairs may be activated independently of other rows.

In some embodiments, connection fixtures 722A-722L may be distributed across at least a portion of the support plate 603. In some embodiments, conductive traces 610 may be patterned so that connection fixtures 722A-722L are localized as depicted in FIG. 8B. In some embodiments, localized connection fixtures may be electrically connected to a standardized electrical connector, e.g., a multi-pin male or female connector, which is firmly mounted on the support plate 603. Use of a standardized connector would reduce set-up time and facilitate interchange of multi-electrode assemblies 600, 700.

In some embodiments, a multi-electrode assembly 600, 700 and multiwell plate may be mounted on a positioning device. The positioning device may place the multi-electrode assembly and multiwell plate at selected locations so that data may be recorded from one or more wells sequentially. In some embodiments, the positioning device comprises a motion-controlled stage. In some embodiments, the positioning device is automated, e.g., controlled by a computer, microprocessor, or microcontroller.

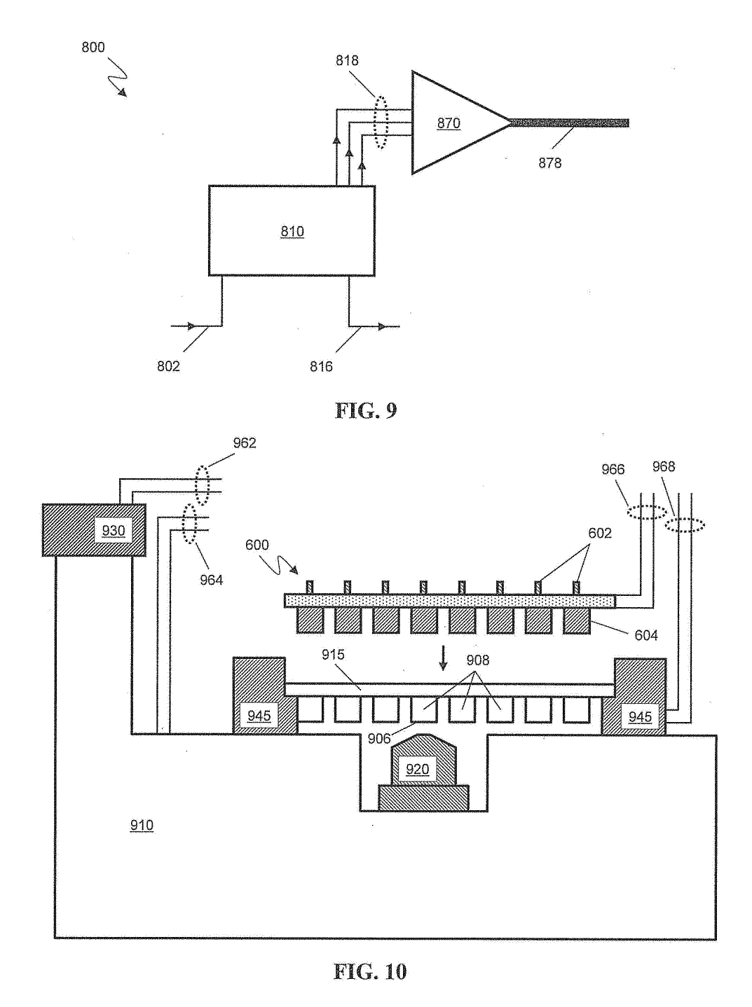

One particular exemplary embodiment of an electronic control system 800 useful for operating multi-electrode assemblies 600, 700 is depicted in the block diagram of FIG. 9. In various embodiments, the electronic control system comprises a waveform generator 810. The waveform generator 810 produces one or more waveform signals 818, which may be transmitted to an amplifying unit 870. The amplifying unit may receive one or more waveform signals, and increase the amplitude, or current, or both amplitude and current in each of the waveform signals. The amplifying unit 870 may then transmit the amplified signals 878 to a multi-electrode assembly 600, 700. The signals 818 fed into the amplifying unit 870 may be provided on multiple separate wires, may be provided via wireless transmission, or may be provided on a bundled multi-wire cable. The signals 878 transmitted from the amplifying unit 870 may be provided on multiple separate wires, may be provided via wireless transmission, or may be provided on a bundled multi-wire cable. Electrical isolation for each signal may be provided at the input of the amplifying unit 870, or may be provided after amplification of the signals. In various embodiments, electrical isolation prevents reflections of applied electrical signals from propagating back to the waveform generator 810. The waveform generator 810 may receive input control electronic signals 802, e.g., timing signals to initiate application of one or more waveforms, amplitude control signals, repetition rate control signals, waveform shape control signals, from an external source. The waveform generator 810 may provide output control electronic signals 816, e.g., timing signals, trigger signals, synchronization signals, to external equipment.

In some embodiments, the waveform generator 810 may be a computer, or a personal computer, or a laptop computer. In some embodiments, the waveform generator 810 may be a stand-alone function generator, a programmable waveform generator, a microprocessor, a microcontroller, or a pulse generator. In some embodiments, waveforms may be generated in software executed on a computer, microprocessor, or microcontroller, which controls the voltage or current at one or more output ports of the computer, microprocessor, or microcontroller. In some embodiments, the waveform generator 810 may comprise a computer, microprocessor, or microcontroller combined with a multi-port data-acquisition device or multi-port analog-to-digital board.

In certain embodiments, an electrical stimulation system comprises a modified commercially-available electroporation system. As an example, a CellAxessHT system, available from Cellectricon (Moldnal, Sweden) may be adapted to excite action potentials in neuronal cells within wells of a multiwell plate. The CellAxessHT system is designed for electroporation of cells cultured, suspended, and/or otherwise contained within wells of 384-well plates. For electroporation of cells, an electric field is applied in the vicinity of the cells wherein the magnitude of the electric field is great enough to create hydrated pathways through the membrane of the cell. The CellAxessHT system has a multi-electrode assembly which can electroporate cells in 96 wells of a 384-well plate simultaneously. In certain embodiments, the CellAxessHT multi-electrode assembly is used unaltered to provide stimulation waveforms to neuronal cells within wells of 384-well plate assays. In certain embodiments, the CellAxessHT multi-electrode assembly is altered, e.g., electrode size, spacing, and/or configuration changed, to provide stimulation waveforms to neuronal cells within wells of 96-well plate assays.

In certain embodiments, and as an additional example, a modified CellAxess CX3 electroporation system, available from Cellictricon AB may be used to electrically stimulate neuronal cells within wells. In various embodiments, the modified CX3 system comprises three pairs of modified electrodes and low auto-fluoresence plastic components. The electrodes can provide substantially uniform fields within their excitation regions. In various embodiments, the electrodes are adapted to fit within wells of 96-well plates, and substantially simultaneously stimulate synaptic vesicle cycling in neuronal cells within the wells. The CX3 system can provide excitation voltage waveforms with peak voltage values ranging from about 1 volt to about 100 volts.

Alternative Stimulation Systems

In certain embodiments, acoustic or ultrasonic stimulation is used to excite action potentials in neuronal cells. In some embodiments, acoustic stimulation comprises one or more sonic or ultrasonic pulses applied to a (one or more) neuronal cell(s) s within wells of multiwell plates. In certain embodiments, an acoustic transducer may be brought into contact or close proximity with a well comprising a (one or more) neuronal cell(s). In some embodiments, an array of acoustic transducers may be spatially matched to an array of wells comprising neuronal cells, and the array of transducers brought into contact or close proximity with an array of wells comprising neuronal cells. In certain embodiments, the array of transducers need not spatially match the array of wells comprising neuronal cell cultures. For example, transducers in the transducer array may spatially match every second, third, or fourth well in an array of wells comprising neuronal cells. In some embodiments, the acoustic transducers may be disposed in a linear array, e.g., an array corresponding to a row or column of a multiwell plate. In various embodiments, ultrasonic pulses initiate the firing of action potentials in the neurons. Examples of acoustic or ultrasonic stimulation systems can be found at the website arraytherapeutic.com/research/index.

In certain embodiments, action potentials are excited in neuronal cells via optical methods. In some embodiments, light-gated ion channels such as channelrhodopsin may be expressed in neuronal cells. Pulses of light may then be used to activate the light-gated ion channels, depolarize the neuron, and initiate action potentials. In some embodiments, firing of the action potentials is carried out at a high repetition rate by exposing cultured, suspended, and/or otherwise prepared neurons with light-gated ion channels to a rapid train of optical pulses. The optical pulses may be provided from one or more luminous systems, e.g., light-emitting diodes, diode lasers, incandescent lamps, neon lamps. In various embodiments, radiation from the one or more luminous systems provides a wavelength to which the light-gated ion channels are responsive. In various embodiments, radiation emitted by the one or more luminous systems is directed toward one or more neuronal cells. Lenses, optical filters, mirrors or other optical components may concentrate and/or direct the emitted radiation onto the one or more neuronal cells.

In some embodiments, an array of luminous devices, and accompanying optical components, may be spatially matched to an array of wells comprising neuronal cells, and the array of luminous devices adapted to illuminate an array of wells comprising neuronal cell. In certain embodiments, the array of luminous devices need not spatially match the array of wells comprising neuronal cells. For example, luminous-device array may spatially match every second, third, or fourth well comprising neuronal cells. In some embodiments, the luminous devices may be disposed in a linear array, e.g., an array corresponding to a row or column of a multiwell plate. In various embodiments, optical pulses from the array of luminous devices initiate the firing of action potentials in the neurons within the neuronal cells.

In certain embodiments, a light-activated glutamate receptor LiGluR is used to initiate action potential firing. In various aspects, a neuronal cell includes a modified kainate receptor with extracellular cysteine, to which a light-activated ligand can bind. Illumination of the tethered ligand at a selected wavelength can flip or switch the ligand's conformation. When photoswitched, glutamate is placed into the binding pocket of the receptor, which can initiate the firing of an action potential. In various embodiments, one or more luminous devices are used to photoswitch the light-activated ligand. The luminous devices may be disposed in an array and comprise additional optical components, as described in relation to an above-described embodiment employing light-gated ion channels. In certain embodiments, LiGluR and photoswitch activation are used to induce high frequency firing of action potentials in neuronal cells.

In some embodiments, photoconductive stimulation may be used to induce action potentials in neurons. Photoconduction is a technique that allows the rapid, non-invasive depolarization of excitable cells, such as neurons. Typically, photoconductive stimulation can be used to induce action potentials in neurons including those grown in culture. Cells may be grown in industry standard multi-well plates, and/or may then be mounted in a reusable dish for live observation under a microscope. The electronics provided can be used to depolarize the cells at user-definable frequency and intensity. Typically, photoconductive stimulation does not involve a direct physical link to the cell (such as an electrode or transistor contact). Examples of photoconduction can be found at the website membrasys.com/techynology.asp.

Detection Technology

In various embodiments, a synaptic vesicle cycling platform includes a detection system for monitoring activity relating to synaptic vesicle cycling. In some embodiments, a detection system provides imaging of neuronal cells, e.g., within a plurality of wells. In some embodiments, a detection system provides monitoring of light emission from at least a region within well comprising neuronal cells. In various embodiments, a detection system communicates with the electrical stimulation system, so that stimulation and detection of synaptic vesicle cycling may be substantially synchronized.

In certain embodiments, a synaptic vesicle cycling assay has one or more of the following characteristics: (a) the assay is a fluorescence-based kinetic assay, e.g., the amount of fluorescence from a well comprising a neuronal cell may vary with time during synaptic vesicle cycling activity; (b) signals from reporters are unamplified, which might provide weaker signals than typical for high-throughput screening assays; and (c) synaptic vesicle cycling is activated by a controlled stimulation system. In various embodiments, a detection system for a synaptic vesicle cycling system is capable of detecting low-level, time-varying, fluorescent signals from one or more neuronal cells, and is physically incorporated as part of the system such that it is operable in combination with an electrical excitation system. In some embodiments, a detection system may provide capturing, transmission, and/or analysis of detected signals. Analysis of detected signals may be completed in about one minute in certain embodiments.

The platform may also include a detection system that comprises at least one detector. Typically a detector comprises an optical sensor configured to detect a luminescent signal from a well. A detector may also comprise an objective lens configured to collect a luminescent signal from a well. An objective lens may be operably linked to the optical sensor, e.g., a charge-coupled device camera, etc. The objective lens may be an air objective lens. Alternatively, the objective lens may be a water or oil immersion lens.

Typically, air objective lenses have lower numerical apertures (NAs) than water or oil immersion lenses. Because NA directly controls the fraction of emitted photons that are collected by the objective, air objectives typically collect less photons than water or oil immersion lenses. Applicants have discovered that certain for applications, e.g., high-throughput platforms, that may comprise the use of one or more air objective lenses, the low collection efficiency associated with the objective can be compensated for in a variety of ways. For example, low collection efficiency associated with the air objective can be compensated by the increasing in the number of synapses imaged in a field, e.g., by increasing neuronal cell number, by increasing the field area size, etc., and/or by increasing the efficiency and specificity of reporter protein expression. Accordingly, applicants have discovered that aspects of synaptic vesicle cycling can be detected in a high-throughput manner with high sensitivity, e.g., aspects of synaptic vesicle cycling in response to as low as 5 to 10 action potentials in neuronal cells can be detected, using one or more low numerical aperture air objective lenses.

The objective lens may have a magnification of 5×, 10×, 20×, 40×, 60×, or 100×. Other appropriate magnifications will be apparent to the skilled artisan based on the teachings of the instant disclosure. The objective lens may have a numerical aperture in a range 0.1 NA to 1.4 NA, 0.1 NA to 1.3 NA, 0.1 NA to 1.2 NA, 0.1 NA to 1.1 NA, 0.1 NA to 1.0 NA, 0.1 NA to 0.9 NA, 0.1 NA to 0.85 NA, 0.1 NA to 0.8 NA, 0.1 NA to 0.75 NA, 0.1 NA to 0.7 NA, 0.1 NA to 0.65 NA, 0.1 NA to 0.6 NA, 0.1 to 0.55 NA, 0.1 to 0.5 NA, 0.1 to 0.45 NA, 0.1 to 0.4 NA, 0.1 NA to 0.35 NA, 0.1 NA to 0.3 NA, 0.1 NA to 0.25 NA, 0.1 NA to 0.2 NA, or 0.1 NA to 0.5 NA. The objective lens may have a numerical aperture of about 0.1 NA, about 0.15 NA, about 0.2 NA, about 0.25 NA, about 0.3 NA, about 0.35 NA, about 0.4 NA, about 0.45 NA, about 0.5 NA, about 0.55 NA, about 0.6 NA, about 0.65 NA, about 0.7 NA, about 0.75 NA, about 0.8 NA, about 0.85 NA, about 0.9 NA, about 0.95 NA, about 1 NA, about 1.1 NA, about 1.2 NA, about 1.3 NA, about 1.4 NA, or more. Other appropriate numerical apertures will be apparent to the skilled artisan based on the teachings of the instant disclosure.

The detector may be configured to detect a luminescent signal from a variety of field areas. The detector may be configured to detect a luminescent signal from a field area in a range of 0.1 mm to 10 mm, 0.1 mm to 9 mm, 0.1 mm to 8 mm, 0.1 mm to 7 mm, 0.1 mm to 6 mm, 0.1 mm to 5 mm, 0.1 mm to 4 mm, 0.1 mm to 3 mm, 0.1 mm to 2 mm, 0.1 mm to 1 mm, or 0.1 mm to 0.5 mm. The objective lens may be configured to collect a luminescent signal from a field area of about 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 6 mm, 7 mm, 8 mm, 9 mm, 10 mm, or more. Other appropriate field areas will be apparent to the skilled artisan based on the teachings of the instant disclosure.

The detection system may comprise a computer operably linked to the detectors, and wherein the computer is configured to transform luminescent signal from the detectors into data characterizing an aspect of synaptic vesicle cycling in a neuronal cell.

In some embodiments of a detection system, synaptic vesicle cycle activity within a neuronal cell may be imaged using optical components, e.g., lenses and filters, and fluorescent emission signals or an image of fluorescent emitters captured on a charge-coupled device (CCD) detector array. The imaging system may detect fluorescent radiation emitted from reporter fluorophores, e.g., GFP tagged to the intra-lumenal domain of synaptic vesicle proteins. A captured image may be of a portion of a well of a multi-well plate. Once an image is captured electronically on a CCD array, the signals may be subsequently processed on a computer executing data-processing software. Images or signals may be captured sequentially to provide kinetic analysis of fluorescence during synaptic vesicle cycling.

A non-limiting example of a detection system that can provide images of activity relating to synaptic vesicle cycling within a well of a multi-well plate is depicted in FIG. 10. In various embodiments, an inverted epifluorescence microscope 910 is used to image fluorescent emission from within a well comprising a neuronal cell. As an example, a Zeiss Axiovert Z1 with an objective lens 920, e.g., a 10×, 0.45 NA air objective lens, can image synaptic vesicle cycle activity within at least a portion of a well 908. In various embodiments, at least a portion of the bottom of each well 906 is transparent, so that neuronal cells within a well can be optically observed through the bottom of the well. Image may be captured on a CCD array 930 coupled to the microscope. The CCD array may be in communication with a computer, so that images may be transferred to, and optionally stored within memory on the computer. Images may be subsequently processed by the computer to extract relevant information relating to synaptic vesicle cycling.

In various embodiments, the imaging system (e.g., microscope 910) permits open access to the wells 908 of the multiwell plate 915 so that a multi-electrode assembly 600 may be placed into the wells from above the plate, as depicted. In various embodiments, the multiwell plate 915 is mounted on a positioning device 945 so that each well of the multiwell plate may be inspected in sequence. The positioning device 945 may be automated and/or computer controlled for convenience, and optionally synchronized with the electrical excitation system, image-capture apparatus and/or data processing system. For automated data capture and processing, the CCD array 930, microscope 910, positioning device 945, and multi-electrode assembly 600 may be in communication with a central controller, e.g., a computer, a microcontroller, a microprocessor, through wired or wireless links 962, 964, 966, 968.

In some applications, a low rate of analysis may be adequate for the needs of the experimenter or user, and elements of an embodiment of a synaptic vesicle cycling system depicted in FIG. 10 may be sufficient for the particular application. For example, in some embodiments, synaptic vesicle cycling extends for a time between about 30 seconds and about 150 seconds, e.g., about 30 seconds, about 40 seconds, about 50 seconds, about 60 seconds, about 70 seconds, about 80 seconds, about 90 seconds, about 100 seconds, about 110 seconds, about 120 seconds, about 130 seconds, about 140 seconds, or about 150 seconds. Accordingly, each well may be imaged for any of those durations. In some embodiments, each well may be imaged for a period of time shorter than this duration, for example, about 5 seconds, about 10 seconds, about 20 seconds, about 30 seconds, or about 40 seconds. Using a single microscope, inspecting one well at a time, and assuming the data processing time is negligible compared to the imaging time, data capture and processing time for a 96-well plate may be between about 1 hour and about 4 hours, e.g., about 1 hour, about 1.5 hours, about 2 hours, about 2.5 hours, about 3 hours, about 3.5 hours, or about 4 hours. If a higher rate of analysis is required, a parallel detection system may be used.

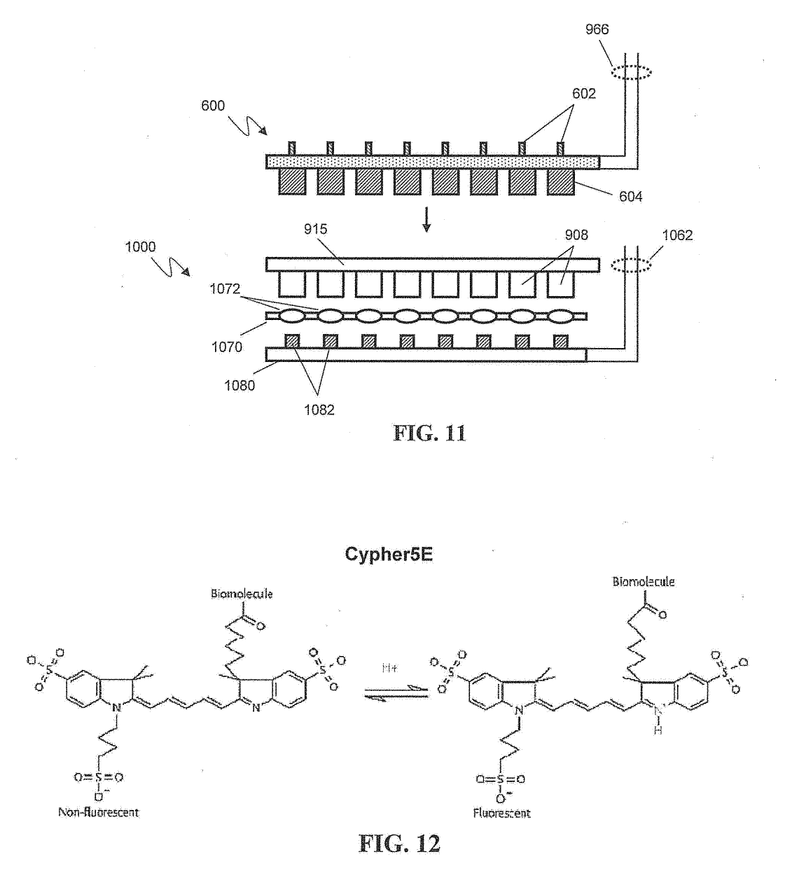

FIG. 11 depicts a parallel detection system 1000 which may be used for an embodiment of a high-throughput synaptic vesicle cycling system. In various embodiments, a parallel detection system 1000 comprises a lens array 1070 and a detector array 1080. A lens array 1070 may contain an array of lenses 1072 which may be distributed in one dimension, e.g., in a configuration corresponding to plural wells in a row or column of wells 906, or may be distributed in two dimensions, e.g., in a configuration corresponding to plural wells in rows and columns of wells. In some embodiments, lenses of the lens array 1070 may be spatially matched to the location of each well in a multiwell plate. The lens array 1070 need not have a lens spatially matched to each and every well in a row and/or column. For example, a lens may be matched to every other well in a row and/or column, or every third well, every fourth well, every fifth well, or every sixth well. When a lens is not matched to every well, the multiwell plate 915 may be moved to adjacent unread wells between readings.

A detector array 1080 may contain an array of photodetectors 1082. Photodetectors 1082 may be high-sensitivity detectors, e.g., photomultiplier tubes, avalanche photodiodes, silicon drift detectors, etc. Optical filters may be placed over the detectors, e.g., to reduce radiation from wavelengths different than that of fluorescent emission from fluorophore-tagged specimens within the wells 906. In some embodiments, detectors in the photodetector array 1080 may be spatially matched to the location of each well in a multiwell plate. In some embodiments, photodetectors 1082 may be distributed in a configuration matched to lenses 1072 in the lens array 1070. In various embodiments, each lens 1072 collects fluorescent emission radiating from at least a portion of a well 906 and directs the collected emission to a corresponding photodetector 1082. Signals from each photodetector 1082 may be transmitted through wired or wireless links 1062 to a central controller (e.g., a computer, a microprocessor, a microcontroller, etc.) for subsequent processing. In some embodiments, each photodetector 1082 may be optically isolated from adjacent or nearby wells with optical baffles or opaque material, so that each photodetector only receives fluorescent radiation from a well directly opposed to its corresponding collecting lens 1072. In various embodiments, the lenses 1072 may be coated with anti-reflective coatings and/or band-pass coatings.

In some embodiments, plate readers that are capable of imaging multi-wells (e.g., 4, 16, 24, 48, 96, 384 wells) in parallel can be used for this system. For example, a commercially available plate reader (e.g., the plate::vision system available from Perkin Elmer, Waltham, Mass.) may be used as a parallel detection system. This plate reader is capable of kinetic-based fluorescence analysis, and allows for access to the top of a multiwell plate for integration of a multi-electrode assembly. The plate::vision system has high collection efficiency optics and has special optics designed for the analysis of 96 wells in parallel. The plate::vision plate system was evaluated by the inventors for its ability to record the synaptic vesicle cycle in multiple wells in parallel and is described in detail in the Examples section. Additional suitable parallel plate readers include but are not limited to the FLIPR^{TETRA®} (Molecular Devices, Union City, Calif.), the FDSS7000 (Hamamatsu, Bridgewater, N.J.), and the CellLux (Perkin Elmer, Waltham, Mass.).

In various embodiments, signals received from an imaging system as depicted in FIG. 10, or a parallel detection system as depicted in FIG. 11, are processed to evaluate synaptic vesicle cycling or synaptic vesicle activity as a function of time. In certain embodiments, a signal representative of synaptic vesicle cycling, e.g., a fluorescent signal, is recorded as a function of time. The intensity and/or repetition rate of the signal may be recorded. For an embodiment comprising an imaging system, plural signals may be recorded for one neuronal cell culture, wherein each signal corresponds to a particular location, or neuron, within the culture. For an embodiment comprising an imaging system, the signals from plural cells may be combined to obtain a total, or average signal, for the well comprising neuronal cells.

In various embodiments, signals representative of synaptic vesicle cycling or neural activity may be evaluated as readouts of synaptic vesicle cycling assays of the invention. In some embodiments, electrophysiological analysis of synaptic transmission may be used as a readout of inventive assays in accordance with the present invention. For example, signals representative of synaptic fatigue can be used as a readout in inventive assays of the present invention to identify compounds that affect synaptic transmission. In certain embodiments, signals, e.g., fluorescence signals, representative of synaptic vesicle cycling or neural activity may exhibit oscillations, ripple, certain waveform shapes, exponential trajectories, e.g., saturation or recovery, rate of increase (e.g., rate of rise of fluorescence), rate of decrease (e.g., rate of decay of fluorescence), and/or noise-like characteristics. In various embodiments, the signals may be analyzed to quantify the observed characteristics. As an example, a signal representative of synaptic vesicle cycling or neural activity may exhibit an exponential decay. For such a signal, a mathematical expression containing an exponential decay, e.g., exp(−t/τ), where t represents time and τ represents a characteristic decay constant, may be fit to the observed signal to obtain a measure of τ.

Interfacing of the Stimulation and Detection Systems

In various embodiments, an electrical stimulation system and detection system are interfaced and incorporated into a synaptic vesicle cycling system, as depicted in FIG. 10 (exemplary depiction of a low-throughput platform) and FIG. 11 (exemplary depiction of a high-throughput platform). A multi-electrode assembly 600, its associated electronic equipment, and a detection system may be interfaced to a central controller (e.g., a computer, a microprocessor, a microcontroller, etc.) through communication links 966, 962, 964, 968, 1062, so that experimental procedures, data collection, and data processing may be substantially automated or semi-automated. In various embodiments, electrodes 602, 604 of a multi-electrode assembly 600 are placed into contact with neuronal cell cultures within wells 906 of a multiwell plate 915 from a direction above the multiwell plate, and a detection system is located below the wells. As an example of an automated experimental procedure in which the electrical stimulation system and detection system are interfaced with a central computer, the computer may execute the following steps: (a) issue electronic commands to positioning apparatus 945 to position a first well over a microscope objective lens 920, (b) issue commands to initialize microscope operational parameters, e.g., select lens, filter settings, (c) initialize CCD array, e.g., power setting, gain setting, electronic shutter rate, (d) begin receiving data from the CCD array, (e) issue electronic commands to the electrical stimulation system to apply one or more waveforms to the electrodes within the first well, (f) issue commands to terminate the application of waveforms, (g) end receiving data from the CCD array, and (h) issue electronic commands to position apparatus 945 to another well and repeat cycle (b-h).

In certain aspects, an interfaced system may be used to evaluate the ability of various electrode designs to trigger synaptic vesicle cycling within neuronal cells, for example, as exemplified in the Examples section. As a non-limiting example, primary neurons in culture within a 96-well plate are infected with a synaptopHluorin, vGlut1-pHluorin-, or synaptophysin-pHluorin-expressing adeno-associated virus. After a culture period, culturing media may be replaced with an assay buffer. The plate may then be placed in a synaptic vesicle cycling system, and electrodes placed into the wells. Voltage waveforms are applied, and at least a portion of the fluorescence generated by synaptopHluorin will be directed to the detection apparatus and recorded (e.g., as an image, a sequence of images, an intensity level, and/or sequence of intensity values). Imaging of activity relating to synaptic vesicle cycling within one or more cell cultures may be carried out with an inverted microscope, as depicted in FIG. 10. Detection of time-varying fluorescence signals may be performed with the inverted microscope or lens and photodetector assembly, as depicted in FIG. 11. Stimulation may be repeated for different electrode designs and resulting data compared to evaluate the effectiveness of various electrode designs.

Platforms

In some embodiments, synaptic vesicle cycling assays in accordance with the invention are carried out on low-throughput platform. Typically, low throughput assay systems use better optics (e.g., epifluorescence microscopes) and provide better resolutions. As a result, in some embodiments, low throughput platform are capable of characterizing individual synapses. Typically, low throughput platforms are particularly useful for characterizing the effects of individual compounds. For example, a known drug that treats a particular disorder can be analyzed using low throughput platforms to determine if it affects synaptic vesicle cycling. An exemplary low throughput platform is depicted in FIG. 10.

High Throughput Platforms

In some embodiments, synaptic vesicle cycling assays in accordance with the present invention can be carried out on high throughput platforms. High throughput platforms are particularly useful for blind screenings to identify synaptic vesicle cycling modulators (e.g., stimulators and/or inhibitors) from a large number of candidate or test agents. In certain embodiments, high throughput screening assays may include steps of: (a) providing a plurality of neuronal cells as described herein, (b) providing a plurality of test agents such that members of the plurality of test agents contact each of the plurality of neuronal cells, (c) conducting synaptic vesicle cycling assays as described herein, and (d) determining if any members of the plurality of test agents modulate one or more activities relating to synaptic vesicle cycling relative to a control.

In some embodiments, the plurality of neuronal cells are provided in a plurality of wells. For example, multiwell plates, e.g., 24-, 48-, 96-, or 384-well plates, may be used for high throughput assays. An exemplary high throughput screening system is depicted in FIG. 11.

In order to induce synaptic vesicle cycling in multiwells, high throughput platform typically include stimulation systems to create field potentials in the wells. In some embodiments, the wells of the plates are accessible from the top in order to integrate the stimulation technology into the assay system. In some embodiments, custom developed multi-well electrical stimulators can be used. In some embodiments, multi-well electrical stimulators suitable for the invention contain multiple electrode pairs that can be placed into wells comprising a plurality of neuronal cells. Electrodes may comprise an inner rod-like conducting electrode surrounded by an outer, cylinder-like conducting electrode. The stimulation step may comprise applying one or more excitation voltage waveforms to the electrodes, such that an electric field is created within an excitation region within the well between the electrodes of an electrode pair. In various embodiments, the stimulation step comprises inducing synaptic vesicle cycling by the application of stimulation waveforms.

In some embodiments, suitable stimulation systems can be modified from multi-well plate electroporation systems. For example, a commercially-available high-throughput electroporator (e.g., CellAxessHT provided by Cellectricon designed to deliver siRNAs) can be optimized to maximize the electric field potential coverage of the wells of multiwell plates (e.g., 96-well plates) to serve as stimulation systems of high throughput synaptic vesicle cycling assays of the invention. Non-limiting examples of custom modifications of a commercially-available high-throughput electroporator are described in details in the Examples section. Additional stimulation systems suitable for high throughput platforms are described herein.

Typically, high throughput assays in accordance with the present invention are fluorescence-based kinetic assays. Typically, the reporters' signals are unamplified in synaptic vesicle cycling assays, thus, leading to weak signals. Suitable imaging systems have a high photon collection efficiency. A high collection efficiency results in improved signal to noise ratios and reduced bleaching during kinetic reads, as less light is necessary to measurably excite the fluorophore. In various embodiments, imaging/monitoring systems suitable for the invention are capable of monitoring the plurality of wells comprising a plurality of neuronal cells in parallel for signals (e.g., fluorescent emission) indicative of synaptic vesicle cycling. In certain embodiments, suitable imaging systems image at least a portion of the well comprising neuronal cells and collect an image or sequence of images of the portion of a well comprising neuronal cells. In certain embodiments, imaging/monitoring systems suitable for the invention collect and detect amounts of signals (e.g., fluorescent radiation) from a plurality of wells comprising a plurality of neuronal cells in parallel. In some embodiments, suitable monitoring/imaging systems are capable of further processing any collected data representative of one or more aspects of synaptic vesicle cycling, e.g., kinetics of various synaptic vesicle cycling processes (e.g., endocytosis, nucleation, invagination, etc.). Typically, suitable imaging systems are capable of performing fluorescence, kinetic analysis on a relatively short time scale (e.g., 1 min).

In some embodiments, a custom or a commercially available high-content screening systems (e.g., Pathway, Becton Dickinson, Franklin Lakes, N.J.; ImageXpress MICRO (Molecular Devices, Union City, Calif.; Opera, Perkin Elmer, Waltham, Mass.; ArrayScan, Thermo Fisher Scientific, Waltham, Mass.) can be used. It is contemplated that these high-content screening systems are capable of performing at least 12 synaptic vesicle cycling assays per hour. In some embodiments, multiwell plate readers are used to make the system amenable to compound discovery by increasing the screening throughput. Suitable multiwell plate readers are capable of imaging multiple wells of a multiwell plate in parallel. Suitable plate readers include commercially available plate readers (e.g., the plate::vision provided by Perkin Elmer). Typically, suitable plate readers are capable of kinetic, fluorescence analysis, allow access to the top of the plate for the integration of a stimulation system, have high collection efficiency optics, and/or special optics designed for the analysis of multiple wells in parallel. Additional suitable imaging systems are described in the detection systems section.

Typically, high throughput platforms involve using a single instrument to measure multiple synaptic vesicle cycle assays simultaneously. It is desirable for a high throughput platform to have the capacity to measure as many components of the synaptic vesicle cycle during as many synaptic vesicle cycle modes as possible. To be able to study the synaptic vesicle cycle during the different modes of endocytosis (e.g., saturating and non-saturating), it is important that the assay system is able to measure the synaptic vesicle cycle reporter signal during and following both low and high intensity stimulation trains. Low intensity stimulation trains (e.g., less than 10 Hz for less than 100 action potentials) induces the mode of non-saturating endocytosis, while high intensity stimulation trains (e.g., greater than 10 Hz for greater than 100 action potentials) induces the mode of saturating endocytosis. In some embodiments, an instrument capable of measuring the synaptic vesicle cycling response (e.g., exocytosis) to at least 50 action potentials delivered at at least 10 Hz is used. This stimulation range allows for the analysis of the synaptic vesicle cycle during both the non-saturating and the saturating endocytosis regime. In some embodiments, a suitable instrument is capable of measuring the synaptic vesicle cycling response to at least 50, 100, 200, 300, or more action potentials delivered at at least 10, 20, 30, 40 or 50 Hz.

In some embodiments, the signal strength of assays of the present invention may be increased by maximizing the number of responding fluorophores per well. For example, the signal strength may be increased by plating neurons at high density, which leads to a higher synapse density. In some embodiments, at least 20,000 neurons/cm² may be plated per well. In some embodiments, at least 50 (e.g., at least 100, 150, 200, 250, 300, 350, 400, 450, 500, 550, 600, 650, 700, 750, 800, 850, 900, 950, 1,000, or more) synapses may be monitored simultaneously.

In some embodiments, the signal strength of assays of the present invention may be increased by increasing the percentage of neurons that express a reporter indicative of synaptic vesicle cycling (e.g., synaptophysin-pHluorin). In some embodiments, at least 90% (e.g., at least 91%, 92%, 93%, 94%, 95%, 96%, 97%, 98%, 99%, 100%) of the neurons utilized in the present invention express a reporter indicative of synaptic vesicle cycling. Various methods may be used to efficiently label the neurons with reporters. For example, viral infection may be used. One exemplary viral expression system suitable for the present invention is based on the adeno-associated virus expression system. Typically, more than ˜90% infection rates can be obtained using the adeno-associated virus expression system. In some embodiments, promoters that can express reporters in both excitatory and inhibitory neurons are used.

In some embodiments, reporter-labeled neurons can be obtained from transgenic animals (e.g., mouse) harboring the reporter gene. Transgenic animals expressing a desired reporter gene (e.g., synaptophysin-pHluorin) can be generated using standard methods known in the art. Transgenic animals provide reliable and highly reproducible labeling and signals.

In some embodiments, the signal strength may be increased by increasing the fraction of functional synaptic vesicles labeled with a reporter (e.g., vGlut1-pHluorin) indicative of synaptic vesicle cycling. For example, the fraction of pHluorin-labeled functional synaptic vesicles can be evaluated based on counting the total number of vesicles that can recycle using, e.g., alkaline trapping with the V-type ATPase inhibitor bafilomycin. Stimulation with a few hundred action potentials results in all vesicles that can fuse with the plasma membrane fusing at least once. In the presence of bafilomycin, the pHluorin fluorescence becomes trapped in the elevated state. The maximum fluorescence divided by the quantal size gives the number of vGlut1-pHluorin labeled vesicles at each synapse. This number can be compared to that obtained using FM 1-43 labeling. FM 1-43 is an organic amphipathic fluorescent tracer that will label synaptic vesicles as they undergo endocytosis. The total vesicle pool size may be measured by counting FM1-43-labeled vesicles (Ryan et al. Nature (1997) Jul. 31; 388(6641):478-82, incorporated herein by reference). In some embodiments, at least 40% (e.g., at least 45%, 50%, 55%, 60%, 65%, 70%, 75%, 80%, 85%, 90%, 95%, 100%) of functional synaptic vesicles are labeled with a reporter indicative of synaptic vesicle cycling in accordance with the present invention.

It will be appreciated by one skilled in the art that modifications to instrument sensitivity, reporter signal strength, and other parameters may increase the signal to noise ratio of the assays of the present invention.

Increased signal to noise ratio allows measurement of synaptic vesicle cycling response to fewer action potentials (e.g., 40, 20, 10, 5 or fewer action potentials) at at least 10, 20, 30, 40 or 50 Hz and enabling measurement of additional processes of the synaptic vesicle cycle (e.g., probability of release) in addition to the overall synaptic vesicle cycle. Such modifications and variations of the assays described herein are within the scope of the present invention.

Test Agents

Various candidate agents can be tested for potential modulators (e.g., inhibitors or stimulators) of synaptic vesicle cycling. Exemplary test agents include, but are not limited to, chemical compounds, small molecules, proteins or peptides, antibodies, co-crystals, nano-crystals, microorganisms (e.g., virus, bacteria, fungi, etc.), nucleic acids (e.g., DNAs, RNAs, DNA/RNA hybrids, siRNAs, shRNAs, miRNAs, ribozymes, aptamers, etc.), carbohydrates (e.g. mono-, di-, or poly-saccharides), lipids (e.g., phospholipids, triglycerides, steroids, etc.), natural products, any combination thereof. Candidate agents can also be designed using computer-based rational drug design methods. Typically, a plurality of test agents (e.g., libraries of candidate agents) are tested in screening assays for potential modulators. In some embodiments, test agents are biodegradable and/or biocompatible.

Peptides

In some embodiments, a potential modulator of synaptic vesicle cycling can be obtained by screening a random peptide library produced by recombinant bacteriophage, for example, Scott and Smith, Science, 249:386-390 (1990); Cwirla et al., Proc. Natl. Acad. Sci., 87:6378-6382 (1990); Devlin et al., Science, 249:404-406 (1990), or a chemical library. Using the “phage method” very large libraries can be constructed (10⁶-10⁸ chemical entities). A second approach uses primarily chemical methods, of which the Geysen method (Geysen et al., Molecular Immunology 23:709-715 (1986); Geysen et al. J. Immunologic Method 102:259-274 (1987)) and the method of Fodor et al. (Science 251:767-773 (1991)) are examples. Furka et al. 14th International Congress of Biochemistry, Volume 5, Abstract FR:013 (1988); Furka, Int. J. Peptide Protein Res. 37:487-493 (1991), Houghton (U.S. Pat. No. 4,631,211, issued December 1986) and Rutter et al. (U.S. Pat. No. 5,010,175, issued Apr. 23, 1991) describe methods to produce a mixture of peptides that can be tested as modulators of activities relating to synaptic vesicle cycling.

In some embodiments, synthetic libraries (Needels et al., Proc. Natl. Acad. Sci. USA 90:10700-4 (1993; Ohlmeyer et al., Proc. Natl. Acad. Sci. USA 90:10922-10926 (1993); Lam et al., International Patent Publication No. WO 92/00252; Kocis et al., International Patent Publication No. WO 9428028, each of which is incorporated herein by reference in its entirety), and the like can be used to screen for modulators of synaptic vesicle cycling according to the present invention. Once a potential modulator is identified, chemical analogues can be either selected from a library of chemicals as are commercially available from commercial vendors such as ChemBridge Libraries (chembridge.com), BIOMOL International, ASINEX, ChemDiv, ChemDB, ICCB-Longwood or alternatively synthesized de novo.

Small Molecules

In some embodiments, small molecule libraries, analogues thereof, are screened for modulators of synaptic vesicle cycling. In some embodiments, compound libraries synthesized de novo can be screened to identify novel compounds that have modulatory functions of synaptic vesicle cycling. In some embodiments, public libraries containing drugs (including FDA approved drugs) can be screened to identify existing compounds whose synaptic vesicle cycling modulating activities are previously unknown. In some embodiments, modified libraries containing derivatives or analogues of existing compounds can be synthesized using methods well known in the art and screened to identify novel or improved modulators of synaptic vesicle cycling. Suitable small molecule compound libraries can be obtained from commercial vendors such as ChemBridge Libraries (chembridge.com), BIOMOL International, ASINEX, ChemDiv, ChemDB, ICCB-Longwood. In some embodiments, suitable small molecule libraries contain a large collection (e.g., >100,000 compounds) of commercial compounds selected for diversity and good “drug-like” properties.

Antisense RNAs and Ribozymes

In some embodiments, antisense molecules can be screened for potential modulators of synaptic vesicle cycling. Antisense molecules are RNA or single-stranded DNA molecules with nucleotide sequences complementary to a specified mRNA. When a laboratory-prepared antisense molecule is injected into cells containing the normal mRNA transcribed by a gene under study, the antisense molecule can base-pair with the mRNA, preventing translation of the mRNA into protein. The resulting double-stranded RNA or RNA/DNA is digested by enzymes that specifically attach to such molecules. Therefore, a depletion of the mRNA occurs, blocking the translation of the gene product so that antisense molecules find uses in medicine to block the production of deleterious proteins. Methods of producing and utilizing antisense RNA are well known to those of ordinary skill in the art (see, for example, C. Lichtenstein and W. Nellen (Editors), Antisense Technology: A Practical Approach, Oxford University Press (December, 1997); S. Agrawal and S. T. Crooke, Antisense Research and Application (Handbook of Experimental Pharmacology, Volume 131), Springer Verlag (April, 1998); I. Gibson, Antisense and Ribozyme Methodology: Laboratory Companion, Chapman & Hall (June, 1997); J. N. M. Mol and A. R. Van Der Krol, Antisense Nucleic Acids and Proteins, Marcel Dekker; B. Weiss, Antisense Oligonodeoxynucleotides and Antisense RNA Novel Pharmacological and Therapeutic Agents, CRC Press (June, 1997); Stanley et al., Antisense Research and Applications, CRC Press (June, 1993); C. A. Stein and A. M. Krieg, Applied Antisense Oligonucleotide Technology (April, 1998)).

In some embodiments, antisense molecules and ribozymes suitable for modulating synaptic vesicle cycling pathway can be designed based on sequence information of proteins and genes involved in synaptic vesicle cycling. For example, antisense molecules and ribozymes can be designed to target presynaptic proteins including, but not limited to, intrinsic vesicle proteins (e.g., synaptic vesicle protein 2 (SV2), synaptophysins, synaptotagmins, vesicle-associated membrane polypeptides (VAMPs), neurotransmitter transporters (NT transporters), synaptogyrins, proton pump), peripheral vesicle proteins (e.g., Rabs, cystine string proteins (CSPs), synapsins), synaptic plasma membrane proteins (e.g., calcium channels, synaptosome-associated protein of 25 kDa (SNAP-25), syntaxin), and cytosolic proteins (e.g., SNAPs, n-Sec1), synapsins (e.g., synapsin I, II and III), dephosphins (e.g., dynamin I, PIP5K1γ, and synaptojanin I).

The antisense molecules and ribozymes may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding UGGT. Such DNA sequences maybe incorporated into a wide variety of vectors with suitable RNA polymerase promoters such as T7 or SP6. Alternatively, these cDNA constructs that synthesize antisense RNA constitutively or inducibly can be introduced into cell lines, cells, or tissues.

RNA molecules may be modified to increase intracellular stability and half-life. Possible modifications include, but are not limited to, the addition of flanking sequences at the 5′ and/or 3′ ends of the molecule or the use of phosphorothioate or 2′O-methyl rather than phosphodiesterase linkages within the backbone of the molecule. This concept can be extended by the inclusion of nontraditional bases such as inosine, queosine, and wybutosine, as well as acetyl-, methyl-, thio-, similarly modified forms of adenine, cytidine, guanine, thymine, and uridine which are not as easily recognized by endogenous endonucleases.

Interfering RNAs

RNA interference (RNAi) is a mechanism of post-transcriptional gene silencing mediated by double-stranded RNA (dsRNA), which is distinct from the antisense and ribozyme-based approaches described above. dsRNA molecules are believed to direct sequence-specific degradation of mRNA in cells of various lineages after first undergoing processing by an RNase III-like enzyme called DICER (Bernstein et al., Nature 409:363, 2001) into smaller dsRNA molecules comprised of two 21 nt strands, each of which has a 5′ phosphate group and a 3′ hydroxyl, and includes a 19 nt region precisely complementary with the other strand, so that there is a 19 nt duplex region flanked by 2 nt-3′ overhangs. RNAi is thus mediated by short interfering RNAs (siRNA), which typically comprise a double-stranded region approximately 19 nucleotides in length typically with 1-2 nucleotide 3′ overhangs on each strand, resulting in a total length typically of between approximately 21 and 23 nucleotides.

It will also be appreciated that siRNAs can have a range of lengths, e.g., the double-stranded portion can range from 15-29 nucleotides. It will also be appreciated that the siRNA can have a blunt end or a 3′ overhang at either or both ends. If present, such 3′ overhang is often from 1-5 nucleotides in length.

siRNA has been shown to downregulate gene expression when transferred into mammalian cells by such methods as transfection, electroporation, or microinjection, or when expressed in cells via any of a variety of plasmid-based approaches. RNA interference using siRNA is reviewed in, e.g., Tuschl, T., Nat. Biotechnol., 20:446-448, May 2002. See also Yu, J., et al., Proc. Natl. Acad. Sci., 99(9), 6047-6052 (2002); Sui, G., et al., Proc. Nail. Acad. Sci., 99(8), 5515-5520 (2002); Paddison, P., et al., Genes and Dev., 16, 948-958 (2002); Brummelkamp, T. et al., Science, 296, 550-553 (2002); Miyagashi, M. and Taira, K., Nat. Biotech., 20, 497-500 (2002); Paul, C., et al., Nat. Biotech., 20, 505-508 (2002).

Indeed, in vivo inhibition of specific gene expression by RNAi has been achieved in various organisms including mammals. For example, Song et al., Nature Medicine, 9:347-351 (2003) discloses that intravenous injection of Fas siRNA compounds into laboratory mice with autoimmune hepatitis specifically reduced Fas mRNA levels and expression of Fas protein in mouse liver cells. Several other approaches for delivery of siRNA into animals have also proved to be successful. See e.g., McCaffery et al., Nature, 418:38-39 (2002); Lewis et al., Nature Genetics, 32:107-108 (2002); and Xia et al., Nature Biotech., 20:1006-1010 (2002).

siRNA may include two individual nucleic acid strands or a single strand with a self-complementary region capable of forming a hairpin (stem-loop) structure. A number of variations in structure, length, number of mismatches, size of loop, identity of nucleotides in overhangs, etc., are consistent with effective siRNA-triggered gene silencing. While not wishing to be bound by any theory, it is thought that intracellular processing (e.g., by DICER) of a variety of different precursors results in production of siRNA capable of effectively mediating gene silencing. Generally it is desirable to target exons rather than introns, and it may also be particularly desirable to select sequences complementary to regions within the 3′ portion of the target transcript. Generally it is preferred to select sequences that contain approximately equimolar ratio of the different nucleotides and to avoid stretches in which a single residue is repeated multiple times.

siRNA may thus comprise RNA molecules typically having a double-stranded region approximately 19 nucleotides in length typically with 1-2 nucleotide 3′ overhangs on each strand, resulting in a total length of between approximately 21 and 23 nucleotides. As used herein, siRNA also includes various RNA structures that may be processed in vivo to generate such molecules. Such structures include RNA strands containing two complementary elements that hybridize to one another to form a stem, a loop, and optionally an overhang, preferably a 3′ overhang. Typically, the stem is approximately 19 bp long, the loop is about 1-20, preferably about 4-10, and more preferably about 6-8 nucleotides long and/or the overhang is typically about 1-20, and preferably about 2-15 nucleotides long. In certain embodiments of the invention the stem is minimally 19 nucleotides in length and may be up to approximately 29 nucleotides in length. Loops of 4 nucleotides or greater are less likely subject to steric constraints than are shorter loops and therefore may be preferred. The overhang may include a 5′ phosphate and a 3′ hydroxyl. The overhang may, but need not, comprise a plurality of U residues, e.g., between 1 and 5 U residues.

In some embodiments, siRNAs may be designed based on sequence information of proteins and genes involved in synaptic vesicle cycling. For example, siRNAs can be designed to target genes encoding presynaptic proteins including, but not limited to, intrinsic vesicle proteins (e.g., synaptic vesicle protein 2 (SV2), synaptophysins, synaptotagmins, vesicle-associated membrane polypeptides (VAMPs), neurotransmitter transporters (NT transporters), synaptogyrins, proton pump), peripheral vesicle proteins (e.g., Rabs, cystine string proteins (CSPs), synapsins), synaptic plasma membrane proteins (e.g., calcium channels, synaptosome-associated protein of 25 kDa (SNAP-25), syntaxin), and cytosolic proteins (e.g., SNAPs, n-Sec1), synapsins (e.g., synapsin I, II and III), dephosphins (e.g., dynamin I, PIP5K1γ, and synaptojanin I).

Suitable siRNAs can be synthesized using conventional RNA synthesis methods. For example, they can be chemically synthesized using appropriately protected ribonucleoside phosphoramidites and a conventional DNA/RNA synthesizer. Various applicable methods for RNA synthesis are disclosed in, e.g., Usman et al., J. Am. Chem. Soc., 109:7845-7854 (1987) and Scaringe et al., Nucleic Acids Res., 18:5433-5441 (1990). Custom siRNA synthesis services are available from commercial vendors such as Ambion (Austin, Tex., USA), Dharmacon Research (Lafayette, Colo., USA), Pierce Chemical (Rockford, Ill., USA), ChemGenes (Ashland, Mass., USA), Proligo (Hamburg, Germany), and Cruachem (Glasgow, UK).

Inventive siRNAs may be comprised entirely of natural RNA nucleotides, or may instead include one or more nucleotide analogs and/or modifications as mentioned above for antisense molecules. The siRNA structure may be stabilized, for example by including nucleotide analogs at one or more free strand ends in order to reduce digestion, e.g., by exonucleases. Alternatively, siRNA molecules may be generated by in vitro transcription of DNA sequences encoding the relevant molecule. Such DNA sequences may be incorporated into a wide variety of vectors with suitable RNA polymerase promoters such as T7, T3, or SP6.

Antibodies

In some embodiments, antibodies can be screened for potential synaptic vesicle cycling modulators. For example, antibodies may be designed based on sequence information of proteins and genes involved in synaptic vesicle cycling. For example, antibodies can be designed to target presynaptic proteins including, but not limited to, intrinsic vesicle proteins (e.g., synaptic vesicle protein 2 (SV2), synaptophysins, synaptotagmins, vesicle-associated membrane polypeptides (VAMPs), neurotransmitter transporters (NT transporters), synaptogyrins, proton pump), peripheral vesicle proteins (e.g., Rabs, cystine string proteins (CSPs), synapsins), synaptic plasma membrane proteins (e.g., calcium channels, synaptosome-associated protein of 25 kDa (SNAP-25), syntaxin), and cytosolic proteins (e.g., SNAPs, n-Sec1), synapsins (e.g., synapsin I, II and III), dephosphins (e.g., dynamin I, PIP5K1γ, and synaptojanin I). Antibodies can also be designed to target calcineurin, calcineurin activators, inhibitors, and/or substrates.

Antibodies can be generated using methods well known in the art. For example, protocols for antibody production are described by Harlow and Lane, Antibodies: A Laboratory Manual, (1988). Typically, antibodies can be generated in mouse, rat, guinea pig, hamster, camel, llama, shark, or other appropriate hosts. Alternatively, antibodies may be made in chickens, producing IgY molecules (Schade et al., (1996) ALTEX 13(5):80-85). In some embodiments, antibodies suitable for the present invention are subhuman primate antibodies. For example, general techniques for raising therapeutically useful antibodies in baboons may be found, for example, in Goldenberg et al., international patent publication No. WO 91/11465 (1991), and in Losman et al., Int. J. Cancer 46: 310 (1990). In some embodiments, monoclonal antibodies may be prepared using hybridoma methods (Milstein and Cuello, (1983) Nature 305(5934):537-40.). In some embodiments, monoclonal antibodies may also be made by recombinant methods (U.S. Pat. No. 4,166,452, 1979).

In some embodiments, antibodies suitable for the invention may include humanized or human antibodies. Humanized forms of non-human antibodies are chimeric Igs, Ig chains or fragments (such as Fv, Fab, Fab′, F(ab′)2 or other antigen-binding subsequences of Abs) that contain minimal sequence derived from non-human Ig. Generally, a humanized antibody has one or more amino acid residues introduced from a non-human source. These non-human amino acid residues are often referred to as “import” residues, which are typically taken from an “import” variable domain. Humanization is accomplished by substituting rodent CDRs or CDR sequences for the corresponding sequences of a human antibody (Riechmann et al., Nature 332(6162):323-7, 1988; Verhoeyen et al., Science. 239(4847):1534-6, 1988.). Such “humanized” antibodies are chimeric Abs (U.S. Pat. No. 4,816,567, 1989), wherein substantially less than an intact human variable domain has been substituted by the corresponding sequence from a non-human species. In some embodiments, humanized antibodies are typically human antibodies in which some CDR residues and possibly some FR residues are substituted by residues from analogous sites in rodent Abs. Humanized antibodies include human Igs (recipient antibody) in which residues from a complementary determining region (CDR) of the recipient are replaced by residues from a CDR of a non-human species (donor antibody) such as mouse, rat or rabbit, having the desired specificity, affinity and capacity. In some instances, corresponding non-human residues replace Fv framework residues of the human Ig. Humanized antibodies may comprise residues that are found neither in the recipient antibody nor in the imported CDR or framework sequences. In general, the humanized antibody comprises substantially all of at least one, and typically two, variable domains, in which most if not all of the CDR regions correspond to those of a non-human Ig and most if not all of the FR regions are those of a human Ig consensus sequence. The humanized antibody optimally also comprises at least a portion of an Ig constant region (Fc), typically that of a human Ig (Riechmann et al., Nature 332(6162):323-7, 1988; Verhoeyen et al., Science. 239(4847):1534-6, 1988.).

Human antibodies can also be produced using various techniques, including phage display libraries (Hoogenboom et al., Mol Immunol. (1991) 28(9):1027-37; Marks et al., J Mol Biol. (1991) 222(3):581-97) and the preparation of human monoclonal antibodies (Reisfeld and Sell, 1985,Cancer Surv. 4(1):271-90). Similarly, introducing human Ig genes into transgenic animals in which the endogenous Ig genes have been partially or completely inactivated can be exploited to synthesize human antibodies. Upon challenge, human antibody production is observed, which closely resembles that seen in humans in all respects, including gene rearrangement, assembly, and antibody repertoire (Fishwild et al., High-avidity human IgG kappa monoclonal antibodies from a novel strain of minilocus transgenic mice, Nat Biotechnol. 1996 July; 14(7):845-51; Lonberg et al., Antigen-specific human antibodies from mice comprising four distinct genetic modifications, Nature 1994 April 28; 368(6474):856-9; Lonberg and Huszar, Human antibodies from transgenic mice, Int. Rev. Immunol. 1995; 13(1):65-93; Marks et al., By-passing immunization: building high affinity human antibodies by chain shuffling. Biotechnology (N Y). 1992 July; 10(7):779-83).

Others

In some embodiments, natural products libraries can be screened using assays of the invention.

In some embodiments, neuronal cells may be incubated with test agents for a selected time prior to subjecting the cells to stimulation (e.g., electrical stimulation). In various embodiments, selected amounts of test agent are added to wells comprising neuronal cells. In various embodiments, differing amounts and/or types of test agent are added to different wells comprising neuronal cells. In various embodiments, substantially identical amounts and/or types of test agent are added to different wells comprising neuronal cells.

In many embodiments, the assay results with test agents can be compared to a control in order to determine if any member agents modulate one or more activities of synaptic vesicle cycling. In some embodiments, a control is the same one or more activities of synaptic vesicle cycling detected in an otherwise identical neuronal cell that has not been treated with testing agents. In some embodiments, a neuronal cell may exhibit normal synaptic vesicle cycling without being treated with test agents. In some embodiments, an untreated neuronal cells may exhibit defective synaptic vesicle cycling (e.g., neuronal cells isolated from an animal or a human being suffering from a neuronal disorder). In some embodiments, a test agent is considered a potential synaptic vesicle cycling enhancer if it causes enhancement of synaptic vesicle cycling relative to untreated controls. In some embodiments, a test agent is considered a potential synaptic vesicle cycling inhibitor if it causes inhibition of synaptic vesicle cycling relative to untreated controls.

In some embodiments, specific activities of synaptic vesicle cycling can be measured using methods in accordance with the present invention. In particular, activities in various phases of synaptic vesicle cycling (e.g., exocytosis, endocytosis, recycling, etc.) can be measured. In some embodiments, exocytosis can be measured using parameters including, but not limited to, release probability, release trains, docked vesicles, pool size. In some embodiments, endocytosis can be measured using parameters indicating, for example, non-saturating, saturating, or steady state. As used herein, non-saturating indicates endocytosis when the vesicle internalization sites are non-limiting. Saturating indicates endocytosis when the vesicle internalization sites are limiting. In some embodiments, recycling can be measured by measuring response size after restimulation after a rest period (e.g., 1 minute) relative to first response. In some embodiments, high frequency firing can be measured. As used herein, the term “high frequency firing” typically refers to firing at frequencies at or above 10 Hz. Typically, the period of sustained high frequency firing that results in synaptic vesicle release is inversely proportional to the frequency of firing. Under certain conditions, synaptic fatigue may occur during high frequency firing. For example, it is contemplated that the size of the readily releasable pool of synaptic vesicles is limited (e.g., 10 vesicles). Therefore, at firing frequencies at or above 10 Hz, synaptic fatigue occurs when firing persists beyond the point when the readily releasable pool is expended, e.g., beyond more than 10 action potentials. High frequency firing can be measured using electrophysiology (e.g., extracellular field recording) or imaging analysis (e.g., using synaptopHluorins).

Prospective modulators can be further tested to confirm the modulatory effects and/or to predict efficacy using suitable synaptic vesicle cycling assays described herein. Prospective modulators can also be tested using other assays to confirm their effects on synaptic vesicle cycling or neurotransmission signaling pathways. In some embodiments, prospective modulators can be tested using phosphosignature assays to determine their ability to modulate calcineurin and/or phosphorylation status of dephosphins or other synaptic proteins. In some embodiments, prospective modulators can be further tested for their ability to treat neurological or psychiatric disorders, in particular, those associated with synaptic vesicle cycling dysfunction. In some embodiments, prospective modulators can be tested in animal models. For example, prospective modulators can be tested in animal models of neurological or psychiatric disorders (e.g., mouse models of schizophrenia). In some embodiments, prospective modulators can be tested in electrophysiology assays correlative with synaptic vesicle cycling (e.g., synaptic fatigue, recovery from synaptic fatigue.

Synaptic vesicle cycling modulators identified in accordance with the present invention may be involved in regulating various steps, processes and/or biological pathways directly or indirectly involved in synaptic vesicle cycling. For example, calcineurin activation (e.g., by direct activation or inhibition of calcineurin's interaction with an inhibitor (i.e. DSCR1 or cabin) or inhibition of an enzymatic inhibition) may enhance mobilization and endocytosis. Cyclin-dependent kinase 5 (CDK5) inhibition may enhance synaptic vesicle cycling. Dual-specificity tyrosine-phosphorylated and regulated kinase 1A (Dyrk1A) or protein kinase C (PKC) inhibition may also enhance synaptic vesicle endocytosis. Inhibition of extracellular signal-regulated kinases (ERKs) (e.g., ERK1) may enhance synaptic vesicle endocytosis and/or mobilization. Modulation of Ca⁺⁺ channels, including modulation of Ca⁺⁺ effluxers or other proteins that remove or sequester Ca⁺⁺, may modulate any part of the process. G-protein coupled receptors (GPCRs) (including muscarinic acetylcholine receptors) modulate all steps of synaptic vesicle cycling; therefore, agonists and antagonists of these receptors may be identified as modulators of various aspects of synaptic vesicle cycling. Exemplary aspects of synaptic vesicle cycling that can be affected by modulators and the assay parameters to identify those modulators are shown in Table 1.

### Therapeutic Applications

Among other things, the present invention encompasses the recognition that substances that modulate synaptic vesicle cycling may be useful in the treatment of certain disorders (e.g., disorders associated with synaptic vesicle dysfunction). In some embodiments, synaptic vesicle cycling modulators identified herein can be used to treat (e.g., alleviate, ameliorate, relieve, inhibit, prevent, delay onset of, reduce severity of, and/or reduce incidence of) one or more symptoms or features of a disease, disorder, and/or condition associated with synaptic vesicle cycling dysfunction. For example, the present invention encompasses the recognition that modulators of synaptic vesicle cycling may be useful in the treatment of psychiatric disorders, diseases, or conditions including, but not limited to, schizophrenia, bipolar disorder, epilepsy; neurological diseases, disorders or conditions including, but not limited to, Alzheimer's disease, alcoholic Korsakoff disease (KS), multiple sclerosis, and Parkinson's disease; and/or other diseases, disorders or conditions including, but not limited to, Down's syndrome, Williams syndrome, Specific Language Impairment, and Attention Deficit Hyperactivity Disorder (ADHD).

As non-limiting examples, modulators that inhibit synaptic vesicle cycling (e.g., inhibition of excitatory synaptic transmission, or enhancement of inhibitory synaptic vesicle cycling) may be used to treat epilepsy. Modulators that regulate (e.g., enhance or stabilize) serotonergic synaptic vesicle cycling may be used to treat depression. Modulators that regulate (e.g., enhance or stabilize) dopaminergic vesicle cycling may be used to treat Parkinson's diseases. Modulators that regulate (e.g., enhance) cholinergic vesicle cycling may be used to treat Alzheimer disease. Modulators that regulate (e.g., enhance or stabilize) glutaminergic synaptic vesicle cycling may be used to treat schizophrenia. Modulators that regulate (e.g., reducer or stabilize) adrenergic or noradrenergic vesicle cycling may be used to treat anxiety disorders.

Synaptic vesicle cycling modulators and/or pharmaceutical compositions thereof may be administered to a subject using any amount effective for treating a disease, disorder, and/or condition (e.g., a disease, disorder, and/or condition relating to synaptic vesicle cycling dysfunction). The specific therapeutically effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In certain embodiments, synaptic vesicle cycling modulators and/or compositions thereof may be administered at dosage levels sufficient to deliver from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations).

### Pharmaceutical Compositions

The present invention further provides pharmaceutical compositions comprising one or more synaptic vesicle cycling modulators, together with one or more pharmaceutically acceptable excipients. Such pharmaceutical compositions may optionally comprise one or more additional therapeutically-active substances.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, shaping and/or packaging the product into a desired single- or multi-dose unit.

A pharmaceutical composition in accordance with the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered. By way of example, the composition may comprise between 0.1% and 100% (w/w) active ingredient.

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21^{st} Edition, A. R. Gennaro (Lippincott, Williams & Wilkins, Baltimore, Md., 2006; incorporated herein by reference) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical formulations. Excipients such as cocoa butter and suppository waxes, coloring agents, coating agents, sweetening, flavoring, and/or perfuming agents can be present in the composition, according to the judgment of the formulator.

General considerations in the formulation and/or manufacture of pharmaceutical agents may be found, for example, in Remington: The Science and Practice of Pharmacy 21^{st} ed., Lippincott Williams & Wilkins, 2005 (incorporated herein by reference).

### Administration

Synaptic vesicle cycling modulators and/or pharmaceutical compositions thereof in accordance with the present invention may be administered by any route. In some embodiments, synaptic vesicle cycling modulators and/or pharmaceutical compositions thereof are administered by one or more of a variety of routes, including oral, intravenous, intramuscular, intra-arterial, intramedullary, intrathecal, subcutaneous, intraventricular, transdermal, interdermal, rectal, intravaginal, intraperitoneal, topical (e.g. by powders, ointments, creams, gels, lotions, and/or drops), mucosal, nasal, buccal, enteral, vitreal, intratumoral, sublingual; by intratracheal instillation, bronchial instillation, and/or inhalation; as an oral spray, nasal spray, and/or aerosol, and/or through a portal vein catheter. In some embodiments, synaptic vesicle cycle modulators and/or pharmaceutical compositions thereof are administered by systemic intravenous injection. In specific embodiments, synaptic vesicle cycling modulators and/or pharmaceutical compositions thereof may be administered intravenously and/or orally. In specific embodiments, synaptic vesicle cycling modulators and/or pharmaceutical compositions thereof may be administered in a way which allows the synaptic vesicle cycling modulators to cross the blood-brain barrier. Many strategies are available for crossing the blood-brain barrier, including but not limited to, increasing the hydrophobic nature of a molecule; introducing the molecule as a conjugate to a carrier, such as transferrin, targeted to a receptor in the blood-brain barrier; and the like. In another embodiment, the molecule can be administered intracranially or, more preferably, intraventricularly. In another embodiment, osmotic disruption of the blood-brain barrier can be used to effect delivery of an active agent to the brain (Neuwelt et al., 1995, Proc. Natl. Acad. Sci. USA “Delivery of herpesvirus and adenovirus to nude rat intracerebral tumors after osmotic blood-brain barrier disruption,” Oct. 10, 1995). In yet another embodiment, an agent can be administered in a liposome targeted to the blood-brain barrier. Administration of pharmaceutical agents in liposomes is known (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.). All of such methods are envisioned in the present invention. In specific embodiments, synaptic vesicle cycling modulators and/or pharmaceutical compositions thereof may be administered by portal vein catheter. However, the invention encompasses the delivery of synaptic vesicle cycling modulators and/or pharmaceutical compositions thereof by any appropriate route taking into consideration likely advances in the sciences of drug delivery.

In general the most appropriate route of administration will depend upon a variety of factors including the nature of synaptic vesicle cycling modulators (e.g., their stability in the environment of the gastrointestinal tract, bloodstream, etc.), the condition of the patient (e.g., whether the patient is able to tolerate particular routes of administration), etc. The invention encompasses the delivery of the pharmaceutical compositions by any appropriate route taking into consideration likely advances in the sciences of drug delivery.

### Kits

The invention provides a variety of kits for conveniently and/or effectively carrying out methods of the present invention. Typically kits will comprise sufficient amounts and/or numbers of components to allow a user to perform multiple treatments of a subject(s) and/or to perform multiple experiments.

In some embodiments, kits comprise one or more of (i) a reporter for tracking synaptic vesicle cycling; (ii) a plurality of wells suitable for culturing a plurality of neuronal cells; (iii) a stimulation system; (iv) a detection system; and (v) one or more test substances. In some embodiments, kits may further optionally comprise one or more of (vi) a positive control substance known to modulate synaptic vesicle cycling; (vii) a negative control substance known not to modulate synaptic vesicle cycling; and (viii) instructions for use.

In some embodiments, kits comprise one or more synaptic vesicle cycling modulators identified using synaptic vesicle cycling assays, as described herein. In some embodiments, such a kit is used in the treatment, diagnosis, and/or prophylaxis of a subject suffering from and/or susceptible to a disease, condition, and/or disorder (e.g. associated with synaptic vesicle cycling dysfunction). In some embodiments, such a kit comprises one or more of (i) at least one synaptic vesicle cycling modulator or pharmaceutical composition thereof; (ii) a syringe, needle, applicator, etc. for administration of the to a subject; and (iii) instructions for use.

In some embodiments, kits comprise a collection of different reporters; cell types; controls; test substances; synaptic vesicle cycling modulators, and/or pharmaceutical compositions thereof; etc.

In some embodiments, kits may include additional components or reagents. For example, kits may comprise cell culture media, tissue culture media, buffers, etc. In some embodiments, kits may comprise instructions for use. For example, instructions may inform the user of how to perform a screen to identify synaptic vesicle cycling modulators. Instructions may inform the user of the proper procedure by which to prepare a pharmaceutical composition comprising modulators and/or the proper procedure for administering a pharmaceutical composition to a subject.

In some embodiments, kits include a number of unit dosages of a pharmaceutical composition comprising synaptic vesicle cycling modulators. A memory aid may be provided, for example in the form of numbers, letters, and/or other markings and/or with a calendar insert, designating the days/times in the treatment schedule in which dosages can be administered. Placebo dosages, and/or calcium dietary supplements, either in a form similar to or distinct from the dosages of the pharmaceutical compositions, may be included to provide a kit in which a dosage is taken every day.

Kits may comprise one or more vessels or containers so that certain of the individual components or reagents may be separately housed. Kits may comprise a means for enclosing individual containers in relatively close confinement for commercial sale (e.g., a plastic box in which instructions, packaging materials such as styrofoam, etc., may be enclosed).

### EXEMPLIFICATION

### Example 1: pHluorin Reporter

In this example, synaptic vesicle cycling was studied using a pH-sensitive GFP, termed pHluorin, that was tagged to the intra-lumenal domain of synaptic vesicle proteins. Vesicle proteins that were used in this fashion include VAMP, synaptotagmin, vGlut1, and synaptophysin. When synapses were at rest, these proteins placed pHluorin in the synaptic vesicle lumen, which maintained an internal pH of 5.5. At this acidic pH, fluorescence was quenched. When stimulated, the synaptic vesicle was exocytosed and the protons were secreted, causing the pH to rise, and the pHluorin to fluoresce. Vesicles were then reinternalized and reacidified, again quenching fluorescence. In this way, fluorescence changes measured at a single synapse were used to track the synaptic vesicle cycle. In this example, synaptopHluorin (i.e., a fusion of the vesicle protein VAMP2 and of a pH-sensitive GFP fused to vGlut1) was used.

By introduction into a high percentage of neurons plated at high density within a well, synaptopHluorins were used to measure synaptic vesicle cycling at the whole-well, population level. One way synaptopHluorin was introduced into neurons was by viral infection. Viral delivery of synaptopHluorin was used to validate the ability to measure the synaptic vesicle cycle at the population level. Adeno-associated virus (MOI=1000) was used to deliver synaptopHluorin into rat embryonic cortical neurons plated in 96-well optically-clear plastic-bottomed plates (Nunc) at 11 days in vitro. At 21 days in vitro, neurons were imaged on a Zeiss Axiovert Z1 inverted epifluoresence microscope using low magnification optics (10×, 0.45 NA objective) insufficient to resolve individual synapses. Neurons in the presence or absence of external Ca⁺⁺ were treated with ionomycin, a calcium ionophore that triggers synaptic vesicle exocytosis and the resulting fluorescence of synaptopHluorin. Cultures treated with ionomycin in the presence of external Ca⁺⁺ showed an approximately 20% increase in synaptopHluorin signal, whereas the response of cultures in the absence of external Ca⁺⁺ was significantly attenuated (about 3%). Thus, this example demonstrates that the synaptic vesicle cycle can be measured at the population level with low magnification optics using synaptopHluorin as a reporter.

Alternatively or additionally, pHluorin-based synaptic vesicle cycle reporters are introduced into a large percentage of cultured neurons by generating transgenic animals (e.g., mice or rats) that express a reporter (e.g., synaptophysin-pHluorin) in the brain. Any method known by those of skill in the art can be used to generate such transgenic animals.

### Example 2. pH-Sensitive Dye Reporters

A second exemplary reporter for measuring the synaptic vesicle cycle utilizes the acidic intravesicular pH and acid sensing dyes. Endocytosis reporters are commercially available that increase their fluorescence when the pH is raised from the extracellular pH of 7.4 to the intravesicular pH of 5.5. These dyes include CypHer5E (GE Healthcare) and pHrodo (Invitrogen). These dyes function as reporters of synaptic vesicle cycling by entering synaptic vesicles when the neurons are stimulated. When vesicles are endocytosed and reacidified, the reporter fluoresces. In this way, endocytosis is measured by an increase in fluorescence intensity, and ensuing exocytosis is measured by a decrease in fluorescence intensity.

pH-sensitive dyes are delivered to synaptic vesicles using any method. pH-sensitive dyes are conjugated directly to an antibody that recognizes the intralumenal domain of a synaptic vesicle protein (e.g., a monoclonal antibody that recognizes the intralumenal domain of synaptotagmin; Synaptic Systems). Alternatively or additionally, dyes are conjugated to a membrane intercalating substance (e.g., a styryl dye, such as FM1-43). When pH-sensitive dyes are utilized alone, when neurons are stimulated, synaptic vesicles exocytose, allowing dye to enter synaptic vesicles. Synaptic vesicles are then endocytosed, and the remaining surface dye is washed away, often by several wash steps. By conjugating pH-sensitive dyes to the intercalating component of, e.g., FM1-43 dye, a reporter for synaptic vesicle cycling is generated that tracks synaptic vesicle cycling without the washing steps.

In order to utilize dyes, cells are typically incubated in an assay buffer. pH-sensitive dye (e.g., conjugated to an antibody, membrane intercalater, etc.) is added to the assay buffer. Cells are stimulated with a series of electrical voltages, causing synaptic vesicle exocytosis and entry of dye into synaptic vesicles. When synaptic vesicles endocytose, dye is trapped inside the reformed vesicle. When synaptic vesicles reacidify, the dye fluoresces when excited by the proper wavelength of light.

### Example 3. Imaging Systems

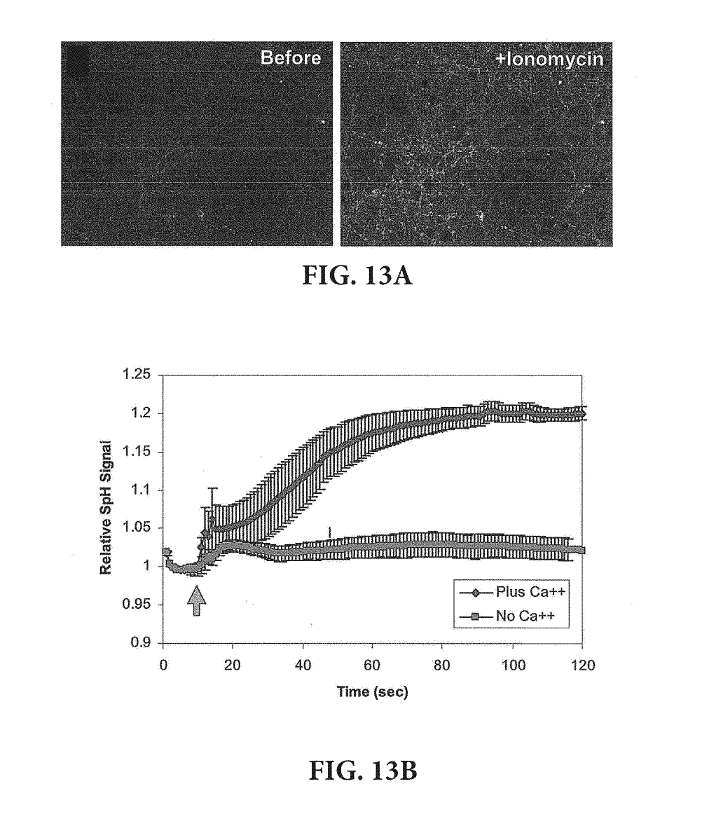

A Zeiss Axiovert Z1 with a 10×, 0.45 NA objective lens was used to image the synaptic vesicle cycle at the whole well level in primary neurons infected with an adeno-associated virus that expresses synaptopHluorin (see FIG. 13). In addition, there was open access to the wells of the plate using this microscope to allow access of a stimulation system. The stimulation system was used to detect the effects of individual compounds on synaptic vesicle cycling. The process of using an epifluorescence microscope to image synaptic vesicle cycling typically took about 2 minutes and, thus, about 2-3 hours was typically required to image all wells of a 96-well plate in this configuration.

An alternative imaging system, which was used, is the commercially available plate reader plate::vision (Perkin Elmer). This plate reader was capable of kinetic-based fluorescence analysis, allowed access to the top of the plate for the integration of a stimulation system, had high collection efficiency optics, and had special optics designed for the analysis of 96 wells in parallel.

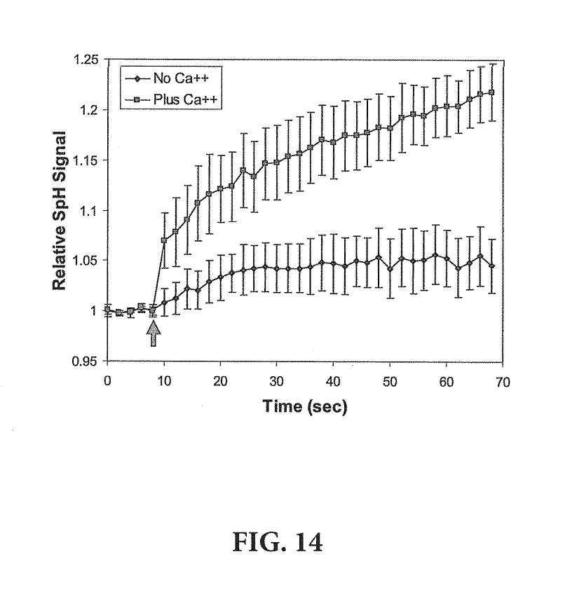

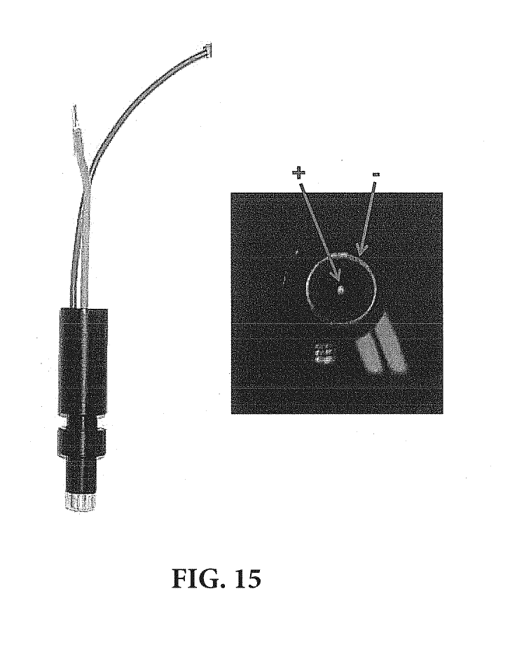

The plate::vision plate reader was evaluated for its ability to record the synaptic vesicle cycle in multiple wells in parallel. Primary rat neuronal cultures in 96-well plates were infected with the synaptopHluorin adeno-associated virus at 12 days in vitro. Culture medium was replaced with Tyrode's solution plus or minus external Ca⁺⁺. A baseline recording of fluorescence was measured in the entire plate on the plate::vision plate reader. Then, ionomycin was added to all wells, and fluorescence changes were measured every 2 seconds for 1 minute. Results showed that there was about 20% increase in fluorescence in wells stimulated with ionomycin in the presence of Ca⁺⁺, whereas the absence of Ca⁺⁺ largely abolished the fluorescence increase (<5%). These changes were similar to those observed when a similar experiment was performed on the Zeiss inverted epifluorescence microscope. These data show that the plate::vision plate reader measured the synaptic vesicle cycle in a 96 well plate in multiple wells in parallel (FIG. 14).

### Example 4. Electrical Stimulation System

In order to visualize the synaptic vesicle cycle, neurons were stimulated to induce exocytosis and endocytosis of synaptic vesicles. Primary neurons in culture were stimulated by the application of an electrical field potential, in part, as described in Ryan and Smith (1995, Neuron, 14:983; incorporated herein by reference) and in Ryan et al. (1996, Proc. Natl. Acad. Sci., 93:5567; incorporated herein by reference). In these examples, field potential was applied to primary neurons in a 96 well format. A 96 well electrical stimulator was custom-developed and used to generate time-varying electric fields in the wells comprising neuronals. The ability of the electrode designs to trigger the synaptic vesicle cycle was measured on a detection apparatus, which was either a Zeiss Axiovert Z1 inverted epifluorescence microscope or the plate::vision plate reader, as described above.

Custom-Developed 96 Well Electrical Stimulator

In this example, a custom electrode pair was used to stimulate synaptic vesicle cycling. The custom electrode pair had an outer platinum ring electrode with an outer diameter of about 6 mm, which fit into the well of a 96 well plate. In the center of the well a 1 mm diameter gold wire was placed (FIG. 6C). The gold center wire served as the cathode and the outer platinum ring as the anode, and they were held in place by a plastic apparatus. The electrode was placed into the well of a 96-well plate and was connected to a stimulus isolator which was connected to a pulse generator. In order to stimulate 96 wells in parallel, 96 of these electrode pairs are connected into an 8×12 array.

Custom Modification of Multi-Well Plate Electroporation System

The CellAxessHT (available from Cellectricon AB, Gothenburg, Sweden) is a high-throughput electroporator designed to deliver siRNAs into cells in 384 plates. It has a 96 well electroporation head, with each electrode pair designed to fit into a well of a 384 well plate. To serve as the stimulation component of the synaptic vesicle cycling assay in this example, the electrodes of this system were modified and/or optimized to maximize the electric field potential coverage of the wells of 96-well plates.

### Example 5. Customized Cellectricon CX3 Electrode System

In this example, the stimulation of action potential firing was demonstrated with a modified, commercially-available, electroporation system. A custom-modified CellAxess CX3 electroporation system, available from Cellectricon AB was obtained for the experiment. This system provided three pairs of electrodes which could substantially simultaneously electrically stimulate three separate neuronal cell cultures within three wells of a 96-well plate. The instrument's electrodes were modified to provide substantially uniform electric fields and/or current density within the electrode's excitation regions. Additionally, the plastic components used to mount the electrodes was modified to comprise low-fluorescence material, which minimized the background fluorescence during data acquisition.

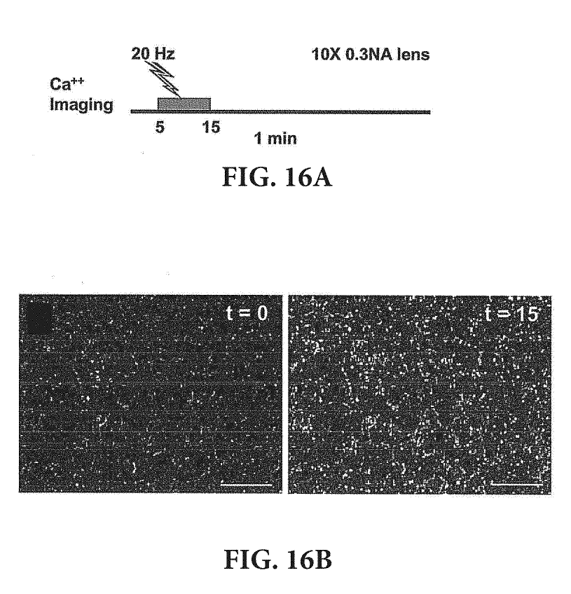

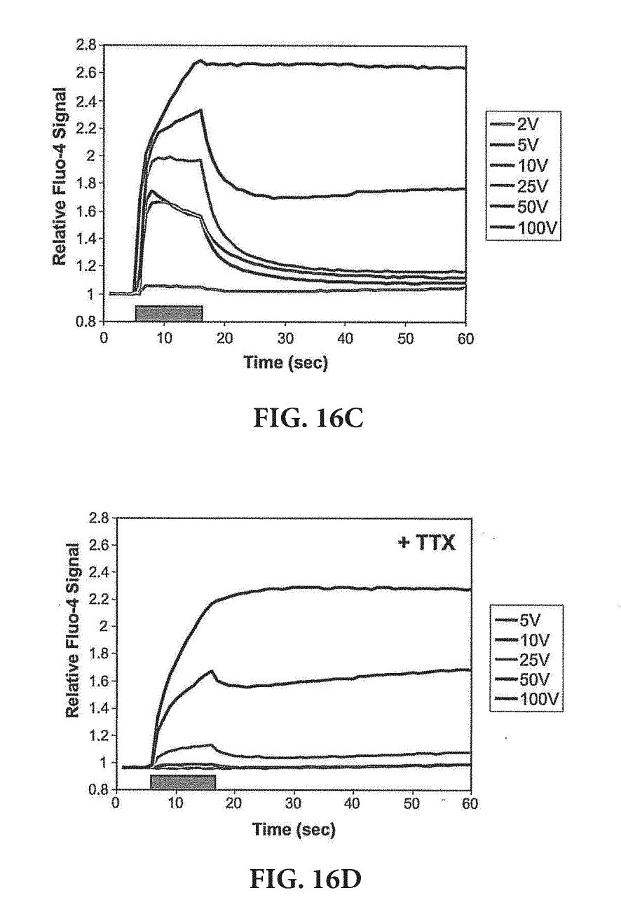

The customized CX3 was tested for its ability to induce action potential in neuronal cells cultivated in 96-well plates. For this example, neurons were loaded with a Fluo-4 Ca⁺⁺ indicator and stimulated using the CX3 system. Electrical stimulation was carried out at 20 Hz for 10 seconds while time-lapsed images were acquired using a Zeiss Axiovert Z1 with a 10×, 0.3 numerical aperture objective lens. Stimulation voltages were varied between about 2 volts and about 100 volts. Certain experimental conditions are depicted in FIG. 16A. The lower line indicates the duration of imaging, with the Axiovert Z1 system, of a region of a well comprising a plurality of neuronal cells. The imaging period was about 1 minute. The block indicates the excitation period.

When electrical stimulation was carried out at 10V, a large increase in Ca⁺⁺ influx due to the electrical field stimulation was observed in acquired images of the neuronal cells. FIG. 16B reports the increase in Ca⁺⁺ influx. The image acquired at t=0 seconds shows the baseline fluorescence emission from the neuronal cell before the application of electrical stimulation. The image acquired at t=15 seconds shows increased fluorescence emission at the end of the excitation period. The increased fluorescent emission results from an increase in Ca⁺⁺ influx within the neurons and indicates action potential firing.

Several trials were then carried out over a range of voltage values to study kinetic fluorescent behavior within the wells comprising neuronal cells. Traces of relative Fluo-4 signal recorded for the different excitation voltages are plotted as a function of time in FIG. 16C. The results show the existence of a broad range of excitation voltages, between 5 volts and about 25 volts, that triggered strong Ca⁺⁺ influx and was followed by a return to baseline after termination of the electrical stimulation. At higher voltages, greater than about 50 volts, the Ca⁺⁺ signal did not return to baseline, indicating toxicity at these high voltages.

In an additional test of the biodynamics of the electrically-stimulated neuronal cells, TTX was added to several wells systems which were then subjected to the same electrical stimulation reported in FIG. 16C. The results for this test are shown in FIG. 16D. The addition of TTX, a voltage-gated sodium channel blocker, blocked the neurons' responses at the low voltages, indicating that the Ca⁺⁺ flux at these frequencies is due to the generation of action potentials by the CX3 electrode stimulation.

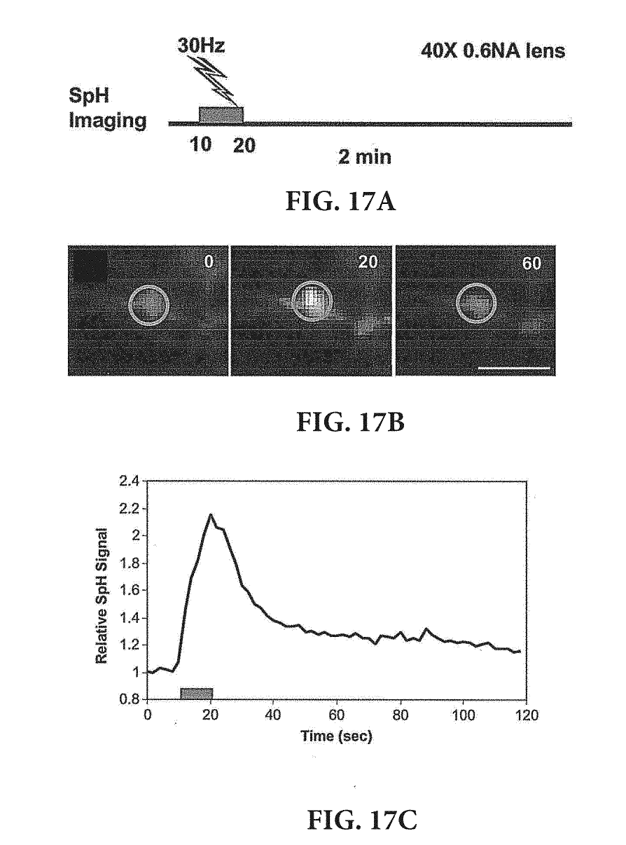

The customized CX3 electrode system was tested for its ability to induce synaptic vesicle cycling in neurons in 96-well plates. Neurons were infected with an adeno-associated virus that expresses synaptopHluorin. Cells were stimulated at about 30 Hz and about 10 volts with the CX3 electrode system while time-lapse images were obtained with the Zeiss Axiovert Z1 using a 40×, 0.6 numerical aperture air objective lens. Certain experimental conditions are reported in FIG. 17A. The imaging period was about 120 seconds, and the electrical stimulation period was about 10 seconds. An increase in fluorescence followed by a return to baseline was observed for individual synapses, as shown in FIG. 17B. The peak fluorescence occurred at about the end of the electrical stimulation period, after which the fluorescent signal returned to about baseline. FIG. 17C shows the densitometry measurement of the synapse depicted in FIG. 17B, and reports the kinetic fluorescent behavior for the synapse. These data show that the CX3 electrode can induce synaptic vesicle cycling in neuronal synapses 96-well plates.

### Example 6. Technology Integration

Selected stimulation and detection systems can be integrated into a single synaptic vesicle screening system. For example, a system has been developed to measure synaptic vesicle cycling in 96 wells in parallel. Primary neurons in 96-well plates have been infected on the 12^{th} day in vitro with the synaptophysin-pHluorin-expressing adeno-associated virus. On the 21^{th} day in vitro, culturing media was replaced with assay buffer, and electrodes were placed into the wells. Time-varying electric fields were applied to neuronal cells at 10V/10 Hz for 30 seconds. During this time, fluorescence, e.g., generated by synaptopHluorin, was recorded by the detection apparatus.

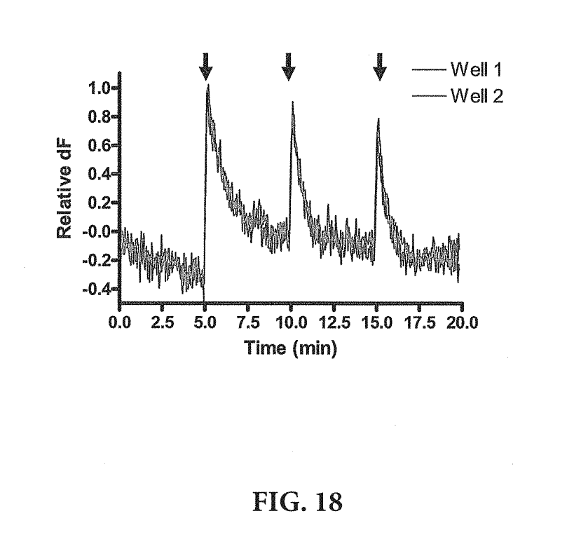

In this particular example, we have demonstrated that the plate::vision plate reader, the customized Cellectricon CX3 electrode, and synaptophysin-pHluorin reporter can be successfully integrated. Rat primary cortical neurons in 96-well plates were infected with a synaptophysin-pHluorin AAV (MOI 2000) at 7 DIV. At 30 DIV, cultures were loaded onto a plate::vision plate reader and the Cellectricon CX3 custom electrode was placed into two of these wells. A kinetic fluorescence read was performed at 2 seconds/image for 20 minutes, and the neurons were stimulated with a 30 Hz, 20V pulse train for 10 seconds. This stimulation was applied 3 times at 5 minute intervals (red arrows). FIG. 18 shows the results from these wells. The change in fluorescence (dF) was normalized to the peak fluorescence reading within each well. These data show that the plate::vision plate reader can measure the synaptic vesicle cycle using the synaptophysin-pHluorin reporter when neurons are stimulated with the customized Cellectricon electrode.

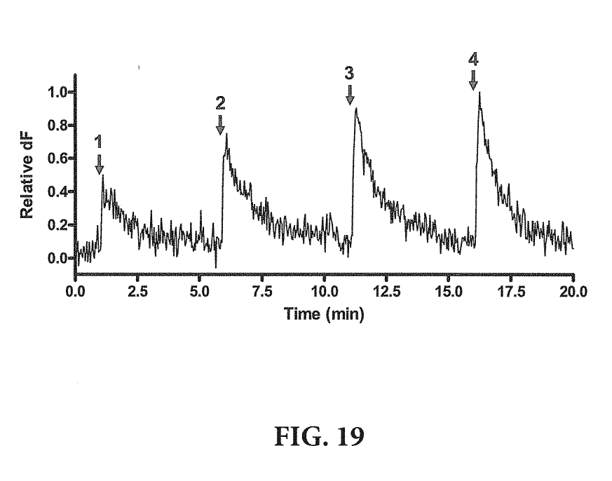

We further established that this system can successfully detect the synaptophysin-pHluorin response to different numbers of stimulated action potentials. Rat primary cortical neurons were cultured in 96-well plates and infected with a synaptophysin-pHluorin AAV (MOI 2000) at 7 DIV as described above. At 30 DIV, cultures were loaded onto a plate::vision plate reader, and the Cellectricon CX3 custom electrode was placed into two of these wells. A kinetic fluorescence read was performed at 2 seconds/image for 20 minutes, and the neurons were stimulated with a 30 Hz, 20V pulse train for 50 (Arrow 1), 100 (Arrow 2), 200 (Arrow 3), or 300 (Arrow 4) stimuli (see FIG. 19). The change in fluorescence (dF) was normalized to the peak fluorescence reading within each well. These data show the sensitivity of the plate::vision plate reader and its capabilities for measuring a small number of action potentials. It is contemplated that, due to the high signal-to-noise ratio of the plate::vision system and the summation of the action potential responses during stimulation trains, that the plate::vision is capable of detecting the synaptophysin-pHluorin response to 50 or fewer action potentials delivered at 10 Hz or lower.

### Example 7. Measuring Various Parameters of Synaptic Vesicle Cycling

Various parameters of synaptic vesicle cycling are measured by altering the stimulation patterns. Exemplary parameters and stimulation paradigms are provided in Table 1.

TABLE 1

| | | | | | pHluroin |
|---|---|---|---|---|---|
| Phase | Parameter | Stimulus | Duration | | Measurement |
| Exocytosis | Release | .05 Hz | >10 | AP | Presence of |
| | Probability | | | | vesicle release |
| | Release Trains | >10 Hz | >10 | AP | Rising phase |
| | Docked | 100 Hz | 1 | sec | Rising phase |
| | Vesicles | | | | |
| | Pool Size | 30 Hz | 20 | sec | Steady state |
| | | | | | level |
| Endocytosis | Non-Saturating | 10 Hz | <10 | sec | Falling phase |
| | Saturating | 10 Hz | >10 | sec | Falling phase |
| | Steady State | 10 Hz | >30 | sec | Steady state |
| | | | | | level |
| Recycling | Recycling | 10 Hz | 200 | AP | Response size |

| | |
|---|---|
| Re- | relative to |
| stimulation | first response |
| after | |
| 1 minute | |

AP: Action potential
Non-saturating indicates endocytosis when the vesicle internalization sites are non-limiting.
Saturating indicates endocytosis when the vesicle internalization sites are limiting.
The terms “rising phase,” “steady state level,” and “falling phase” are used herein with meanings as understood by those skilled in the art.

### Example 8. High-Content Screening System

A high-content screening system was developed using an alternative imaging system, the BD pathway high content imaging system. Such high-content screening systems are typically custom-built or commercially available from, e.g., Pathway, Becton Dickinson, Franklin Lakes, N.J.; ImageXpress MICRO (Molecular Devices, Union City, Calif.; Opera, Perkin Elmer, Waltham, Mass.; or ArrayScan, Thermo Fisher Scientific, Waltham, Mass. These systems have high optical sensitivity and generate high resolution time-lapse image stacks during kinetic assays. As a result, the data have single synapse resolution and high signal-to-noise ratios. However, these systems typically only record individual wells of multiwell plates sequentially. Therefore, since it typically takes about two minutes to image the synaptic vesicle cycle, it takes about two to three hours to image all wells of a 96-well plate. Thus, a high-content screening system is typically able to measure at least about 20 (e.g., 25, 30, 35, 40, 45, or 50) synaptic vesicle cycling assays per hour.

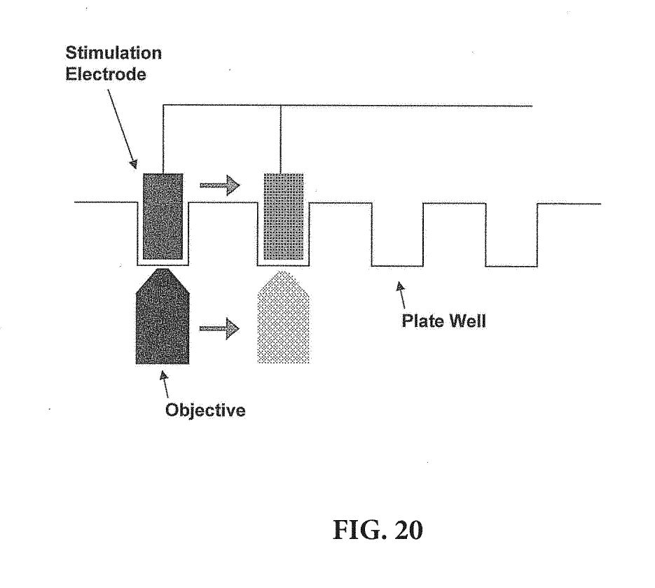

A high-content plate reader has been integrated with an electrical stimulation system that triggers action potentials in neurons in a single well while the synaptic vesicle cycle was being monitored in that well by the plate reader. The system was automated so that the synaptic vesicle cycle assay could be performed on an entire plate without human intervention. Moreover, plate-handling robots was used to automate the processing of multiple plates serial on a single instrument. An exemplary high-content screening system is illustrated in FIG. 20.

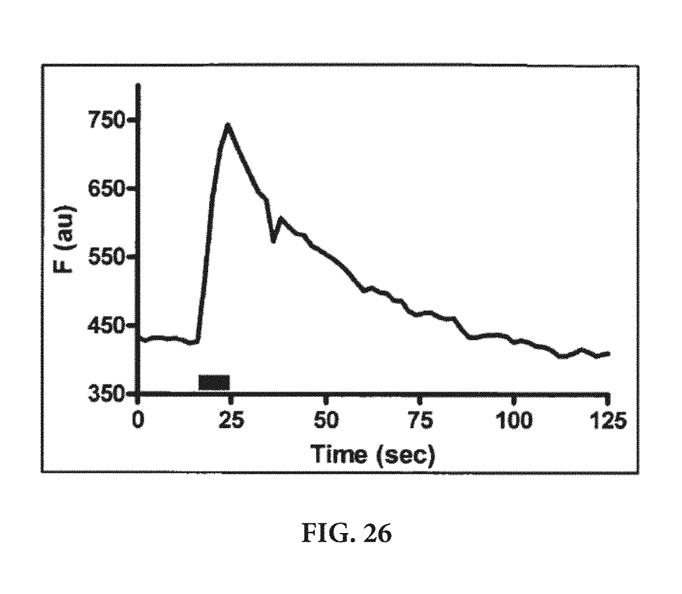

The synaptic vesicle cycle was measured using a high content imaging system. Results are shown in FIG. 26. Rat embryonic cortical neurons were plates into Greiner uClear 96-well plates and infected with the sypHy-AAV at 7 DIV. At 25 DIV, medium was replaced with assay buffer, and the plate was loaded onto a BD Pathway high-content imaging system. The Cellectricon CX3 electrode was placed into three wells. The neurons were imaged at 1 Hz on the Pathway and stimulated for 10 sec at 30 Hz with 1 msec pulses using the CX3 electrode. The image time series was analyzed for change in fluorescence during the reading period. The high content imaging system was capable of measuring the synaptic vesicle cycle stimulated by the Cellectricon electrode system.

### Example 9: Presynaptic HTS Platform System (PHTSP)

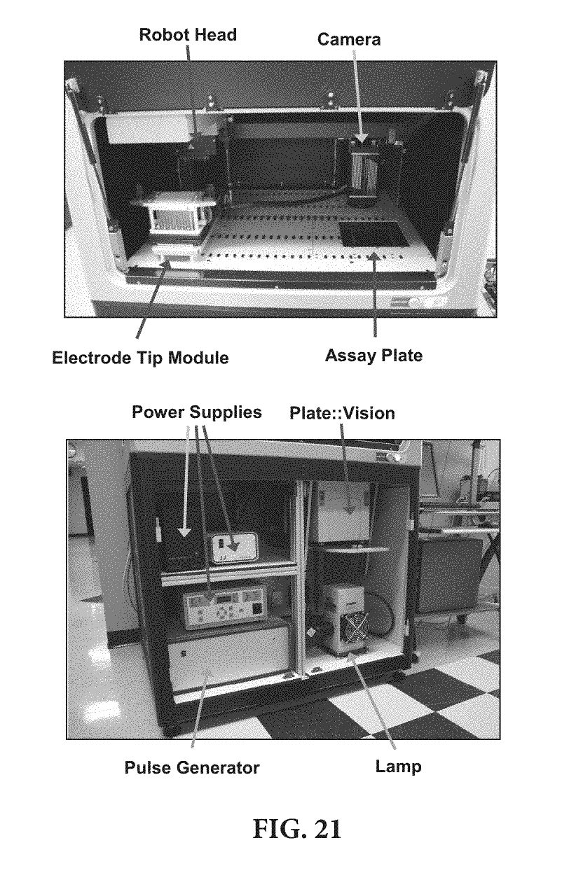

A presynaptic HTS platform system has been constructed (FIG. 21) and 96 parallel presynaptic assays were carried out in using the system.

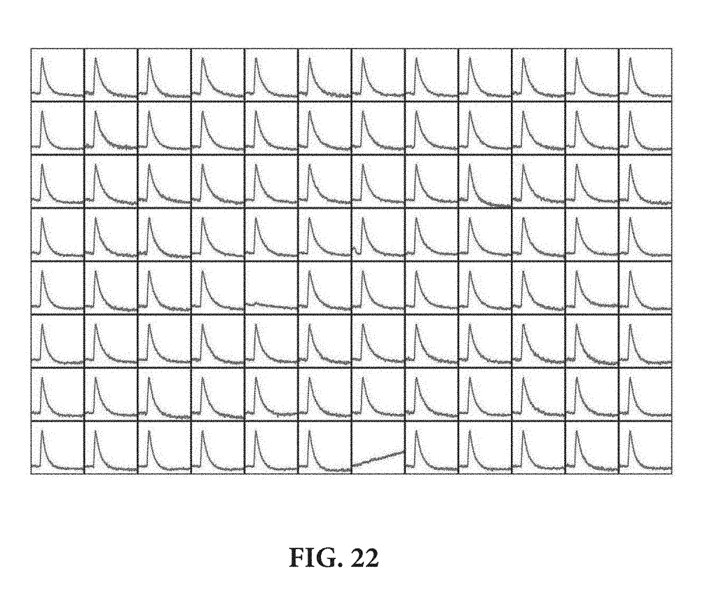

Rat embryonic forebrain neurons were plated into Greiner mClear 96-well plates and infected at 7 DIV with the hSyn-SypHy adeno-associated virus. At 25 DIV, cell culture media from a neuronal plate was replaced with pHluorin assay buffer, and the plate was loaded onto the platform. The plate was imaged at 1 Hz on the plate::vision plate reader and stimulated at 30 Hz for 10 sec with 15V, 185 ms pulses using the customized CellAxessHT electrode. The synaptic vesicle cycle was measured in all wells in parallel. FIG. 22 depicts the results from all wells, with the traces normalized to their peak response. The two failures are likely due to failure of the electrodes within those wells to make proper electrical contact with the assay buffer. These data show that the presynaptic HTS platform is capable of measuring the synaptic vesicle cycle in all wells of a 96-well plate in parallel.

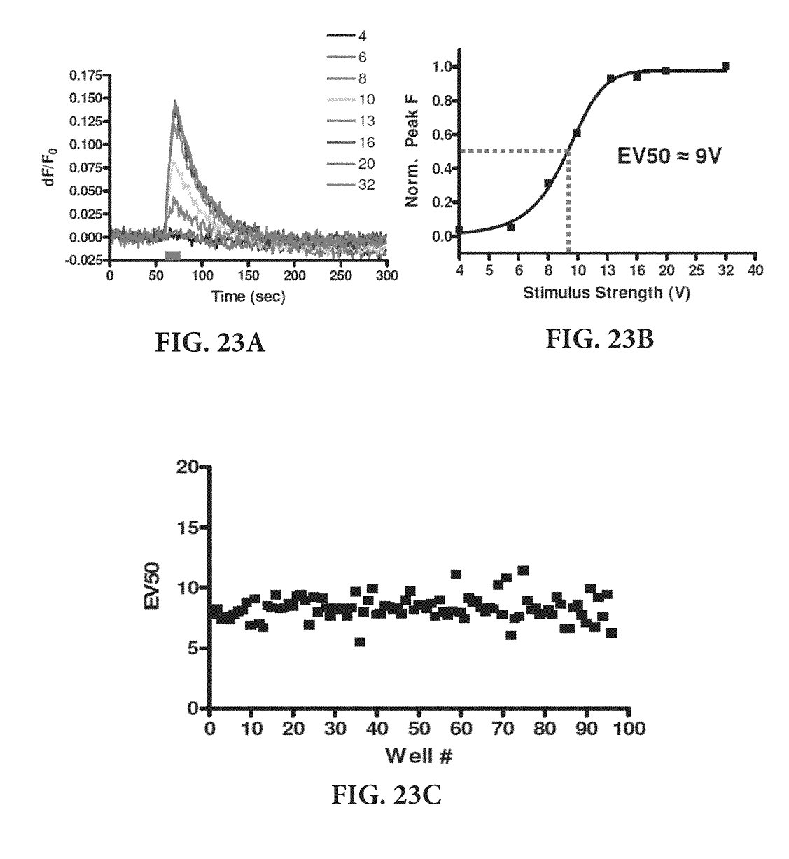

A uniformity analysis of the presynaptic HTS platform electrical stimulation system was performed. Rat embryonic forebrain neurons were plated in Greiner mClear 96-well plates and infected at 7 DIV with the hSyn-SypHy adeno-associated virus. At 25 DIV, cell culture media from the neuronal plate was replaced with pHluorin assay buffer, and the plate was loaded onto the platform. The plate was imaged at 1 Hz on the plate::vision plate reader and stimulated at 30 Hz for 10 sec with 185 ms pulses of increasing voltages from 0-32V using the customized CellAxessHT electrode. Results are shown in FIG. 23.

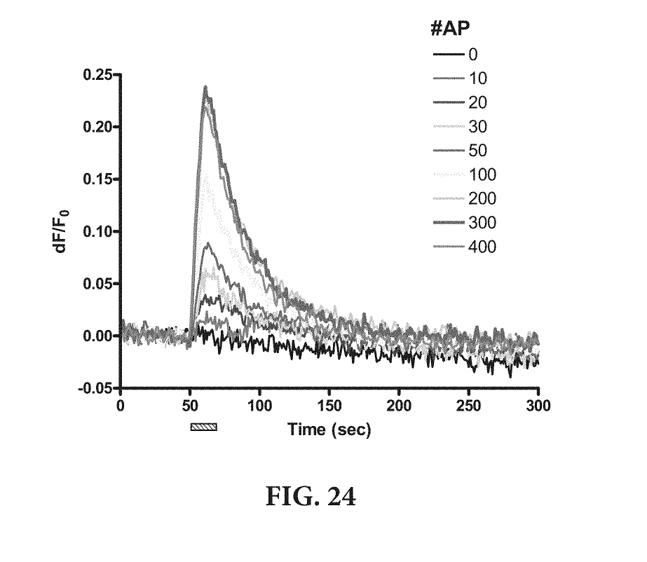

A sensitivity analysis of the presynaptic HTS platform was performed. Rat embryonic forebrain neurons were plated in Greiner mClear 96-well plates and infected at 7 DIV with the hSyn-SypHy adeno-associated virus. At 25 DIV, cell culture media from a neuronal plate was replaced with pHluorin assay buffer, and the plate was loaded onto the platform. The plate was imaged at 1 Hz on the plate::vision plate reader and stimulated at 10 sec with 15V, 185 ms pulses of increasing frequency from 1 to 40 Hz using the customized CellAxessHT electrode (slanted hatch filled bar). Under these conditions, the presynaptic HTS platform is capable of measuring the synaptic vesicle cycling response to as few as 20 action potentials delivered over 10 sec (2 Hz). (FIG. 24).

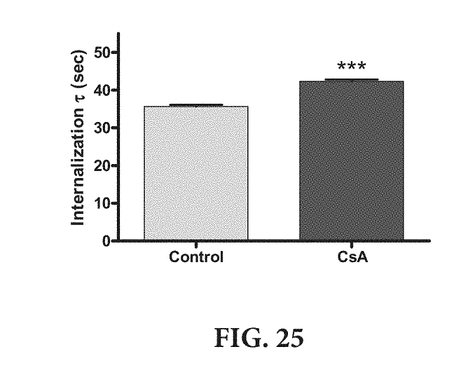

Compound-induced changes in the synaptic vesicle cycling with the presynaptic HTS platform were detected. Rat embryonic forebrain neurons were plated in Greiner mClear 96-well plates and infected at 7 DIV with the hSyn-SypHy adeno-associated virus. At 25 DIV, cell culture media from a neuronal plate was replaced with pHluorin assay buffer, and the calcineurin inhibitor cyclosporine A (CsA; 20 μM) was added to alternating columns. Edge wells were avoided in this experiment. The plate was loaded onto the platform, imaged at 1 Hz on the plate::vision plate reader and stimulated at 30 Hz for 10 sec with 15V, 185 ms pulses using the customized CellAxessHT electrode. Synaptic vesicle cycle waveforms were collected and the time constant (t) for reinternalization of the synaptic vesicles following the stimulation was calculated for each well. The reinternalization rate following calcineurin inhibition was slowed significantly (Control=36±0.5 sec, n=28; CsA=42±0.5 sec, n=29; p<0.0001). These data, depicted in FIG. 25, show that the platform is capable of detecting compound-induced alterations in the kinetics of the synaptic vesicle cycle.

### Example 10: Plate Reader Based Detection System

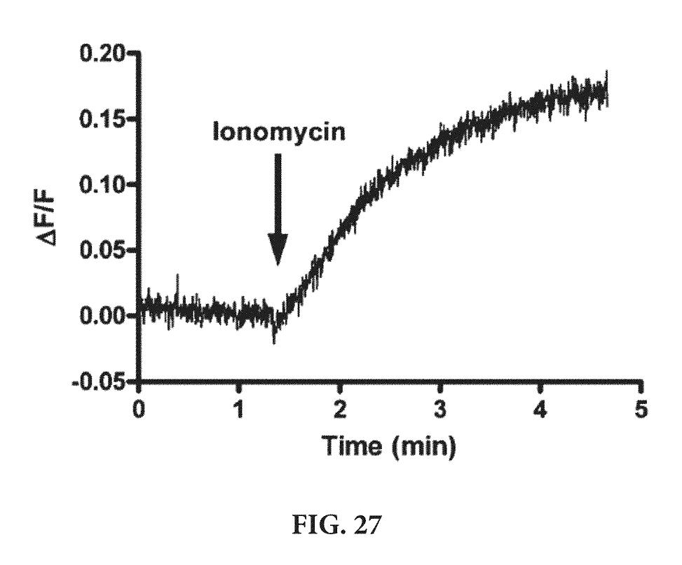

It was determined that a Fluoroskan Ascent FL plate reader is capable of measuring aspects of synaptic vesicle cycling, e.g., exocytosis, in neuronal cells expressing a synaptophysin-pHluorin reporter. Rat embryonic cortical neurons were cultured in Greiner uClear 96-well plates. At 7 DIV, cultures were infected with sypHy-AAV. At 33 DIV, culture media was replaced with assay buffer, the plate was loaded onto the Fluoroskan Ascent FL plate reader, and GFP-fluorescence was recorded at 5 Hz. This plate reader uses a photomultiplier tube (PMT) to measure the fluorescence levels in a single well at a time. A baseline fluorescence level was determined, ionomycin (10 uM) was added to induce synaptic vesicle exocytosis, and the resulting change in fluorescence was measured on by plate reader. Ionomycin resulted in ˜15% increase in fluorescence intensity, showing that the Fluoroskan Ascent FL, with its PMT optics, is capable of measuring the synaptic vesicle cycle using the sypHy reporter. (FIG. 27.)

### EQUIVALENTS AND SCOPE

Those skilled in the art will recognize, or be able to ascertain using no more than routine experimentation, many equivalents to the specific embodiments, described herein. The scope of the present invention is not intended to be limited to the above Description, but rather is as set forth in the appended claims.

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. It is also noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any cell type; any neuronal cell; any reporter of synaptic vesicle cycling; any electrical stimulation system; any imaging system; any synaptic vesicle cycling assay; any synaptic vesicle cycle modulator; any method of use; etc.) can be excluded from any one or more claims, for any reason, whether or not related to the existence of prior art.

We claim:

1.-226. (canceled)

227. A platform comprising:
a) a plurality of wells;
b) an optical stimulation system configured to initiate action potentials in excitable cells in the wells, wherein the optical stimulation system comprises an array of luminous devices that are spatially matched to the plurality of wells and adapted to illuminate the plurality of wells;
c) a detection system comprising at least one detector configured to detect a luminescent signal indicative of action potentials in the excitable cells.

228. The platform of claim 227, wherein the excitable cells are present in the plurality of wells.

229. The platform of claim 228, wherein the excitable cells are engineered to express light-gated ion channels.

230. The platform of claim 229, wherein the light-gated ion channel is a channel rhodopsin.

231. The platform of claim 229, wherein the optical stimulation system is configured to activate the light-gated ion channels to depolarize the excitable cells and initiate the action potentials in the excitable cells.

232. The platform of claim 229, wherein the optical stimulation system is configured to initiate the action potentials in the excitable cells by exposing the excitable cells to optical pulses.

233. The platform of claim 232, wherein the optical pulses are provided from one or more luminous systems.

234. The platform of claim 233, wherein the one or more luminous systems comprise light-emitting diodes, diode lasers, incandescent lamps, or neon lamps.

235. The platform of claim 234, wherein the one or more luminous systems emit a radiation at a wavelength to which the light-gated ion channels are responsive.

236. The platform of claim 227, wherein the optical system comprises lenses, optical filters, mirrors or other optical components that concentrate and/or direct emitted radiation onto the excitable cells.

237. The platform of claim 234, wherein the optical system comprises an array of luminous devices that are spatially matched to the plurality of wells comprising excitable cells, wherein the luminous devices are adapted to illuminate the plurality of wells comprising excitable cells.

238. The platform of claim 227, wherein the optical system comprises an array of luminous devices disposed in a linear array corresponding to a row or column of a multiwell plate comprising the plurality of wells.

239. The platform of claim 238, wherein optical pulses from the array of luminous devices initiate the firing of action potentials in the excitable cells.

240. The platform of claim 227, wherein the excitable cells comprise light-activated glutamate receptor (LiGluR) engineered to initiate action potential firing in the excitable cells.

241. The platform of claim 227, wherein the excitable cells comprise a modified kainate receptor with extracellular cysteine, to which a light-activated ligand can bind.

242. The platform of claim 241, wherein the optical system comprises one or more luminous devices configured to photoswitch the light-activated ligand.

243. The platform of claim 240, wherein the platform is arranged such that LiGluR or photoswitch activation induces high frequency firing of action potentials in the excitable cells.

244. The platform of claim 227, wherein the excitable cells comprise primary neuronal cells and/or differentiated from stem cells.

245. The platform of claim 227, wherein the excitable cells are neuronal cells, and wherein the luminescent signal is emitted from a reporter in the neuronal cells indicative of synaptic vesicle mobilization or neurotransmitter release.

246. The platform of claim 227, wherein the excitable cells are neuronal cells, and wherein the luminescent signal is emitted from a reporter of synaptic vesicle cycling in the neuronal cells.

247. The platform of claim 227, wherein the synaptic vesicle protein is a synapto-pHluorin.

248. The platform of claim 227, wherein the detection system comprises a plurality of detectors.
